(19) United States
(12) Patent Application Publication
(10) Pub. No.: US 2024/0352462 A1
(43) Pub. Date: Oct. 24, 2024

(54) ANTISENSE INHIBITORS OF MIR17HG PRE-RNA AS THERAPEUTIC AGENTS IN CANCER

(71) Applicant: Dana-Farber Cancer Institute, Inc., Boston, MA (US)

(72) Inventors: Nikhil C. Munshi, Needham, MA (US); Eugenio Morelli, Boston, MA (US); Sergei Gryaznov, Boston, MA (US)

(21) Appl. No.: 18/712,843

(22) PCT Filed: Nov. 29, 2022
(86) PCT No.: PCT/US2022/080563

§ 371 Date: May 23, 2024

Related U.S. Application Data
(60) Provisional application No. 63/284,527, filed on Nov. 30, 2021.

(51) Int. Cl. C12N 15/113; A61K 31/7125; A61P 35/00

(52) U.S. Cl. CPC C12N 15/1135; A61K 31/7125; A61P 35/00; C12N 2310/113; C12N 2310/315; C12N 2310/341; C12N 2310/3515; C12N 2310/3525

(57) ABSTRACT

Disclosed are antisense oligonucleotide inhibitors to MIR-17-92a-1 Cluster Host Gene (MIR 17HG) pre-RNA for treating or ameliorating diseases in which the MIR 17HG pre-RNA plays a role.

### RELATED APPLICATIONS

This application claims the benefit of priority under 35 U.S.C. § 119 (e) to U.S. Provisional Application No. 63/284,527, filed Nov. 30, 2021, which is incorporated herein by reference in its entirety.

### SEQUENCE LISTING

The instant application contains a Sequence Listing which has been submitted electronically in XML format and is hereby incorporated by reference in its entirety. Said XML copy, created on Nov. 28, 2022, is named 52095_753001WO_SL.xml and is 71 KB bytes in size.

### BACKGROUND OF THE DISCLOSURE

In the human genome, gene loci harboring long non-coding RNAs (lncRNAs) outnumber protein-coding genes and are susceptible to the same oncogenic pathogenetic events (Hon et al., Nature 543:199-204 (2017); Wang et al., Cancer Cell 33:706-720 (2018)). These RNA molecules are defined as having a length greater than 200 nucleotides (nt) and a lack of protein-coding potential, and therefore represent a diverse array of functional entities (Ulitsky et al., Cell 154:26-46 (2013)). LncRNAs are often classified in four separate subgroups based on their location relative to protein-coding genes: exonic, intronic, overlapping, intergenic, sense and antisense lncRNAs; alternatively, they can be functionally classified into cis- and trans-acting lncRNAs. Ulitsky et al., Cell 154:26-46 (2013). Trans-acting lncRNAs are of special interest for their diverse mechanisms of action, such as their role as precursor molecules for the biogenesis of mature microRNAs (miRNAs) (Lu et al., Nat. Med. 23:1331-1341 (2017)) or through their direct interactions with proteins and nucleic acids to regulate protein function and/or stability (Tseng et al., Nature 512:82-6 (2014)). Aberrant expression and function of lncRNAs have been implicated in the progressive gain of a malignant phenotype by tumor cells (Gutschner and Diederichs, RNA. Biol. 9:703-19 (2012)).

### SUMMARY OF THE DISCLOSURE

A first aspect of the present disclosure is directed to an antisense oligonucleotide (ASO) that binds the MIR-17-92a-1 Cluster Host Gene (MIR17HG) pre-RNA under physiological conditions, wherein the ASO is 15 to about 30 nucleotides in length and the MIR-17-92a-1 Cluster Host Gene (MIR17HG) pre-RNA has the nucleic acid sequence of SEQ ID NO: 1.

Another aspect of the present disclosure is pharmaceutical composition containing a therapeutically effective amount of the antisense oligonucleotide and a pharmaceutically acceptable carrier.

Yet another aspect of the present disclosure is a method of treating a disease in which aberrant expression and function of the MIR17HG pre-RNA plays a role. The method entailing administering to the subject the pharmaceutical composition.

Yet another aspect of the present disclosure is a kit containing a therapeutically effective amount of an ASO that binds MIR17HG pre-RNA, and printed instructions for using the first active agent in the treatment of a disease in which aberrant expression and function of MIR17HG pre-RNA plays a role in a subject.

Working examples described herein demonstrate ASOs that bind MIR17HG pre-RNA result in decreased multiple myeloma tumor growth in vivo without toxicity, in a microRNA and DROSHA independent manner.

### BRIEF DESCRIPTION OF THE DRAWINGS

FIGS. 1A-1D are a set of schematics and dot and line plots that illustrate the genome-wide CRISPRi viability screen that identified MIR17HG as a leading dependency in multiple melanoma (MM). FIG. 1A schematically illustrates the viability screen. FIG. 1B is a set of line plots showing the RRA-based ranked analysis of lncRNA dependencies in the secondary screen. FIG. 1C is a set of line plots showing a CCK-8 proliferation assay of MM cell lines. FIG. 1D is a set of line plots showing a CCK-8 proliferation assay of MM cell lines transfected with ASOs targeting the MIR17HG pre-RNA or a non-targeting ASO (NC).

FIGS. 2A-2G are a set of schematics, line, bar, and survival plots that show how the lncRNA Inc-17-92^{TV1}, also referred herein as the Regulator of Lipogenesis (RROL) mediates dependency in microRNA-independent manner. FIG. 2A schematically illustrates an overview of the MIR17HG locus, including both lncRNA (RROL) and miRNA (miR-17-92) derived transcripts. FIG. 2B is a set of line plots that show the prognostic significance (PFS and OS) of high RROL expression (top quartile) in 3 large cohorts of MM patients. FIG. 2C is a set of bar plots that illustrate a CCK-8 proliferation assay in AMO1 and H929 cells transduced with pri-mir-17-92 (pri-miR) or GFP control. FIG. 2D is a set of bar plots and images that illustrate a CCK-8 proliferation assay of DROSHA WT or KO AMO1 and H929 cells exposed to ASO1. FIG. 2E is a line plot that illustrates the effects of RROL depletion in a matrigel-based AMO1DR-KO xenograft in NOD SCID mice. FIG. 2F is a survival plot that illustrates a survival analysis of tumor injected mice. FIG. 2G is a set of bar plots that show a CCK-8 proliferation assay in HCT-116 and DLD-1 colorectal cancer cell lines expressing either a WT or mutated (−/−) Dicer.

FIGS. 3A-3G are a set of diagrams, images, bar, and dot plots that show RROL's interaction with chromatin to regulate gene expression. FIG. 3A is a diagram that shows Transcriptomic analysis after RROL depletion in MM cell lines, either DROSHA WT (AMO1, H929) or KO (AMO1^{DR-KO}) and shows commonly downregulated genes (adj p<0.05; lfc<−1). FIG. 3B is a bar plot that illustrates a qRT-PCR analysis of RROL targets in CD138+ cells from 3 MM patients exposed to ASO1 for 24 h. FIG. 3C is a dot plot and a set of correlation plots showing the correlation between RROL targets (mRNA) and RROL in CD138+MM patient cells together and alone for each RROL targets. FIG. 3D is a bar plot that illustrates a GLuc/SEAP dual reporter assay showing reduced activity of ACC1, ANO6, CCDC91, EPT1, EXT1, FER, and KIAA1109 promoter activity after RROL knockdown using ASO1. FIG. 3E is a set of line plots that illustrate a CCK-8 proliferation assay in 5 MM cells lines after transfection with siRNAs against RROL targets. FIG. 3F is a bar plot that illustrates a ChIRP-qPCR analysis showing effective amplification of ACC1 promoter in chromatin purified using 2 RROL antisense probe sets (ps1 and ps2), compared to chromatin purified using LacZ antisense probes (negative control). FIG. 3G is a set of images and a dot plot that illustrates a (left) snapshot obtained by dual RNA-FISH analysis of ACC1 pre-mRNA (green) and RROL (purple) in a representative AMO1 cell; (right) box plot showing the distance (nm) of ACC1 pre-RNA spots to the nearest RROL spots or to the nearest random spots.

FIGS. 4A-4G are a set of bar and images that illustrate RROL's promotion of MYC occupancy at the ACC1 promoter. FIG. 4A is bar plots that show upstream regulator analysis of transcriptional changes after 3 days of gymnotic exposure to ASO1 in H929 and AMO1 cells. FIG. 4B a set of bar plots and images that illustrate a ChIP-qPCR analysis of MYC occupancy at ACC1 promoter in AMO1, H929 and U266MYC-exposed for 24 h to ASO1 or NC (vehicle). FIG. 4C is a bar plot and image that illustrates a qRT-PCR analysis of ACC1 mRNA in P493-6 cells exposed for 2 days to either doxycycline or DMSO to knockdown MYC and for additional 2 days, exposed to either ASO1 or vehicle (NC) to deplete RROL. FIG. 4D is a set of images that illustrates a Co-IF/FISH analysis of ACC1 pre-RNA (red), MYC protein (green) and RROL (purple). FIG. 4E illustrates a qRT-PCR analysis of RROL (detecting isoform 2) in RIP material precipitated using an anti-MYC antibody (a-MYC) or IgG control. FIG. 4F illustrates a Western blot analysis of MYC in RPPD material precipitated with control RNA or RROL transcripts-1 and -2. FIG. 4G illustrates an RNA Y3H using MYC as hybrid protein 2 and, as hybrid RNAs, either a negative control RNA (−) or RROL transcripts-1 and -2.

FIGS. 5A-5I are a set of schematics, images, and bar plots that illustrate that RROL mediates the assembly of a MYC-WDR82 transcriptional complex, leading to transcriptional and epigenetic activation of ACC1. FIG. 5A schematically shows integrated BioID and Co-IP/MS assays to explore the MYC-protein interacting network in presence or absence of RROL depletion. FIG. 5B is an image that illustrates a western blot analysis of WDR82 in RPPD material precipitated with RROL-1 and RROL-2 or with control RNA. FIG. 5C is an image that illustrates an RNA Y3H using WDR82 as hybrid protein 2 and, as hybrid RNAs, either a negative control RNA (−) or RROL transcripts-1 and -2. FIG. 5D is a bar plot and image that illustrates a ChIP-qPCR analysis of H3K4me3 occupancy at ACC1 promoter in AMO1 after silencing of WDR82. FIG. 5E is a bar plot and image that illustrates a ChIP-qPCR analysis of MYC occupancy at ACC1 promoter in AMO1 after silencing of WDR82. FIG. 5F is a bar plot and image that illustrates a qRT-PCR analysis of ACC1 mRNA after transfection of siRNA targeting WDR82 (siWDR82-1 or -2) in AMO1. FIG. 5G is a bar plot and image that illustrates a ChIP-qPCR analysis of WDR82-GFP occupancy at ACC1 promoter in AMO1 exposed for 24 h to gymnotic ASO1. FIG. 5H is a bar plot and an image that illustrates a ChIP-qPCR analysis of H3K4me3 occupancy at ACC1 promoter in AMO1 and H929 exposed for 24 h to gymnotic ASO1. FIG. 5I is a set of images of western blots that shows H3K4me3 levels at the ACC1 promoter site.

FIGS. 6A-6E are a set of bar plots, flow cytometry plots, and schematics that show that the RROL/MYC-ACC1 axis regulates de novo lipogenesis. FIG. 6A illustrates the incorporation of ¹⁴C-glucose into lipids following either RROL, MYC, or ACC1 depletion/inhibition in MM cell lines or CD138+MM patient cells. FIG. 6B is a set of bar plots that show liquid chromatography-mass spectrometry (LC-MS) based lipid profiling after RROL inhibition in MM cells. FIG. 6C is a bar plot that shows the effect of palmitate on anti-proliferative effects of RROL depletion in MM cells. FIG. 6D is a set of flow cytometry plots that show the effect of palmitate on pro-proliferative effects of RROL depletion in MM cells. FIG. 6E schematically illustrates the RROL pathway as it affects MM cell growth.

FIGS. 7A-7J are a set of dot, bar, and survival plots and images that depict therapeutic inhibitors of RROL that exert potent anti-tumor activity in vitro and in vivo in animal models of human MM. FIG. 7A schematically illustrates the multi-step screen to develop RROL therapeutic ASOs. FIG. 7B is a set of bar plots that illustrates a CCK-8 proliferation assay in a panel of 11 MM cell lines. FIG. 7C is a dot plot that illustrates a subcutaneous in vivo tumor growth of AMO1 cells in NOD SCID mice. FIG. 7D-FIG. 7E are bar plots that illustrate a qRT-PCR analysis of RROL (FIG. 7D) and RROL targets (FIG. 7E) in AMO1 xenografts. FIG. 7F is a bar plot that illustrates a lipid profiling analysis showing modulation of tripalmitin in tumors. FIG. 7G is a dot plot and images that illustrate a BLI-based measurement of in vivo tumor growth of MOLP8-luc+ in NSG mice. FIG. 7H is a survival plot from experiment in FIG. 7E, black arrows indicate treatments. FIG. 7I is a line plot that shows human k light chain ELISA-based measurement of in vivo tumor growth of MM patient cells in NSG mice (PDX-NSG). FIG. 7J is a bar plot that shows qRT-PCR analysis of BCL2L11 in tumors retrieved from animals treated with G2-15b*-TO (G) or SB9-19-TO (SB) or vehicle (NC) as control.

FIG. 8A-8C are a set of bar plots that illustrate the screen data and MIR17HG knockdown with and without antisense oligonucleotides. FIG. 8A is a bar plot that shows an analysis of screening data, with the upset plot showing the identification of cell-type unique and shared lncRNA dependencies in MM cells. FIG. 8B is a bar plot that shows knockdown of MIR17HG obtained in AMO1 engineered to express a dCas9-KRAB fusion protein and anti-MIR17HG gRNAs under the regulation of a conditional promoter. FIG. 8C is a bar plot that illustrates a knockdown of MIR17HG obtained in AMO1 transfected with two gapmeRs.

FIGS. 9A-9L are a set of box and bar plots that illustrate RROL expression and correlation with the miR-17-92 microRNAs. FIG. 9A is a box plot that shows RROL expression in newly diagnosed (ND) vs relapsed (R) MM patients. FIG. 9B is a box plot that shows RROL expression in ND vs R MM patients from GSE66293. FIG. 9C is a Spearman's correlation that shows the interrelationship between RROL and miR-17-92 in CD138+ cells from 140 MM patients analyzed by RNA-seq and miRNA profiling. FIG. 9D is a set of bar plots that illustrate qRT-PCR analysis of miR-17-92 microRNAs in AMO1 and H929 cells. FIG. 9E is a set of bar plots that illustrate a qRT-PCR analysis of miR-17-92 microRNAs in AMO1 and H929 either WT or KO for DROSHA. FIG. 9F is a set of bar plots that illustrate the knockdown of MIR17HG using 3 different ASOs, or a scramble control (NC), in AMO1^{DR-KO} and H929^{DR-KO}. FIG. 9G is an RNA-seq coverage plot and junction analysis for RROL in 4 MM samples. FIG. 9H is a bar plot that shows qRT-PCR analysis of RROL transcript variants 1 and 2 in AMO1. Primers amplify the regions indicated in FIG. 9G. FIG. 9I is box plots that show RNA-seq analysis of RROL expression (TPM) in MMRF/COMMpass dataset (n=720) and 60 MM cell lines. FIG. 9J is a line plot that shows the effects of RROL depletion in a matrigel-based AMOIDR-KO xenograft in NOD SCID mice. FIG. 9K is a bar plot that shows a CCK-8 proliferation assay in HCC-116 cells transfected with ASOs targeting the 5′end (5′-ASO) of MIR17HG pre-RNA or a scrambled control (NC). FIG. 9L is bar plots that show Ectopic expression of pri-mir-17-92, lnc-17-92TV1, and lnc-17-92TV2, was confirmed by qRT-PCR showing: i) upregulation of miR-17 after ectopic expression of pri-mir-17-92, ii) upregulation of Inc-17-92^{TV1} after its ectopic expression, and iii) upregulation of Inc-17-92TV2 after its ectopic expression.

FIGS. 10A-10I are a set of images, heatmaps, and bar plots that illustrate RROL and miR-17-92 miRNA expression and localization in vitro. FIG. 10A is a bar plot that shows a sub-cellular qRT-PCR analysis of RROL in AMO1 and H929. FIG. 10B is a set of images that shows an RNA-FISH analysis of subcellular localization of RROL in AMO1. FIG. 10C is a heatmap that shows a qRT-PCR analysis of RROL and miR-17-92 miRNAs in AMO1 gymnotically exposed to ASO1 or ASO-1-NC. FIG. 10D is a bar plot that shows a qRT-PCR analysis of RROL transcriptional targets in Daudi and Raji cells gymnotically exposed to ASO1 for 24 h. FIG. 10E is a bar plot that shows a qRT-PCR analysis of murine rrol, acel and fer in 5TGMI murine MM cells transfected with 3 different ASOs targeting the murine mir 17hg nascent RNA. FIG. 10F-FIG. 10G are bar plots that show qRT-PCR analysis of miR-17-92 miRNAs (black bars) and RROL transcriptional targets (white bars) in AMO1 transfected with pooled miR-17-92 miRNA inhibitors (FIG. 10F) or mimics (FIG. 10G). FIG. 10H-FIG. 10I are bar plots that show a ChIRP-qPCR analysis showing the % (FIG. 10H) or CT values (FIG. 101) of positive reactions (amplification) of GAPDH exon2 in chromatin purified using 2 RROL antisense probe sets (ps1 and ps2) or using LacZ antisense probes (negative control).

FIGS. 11A-11G are a set of photos and a bar plot that show RROL promotes MYC occupancy at the ACC1 promoter. FIG. 11A is a western blot of MYC in U266 cells WT or infected with a vector carrying the expression of FLAG or MYC-FLAG. FIG. 11B is a schematic illustration of isoforms of RROL. FIG. 11C is a qRT-PCR of RROL isoforms −2 and −2 after immunoprecipitation with anti-MYC antibody (a-MYC) or IgG control. FIG. 11D is a bar plot that shows qRT-PCR analysis of ACC1 mRNA following treatment with MYC inhibitor 10058-F4 in MM cell lines AMO1 and H929. FIG. 11E is a bar plot that shows incorporation of C¹⁴-glucose into lipids following treatment with MYC inhibitor 10058-F4 in MM cell lines AMO1 and H929. FIG. 11F is a bar plot that shows a CCK-8 proliferation assay in U266^{MYC-} and U266^{MYC+} after transfection with siRNA targeting ACC1 or a scramble siRNA (NC). Cell viability was measured 48 h after transfection and it is represented as % of NC transfected cells. FIG. 11G is a photograph of a western blot analysis of MYC in RPPD material precipitated with control RNA or truncated versions of Inc-17-92TV1; 5% input is used as a reference.

FIGS. 12A-12D are a set of images that shows MYC and GFP expression after RROL knockdown or WDR82-GFP fusion protein expression. FIG. 12A is an image that illustrates western blot analysis of MYC in FBA-MYC cells with or without doxycycline to induce the FBA-MYC fusion protein and transfected with either NC or ASO1 to knockdown RROL. FIG. 12B is an image that illustrates western blot analysis of GFP in AMO1 cells WT or infected with a vector carrying the expression of GFP or WDR82-GFP. FIG. 12C is an image that shows western blot analysis of WDR82 in RPPD material precipitated with control RNA or truncated versions of Inc-17-92TV1. FIG. 12D is a set of images and a bar plot that shows western blot analysis of WDR82, H3, H3H3K4me1, H3H3K4me2, and H3H3K4me3 after silencing of WDR82 with a siRNA pool (n=4) in H929 (48 h time point). Lamin A/C was used as the protein loading controls (nuclear lysates), and ii. is the quantification of densitometry analysis.

FIGS. 13A-13C are a set of bar plots and photographs that show the effect of glucose update and expression of RROL targets after exposure to ASOs that bind RROL. FIG. 13A is a bar plot that shows the incorporation of C14-glucose into lipids after transfection of AMO1 with miR-17-92 anti-miRs. FIG. 1B is a bar plot that shows GEP analysis of miR-17-92's canonical targets BCL2L11 (BIM) and PTEN in AMO1 and H929 exposed to ASO1 for 36 h. FIG. 13C is a photograph of a western blot analysis of miR-17-92's canonical target BIM in AMO1 exposed to ASO1 for 36 h.

FIGS. 14A-14C are a set of bar plots that depict a multi-step screen to develop therapeutic ASOs targeting RROL. FIG. 14A is a bar plot that shows the results of step 1 identifying ASO-accessible stretches on RROL, 16 sequences (>20-mer) either in “G” or “SB” configuration. FIG. 14B is a bar plot that shows the results of step 2 optimizing the G2 and SB9 designs selected from step 1. FIG. 14C is a bar plot that shows the results of step 3 where step 2 molecules conjugated with palmitic acid (P), cholesterol (C), or tocopherol (T) were tested.

### DETAILED DESCRIPTION OF THE DISCLOSURE

### Definitions

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

The term “approximately” as used herein refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless otherwise clear from context, all numerical values provided herein are modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

### Antisense Oligonucleotides

In one aspect, the present disclosure provides antisense oligonucleotide (ASO) that binds the MiR-17-92a-1 Cluster Host Gene (MIR17HG) pre-RNA under physiological conditions.

The MIR17HG gene contains two lncRNAs, Inc-17-92^{TV1} and Inc-17-92TV2, and six miRNAs, miR-17, miR-18a, miR-19a, miR-19b, miR-20a, and miR-92a. The nucleic acid sequence of the Inc-17-92^{TV1} (also known as RNA Regulator of Lipogenesis (RROL), Inc-17-92TV1, and MIR17HGmiR-17-92) transcript is provided at NCBI Accession No. NR_027350, version NR_027350.1. Lnc-17-92^{TV1} is shown herein to provide a chromatin scaffold mediating a transcription factor complex to drive oncogenic gene translation. The nucleic acid sequence of the Inc-17-92TV2 transcript is provided at NCBI Accession No. NR_027349, version NR_027349.1.

The nucleic acid sequence of the MIR17HG pre-RNA is provided at Ensembl Accession No. ENSG00000215417, and set forth below (SEQ ID NO: 1):

| | |
|---|---|
| 1 | gaagctctcc tcgcggggcg ggccggccgg ccgcaccccc ggcctggggc ctccggtcgt |
| 61 | agtaaagcgc aggcgggcgg ggaggcggga gcaggagccc gcggccggcc agccgaagat |
| 121 | ggtggcggct actcctcctg gtgagtctgc ccgcccctcc ggcgacggag ggaaacctgt |
| 181 | tgtgtgcggc ccgggtctgg cgggcggggc ggagcggccc ggggcggact ggcccggggc |
| 241 | agcgtggcgg cggcggcgtg gccggggcgg gtctcggccg ttggccgccc cggcgtgtgg |
| 301 | cagccgcatc tggctgcccc ctcgctcgcc cgcgggccgg cggagggggg cagggccggg |
| 361 | gcgggagggt gggagggggc ggcgtgcgcg tggcggccgc gcccgggacc cgcgcagacc |
| 421 | ctgcctgggc cgacccgaag gcgggtgggc ggacggcgaa cacaatggcc cctcggggag |
| 481 | aggacgtgcg aggcccgtgc cttctccggg gcccggggcg cgcgcggggc gtggggtctc |
| 541 | tgggtaggaa agtttctccc gagggcgaga gttaaagcgc ctccagaaca aagcggcggc |
| 601 | ggcggcggca catggggcag gccgcgggcc gggagggggc gcgcccacga ggtacctgcg |
| 661 | cgccagcggg cggcgtggcg tgggcgggag cccgcggttc cccaaacttt gtacgcgcga |
| 721 | gggtgggcgg aggggcgccg agatcggcgc ggcctgggcg ccacccccgc tccgcgtggg |
| 781 | ctttgttagc ccgcgtgggc agcctcgggg cggggcccgc aacttccccg ccgtggccct |
| 841 | cggaggaggc cgcagtcggc ctcagccgcg gcgtggagcc gcctgcgccc ggccgcttgc |
| 901 | tgggagtgtg gcgcgggagg gccagcccgg ctcggcggga gcggcgtccc cgccgccatg |
| 961 | ttcctgcggg gcgggctgca cgggggtgag ggcgggggac atggcggcga ctgcgcgccg |
| 1021 | ccgccgattg ttcccggctt aggcctcggg ccgcgtgcga cgggcaccgc ggccgcggag |
| 1081 | cccccgcccc tctgggccgg gctcgggggg gctgggggac acaaaggagg ggcggcgcgc |
| 1141 | ccgcgtcccc gccgcactcg ggcctcggcg ccgccggtcg ccgcgcggct gccgccggga |
| 1201 | aacgggttgg gggggttgcc gcgtccggcg gggcctgact ctgacccgcc gccccctggc |
| 1261 | ggctacgcgg agaatcgcag ggccgcgctc ccccttgtgc gacatgtgct gccggcccgg |
| 1321 | gctccatgag cgtggcgggc actttgcagt ctcgggtgtt cctgcccggt cttctgttcc |
| 1381 | taaactgcag caaagggaaa aggaactgaa aaaggcaggc tcgtcgttgc aatatcacca |
| 1441 | aaagagaaaa ttaacggcat gccatcagga ccacagcagt tggagaaaca actctttatc |
| 1501 | ccggcttgca gccacgaggt cttgattggg ggaggggtgg tgaagaatag tctgtgggct |
| 1561 | gctttttttt tttcctttta ctggagctgt acagtggagt cggtgattgc tgctgatcat |
| 1621 | aatcaagtat tttaggagct tatttagaca tgtatctgat agctaaggat ttttcaactt |
| 1681 | tattctctta cgtatttttc aactgtaaat tattgggctt ttaaatcctg ctagtattgc |
| 1741 | tcgactctta ctctcacaaa tggatggaat taattgctgt taggaggttg gaaaatagca |
| 1801 | aatatagatt tggacggtgg tagtaatttt gagcaaataa tgttttatct tttttttcct |
| 1861 | tatttttccc tattccagtc atacacgtgg acctaactgc accagtagct tttctgagaa |
| 1921 | tacttgctga aaaggaagtt ttctggaatg ggtaagtgta ttctgatttt cttgaacttt |
| 1981 | tcttaaaaac aaatttttct tgctattaaa gttgaataaa taggattggt ttcttagaga |
| 2041 | gtaaaagtag gtgtttcttt ctttagacaa tgtacctttt ctgaaaaact aactcattaa |
| 2101 | gtacggattt gctaatttta aggtagtaaa attacagtgt aaatattcct gtacattttt |
| 2161 | ggaaactggc ttatgcagtt tacgaaatat aattttagac cctcttttaa gttgggtgat |
| 2221 | aaagtagata taacctgaga tgatagattt aaacaggata tttacgttct gctacaattg |
| 2281 | actgataaca cttgaagtgt agtctgaaca gtaattttgt taatcatttc aacaagtatt |
| 2341 | tgctaagtgg aagccagaag aggaggaaaa tgttttgcca cgtggatgtg aagatttcct |
| 2401 | ctaaaaggta cacatggact aaattgcctt taaatgttcc aaaattagtt ctcatttatt |
| 2461 | tgcagtctca ttttgttttg ttttttttct ctatgtgtca atccatttgg gagaggccag |
| 2521 | ccattggaag agccaccact tccagtgcta gttggatggt tggttatgat tgccttctgt |
| 2581 | aaagaattct taaggcataa atacgtgtct aaatggacct catatctttg agataattaa |
| 2641 | actaattttt tcttccccat tagggattat gctgaatttg tatggtttat agttgttaga |
| 2701 | gtttgaggtg ttaattctaa ttatctattt caaatttagc aggaaaaaag agaacatcac |
| 2761 | cttgtaaaac tgaagattgt gaccagtcag aataatgtca aagtgcttac agtgcaggta |
| 2821 | gtgatatgtg catctactgc agtgaaggca cttgtagcat tatggtgaca gctgcctcgg |
| 2881 | gaagccaagt tgggctttaa agtgcagggc ctgctgatgt tgagtgcttt ttgttctaag |
| 2941 | gtgcatctag tgcagatagt gaagtagatt agcatctact gccctaagtg ctccttctgg |
| 3001 | cataagaagt tatgtattca tccaataatt caagccaagc aagtatatag gtgttttaat |
| 3061 | agtttttgtt tgcagtcctc tgttagtttt gcatagttgc actacaagaa gaatgtagtt |
| 3121 | gtgcaaatct atgcaaaact gatggtggcc tgctatttcc ttcaaatgaa tgatttttac |
| 3181 | taattttgtg tacttttatt gtgtcgatgt agaatctgcc tggtctatct gatgtgacag |
| 3241 | cttctgtagc actaaagtgc ttatagtgca ggtagtgttt agttatctac tgcattatga |
| 3301 | gcacttaaag tactgctagc tgtagaactc cagcttcggc ctgtcgccca atcaaactgt |
| 3361 | cctgttactg aacactgttc tatggttagt tttgcaggtt tgcatccagc tgtgtgatat |
| 3421 | tctgctgtgc aaatccatgc aaaactgact gtggtagtga aaagtctgta gaaaagtaag |
| 3481 | ggaaactcaa acccctttct acacaggttg ggatcggttg caatgctgtg tttctgtatg |
| 3541 | gtattgcact tgtcccggcc tgttgagttt ggtggggatt gtgaccagaa gattttgaaa |
| 3601 | attaaatatt actgaagatt tcgacttcca ctgttaaatg tacaagatac atgaaatatt |
| 3661 | aaagaaaatg tgtaactttt tgtgtaaata catcttgtct tgttttcatt caaaaacatt |
| 3721 | tcacttttgg ggttgcgtgt cagatttggc agtataaatt ctggctatat tttttgttgt |
| 3781 | tagatttatt tggctgttaa gtattgcgat atgactaaac atactgtata cctgatgatc |
| 3841 | atctgtaaag ttagagtata tctttttgct ttctttggag ttagtgttat tccaggatat |
| 3901 | tttacttaat ctaaaagtta atttatgttg ctcatatatt actcaagtat ttaaatttag |
| 3961 | agagaatgcc gctctgttta aagcaatgtg taaagatgag tttttttaag catggaattt |
| 4021 | agggttgggg tacaatttgt ttctattaag caagtaccag tttaccaata catgagtaac |
| 4081 | tgaagtgtaa ctgttaaatg cttgtatact agtttttctt tctgattgtc agtgatttat |
| 4141 | aagctataaa tgaccaaggt cctcagactg cttttagcat ctgcaactta aaaaaatggg |
| 4201 | agttagaaaa agaacaaatg ctaaatagag taacagttaa atgtatgtgt acactcttcc |
| 4261 | caaatgccaa gagtgcagcg gtggggtgag attcagatat tcatttattt ctaagtctgt |
| 4321 | agttaacatt tatgttccct actccctacg taagccagac tttggcaaca gtgatagttg |
| 4381 | attccaggct tatttgactt aaagtcactg aagtggaaac taagaagtgg cagttagtgt |
| 4441 | tttacccagc atttctgcct tctctctttt cttcatgtgt ttttgtctct agcctatgtg |
| 4501 | tatttgtgta gaataatgtg ggatacctga ataatagatt taaaaggacc aagtggtaaa |
| 4561 | attgggccca agctgaagta caggcaaact tgatgtttga aagataagtt ttgagaaatg |
| 4621 | tcattgtatt ttggagtaaa agaggctatc ttagtaataa gaaataaact tccataacac |
| 4681 | taggttagac cacccaataa atctagaaat cagcttttaa aaatattgtc tgaagtctaa |
| 4741 | caaaagtttt cacctctaat gtgttcttta agaaatttaa ggaacttagc cttggattcc |
| 4801 | tgaatagaaa ggtaagaatt ctatcattct ggagttgatg aaaacataaa ttttcaggat |
| 4861 | gtgaaatgaa cagtgattta taaaatggaa atcaaattgt acattagcag agttcttaag |
| 4921 | ctttttgaat tgaaggagac ctaataattg tgtctttttg gttatttagt gacaaacgtg |
| 4981 | gctttcaaac tatgcttaaa aagttccggc tggacacggt ggctcacacc tataatccta |
| 5041 | gcacttgggg aggctgaggc agacggatta cctgaggtca ggagttcgag accaacctgg |
| 5101 | ccgacatggt gaaacgctgt ctctactaaa aatataaaaa attagccggg tgcagtggcg |
| 5161 | tgcacctgta atcccagcta ctctggaggc tgaggcagga gaatcacctg aacctgggag |
| 5221 | gtggaggttt cagtgagctg agatcctgcc actgcactcc agcctgggcg caagaccaag |
| 5281 | acttaaacgc aaaaaaaaaa aaaaaaaaaa aaaaaaagtt tcataataca gcatggtctg |
| 5341 | gtagtttgca aaatggtgtg cttttgggga gatacactag caattttttt aaaaactgga |
| 5401 | acagtgtgat aggaagcctg ctggatgatt tcttaaatat tctaaaatgt aagtcaaata |
| 5461 | tgttttaata acaaagactt aaatggcttt tctccctaga gactgaaact agtattcatt |
| 5521 | gtgttcagaa cttaattggg cttgaactga gatttaaatc taataaacaa gttaataaat |
| 5581 | gtgtatgttt tgttgtgggt ttggtagtga tctgtggttc tatagggttt aataggaatt |
| 5641 | gcttttgatt tgtttctggc tttagaatgt gaggcaaatt ttacattctt ggttctatta |
| 5701 | agattttctt aggcatgcta acatgccaac aaaaagccat gtaagtattg tataaaaaga |
| 5761 | ttcacattgt taatttagcc attttgaaat tcagatgagt gagcaagttg ataatggcct |
| 5821 | catctctgac ctgagaaaaa acaactttga cccttgttct taaaatgctt taaccttgaa |
| 5881 | gttgcttgag acttaagagg tcatgttgct ttaggtttaa taaatagcct taactatttg |
| 5941 | gaggggaaaa aatgggtcaa cttttttttt tttttttggc gtttgcatgt acaactttct |
| 6001 | atttttagcc tatatttgga aagaaagcac ttaacatttt aggaattctt tttaaagctg |
| 6061 | cttgcaaagt gttggtgatt ttactgaaaa cttttgagat cttcatttta caggcagacc |
| 6121 | tgtctaacta caagccagac ttgggttttc tcctgtagtt tgaagacaca ctgactcctg |
| 6181 | acaaaatgca gcctgcaact tcctggagaa caactcagtg tcacattaaa gtttattatg |
| 6241 | tatttaatga tacactgttt aattgacagt tttgcatagt ttgtctaact ttagagaatt |
| 6301 | aagagcctct caactgagca gtaaaggtaa ggagagctca atctgcacag agccagtttt |
| 6361 | tagtgtttga tggaaataag atcatcatgc ccacttgaga cttcagatta ttctttagct |
| 6421 | tagtggttgt atgagttaca tcttattaaa gtcgaaatta atgtagtttt ctgccttgat |
| 6481 | aacatttcat atgtggtatt agttttaaag ggtcattagg aaaatgcaca tattccatga |
| 6541 | attttaagac ccatagaaaa gttgaagaat gcttaatttt cttatccagt aatgtaaaca |
| 6601 | cagagacaga acattgagat gtgcctagtt ctgtatttac agtttggtct ggctgtttga |
| 6661 | gttctagcgc atttaatgtt aataaataaa atactgcatt ttaaagctgt taagaaattg |
| 6721 | tccagaacga gaatattgaa ataaaaactt caaggttatt |

The term “antisense oligonucleotide” (abbreviated ASO) as used herein refers to a non-naturally occurring polymer of nucleotides (oligomer) capable of binding a target RNA molecule.

The term “nucleotide,” unless specifically sated or obvious from context, includes nucleosides that have a ribose sugar (i.e., a ribonucleotide that forms ribonucleic acid, RNA) or a 2′-deoxyribose sugar (i.e., a deoxyribonucleotide that forms deoxyribonucleic acid, DNA) and a phosphate. Nucleotides serve as the monomeric units of nucleic acid polymers or polynucleotides. The four nucleobases in DNA are guanine (G), adenine (A), cytosine (C) and thymine (T). The four nucleobases in RNA are guanine (G), adenine (A), cytosine (C) and uracil (U). In the present context, it is understood that the ASOs are exogenous to the cells into which they may be introduced.

ASOs may be made up of ribonucleotides, deoxyribonucleotides, modified ribonucleotides, modified deoxyribonucleotides, or a combination thereof. ASOs modulate target RNAs by hybridization through at least partial complementary region(s). ASO modulation mechanisms include transcriptional arrest, RNA synthesis disruption (e.g., at various stages including capping, splicing, and/or transport from nucleus to cytoplasm), ribosome attachment, ribonuclease (RNAse) H recruitment, degradation of mRNA (e.g., gapmer ASOs), translational arrest (e.g., ASO binding to a target RNA blocking translation), or steric blocking of target RNA by ASO hybridization.

The terms “gapmer” and “gapmeR” are used herein to refer to short DNA ASO structures with RNA-like wing segments on both sides of a gap, or main region. These linear DNA molecules are designed to hybridize to a target to degrade an RNA through the induction of RNase H cleavage. Binding of a gapmer to the target RNA has a higher affinity due to the modified RNA-like flanking regions, as well as resistance to ASO degradation by nucleases. The gapmer gap contains at least one modification that is different from that of modifications in one or both wings. Such modifications include nucleobase, monomeric linkage, and sugar modifications.

In some embodiments, the ASO binds to the 5′ terminal region of the MIR17HG pre-RNA. The term “terminal region” when used in the context of the MIR17HG pre-RNA, refers to the first 20% of the 5′ terminal nucleotides. In some embodiments, the ASO binds within the first 10%, the first 15%, or the first 20% of the 5′ terminal nucleotides of MIR 17HG pre-RNA. In some embodiments, the ASO binds within the 3′ terminal region of the MIR 17HG pre-RNA.

In some embodiments, the ASO targets an intronic region proximate to the 5′ terminal end of the MIR 17HG pre-RNA. The term “intronic region” as used herein refers to regions of an RNA molecule that are removed from a pre-RNA in the course of transcription. In some embodiments, the ASO targets a region of MIR17HG pre-RNA from which the miRNA primary transcript (pri-miRNA) is spliced (e.g., the pri-mir-17-92). A pre-miRNA is a longer sequence from which a mature miRNA is derived.

The ASOs disclosed herein bind to the MIR17HG pre-RNA under physiological conditions. The term “physiological conditions” as used herein refers to conditions that are normally encountered in mammalian in vivo conditions, for example, an isotonic solution (about 0.9% normal saline) at body temperature (about 37° C.), at physiological pH (within the range of about 7.3 to about 7.5).

The minimum percent complementarity of the ASO that is effective may be determined in accordance with standard procedures. In general, the ASO has about 85% to about 100% complementarity to the MIR17HG pre-RNA. In some embodiments, the ASO has 100% complementarity with the MIR17HG pre-RNA sequence that it targets. In some embodiments, the ASO is at least 99% complementarity, at least 98% complementarity, at least 97% complementarity, at least 96% complementarity, at least 95% complementarity, at last 90% complementarity, at least 85% complementarity to MIR17HG pre-RNA.

The ASOs have a length ranging from 15 to 30 nucleotides. In some embodiments, ASO is 30 or less nucleotides in length. In some embodiments, the ASO is 25 or less nucleotides in length. In some embodiments, the ASO is 24 or less nucleotides in length. In some embodiments, the ASO is 23 or less nucleotides in length. In some embodiments, the ASO is 20 or less nucleotides in length. In some embodiments, the ASO is 18 or less nucleotides in length. In some embodiments, the ASO is from 15 nucleotides to 24 nucleotides in length. In some embodiments, the ASO is from 18 nucleotides to 23 nucleotides in length. In some embodiments, the ASO is 18 nucleotides in length (which is referred to herein as an 18-mer). In some embodiments, the ASO is a 20-mer. In some embodiments, the ASO is a 21-mer. In some embodiments, the ASO is a 22-mer. In some embodiments, the ASO is a 23-mer. The term “mer” when used herein in reference to a ASO refers to a length of nucleotides. For example, the terms “16-mer” and “16mer” refers to a stretch of 16 nucleotides.

In some embodiments, the ASO is a single stranded RNA, single guide RNA (sgRNA) for the use with CRISPR cas9 gene editing systems. Nucleic acid sequences of representative anti-MIR17HG pre-RNA sgRNAs are set forth below:

MIR17HG pre-RNA sgRNA #1:
(SEQ ID NO: 2)
AGTGGCGCGAAGGCGCAGGT;
MIR17HG pre-RNA sgRNA #2:
(SEQ ID NO: 3)
GTGGCGCGAAGGCGCAGGTC;
MIR17HG pre-RNA sgRNA #3:
(SEQ ID NO: 4)
CCTCGCCCGAGGGCGCGAAG;
and
MIR17HG pre-RNA sgRNA #4:
(SEQ ID NO: 5)
GAGGGCGCGAAGTGGCGCGA.

In some embodiments, the ASO is single stranded DNA (ssDNA) molecule. Nucleic acid sequences of representative ssDNA anti-MIR17HG pre-RNA ASOs are set forth in Table 1.

TABLE 1
Nucleic acid sequences of representative
anti-MIR17HG pre-RNA ASOs

| Name | Sequence |
|---|---|
| ASO1 | TACTTGCTTGGCTT |
| (SEQ ID NO: 6) | |
| ASO2 | CACCGTCCAAATCTAT |
| (SEQ ID NO: 7) | |
| ASO3 | AGCACTCAACATCAGC |
| (SEQ ID NO: 8) | |
| 5′-ASO1 | CACCGTCCAAATCTAT |
| (SEQ ID NO: 9) | |
| 5′-ASO2 | GTATGACTGGAATAGG |
| (SEQ ID NO: 10) | |
| Murine aso1 | TACAGTGGAAATCGGC |
| (SEQ ID NO: 11) | |
| Murine aso2 | GCGAGCAAACACGAAA |
| (SEQ ID NO: 12) | |
| Murine aso3 | ACTTGGATTGGATGAG |
| (SEQ ID NO: 13) | |
| ASO-Non Targeting | GCTCCCTTCAATCCAA |
| Control (ASO-NC) | |
| (SEQ ID NO: 14) | |

ASOs may be further modified, with the addition of a chemical moiety or chemical modification to its nucleobases, nucleotides, or internucleoside linkages. ASO modifications enhance stability in vivo, improve specificity, and reduce toxic side effects. A representative modification includes modification of nucleotides with 2′-O-methoxyethylribose (MOE) groups.

The MIR17HG pre-RNA-binding ASOs may be prepared in a G configuration, in which the ASO contains 5′ terminal nucleotides modified with 2′-MOE, unmodified DNA (a “DNA gap”), and 3′ terminal nucleotides modified with 2′-MOE. Alternatively, MIR17HG pre-RNA-binding ASOs may be prepared in a SB configuration, in which all of the nucleotides in the ASO are modified with 2′MOE. Nucleic acid sequences of representative ASOs are set for in Table 2 Error! Reference source not found.

Gapmer ASOs contain modified nucleotides terminal to a DNA gap. In some embodiments the DNA gap is 5, 6, 7, 8, 9, 10, 11, 12, or 15 nucleotides in length. In some embodiments, the ASO is a 5-8-5 gapmer, where a DNA gap is flanked 5′ by a 5mer of 2′-MOE chemically modified nucleotides, as well as flanked 3′ by a 5mer of 2′-MOE chemically modified nucleotides.

TABLE 2
Nucleic acid sequences of representative
MIR17HG pre-RNA-binding ASOs.

| Name | Sequence |
|---|---|
| ASO 1 | CCCGGCCGCCTCCCCCCGTCCC |
| (SEQ ID NO: 15) | |
| ASO 2, selected | CCCTCCCACCCTCCCCCGCC |
| as G molecule | |
| (G2) (20mer) | |
| (SEQ ID NO: 16) | |
| ASO 3 | CCCCCCCGACCCCCTGTGTTT |
| (SEQ ID NO: 17) | |
| ASO 4 | TCCTCCAACCTTTTATCGTTT |
| (SEQ ID NO: 18) | |
| ASO 5 | TCGTCCTTTTTTCTCTTGT |
| (SEQ ID NO: 19) | |
| ASO 6 | CTCCCCTTTTTTACCCAGTT |
| (SEQ ID NO: 20) | |
| ASO 7 | CCCTCTCCGGTCGGTAACCTT |
| (SEQ ID NO: 21) | |
| ASO 8 | TCGTCCTTTTTTCTCTTGTAGT |
| (SEQ ID NO: 22) | |
| ASO 9, selected | CCACCCCTAACACTGGTCTTCT |
| as SB molecule | |
| (SB9) (22mer) | |
| (SEQ ID NO: 23) | |
| ASO 10 | CCCCTCCGCCCTCGTCCTC |
| (SEQ ID NO: 24) | |
| ASO 11 | TCTCTCATTTTCATCCAC |
| (SEQ ID NO: 25) | |
| ASO 12 | AGAGTAAAACAAAACAAAA |
| (SEQ ID NO: 26) | |
| ASO 13 | ACAAAATAGAAAAAAAAGGA |
| (SEQ ID NO: 27) | |
| ASO 14 | ATAAAAAGGGATAAGGTCAG |
| (SEQ ID NO: 28) | |
| ASO 15 | TCGTCCTTTTTTCTCTTGTA |
| (SEQ ID NO: 29) | |
| ASO 16 | CCCTATGGACTTATTATCT |
| (SEQ ID NO: 30) | |

The representative ASOs in Table 2 may be modified, for example, the ASO 2 may be selected as the G configuration ASO for further modification, and the ASO 9 may be selected as the SB configuration ASO for further modification. These ASOs may be further modified, as illustrated in Table 3. In one embodiment, G2 fine tune 15 may be selected as the G configuration ASO (G2-15) as set forth in Table 3, and SB fine tune 19 may be selected as the SB configuration ASO (SB2-19), as set forth in Table 4 Error! Reference source not found.

TABLE 3
Nucleic acid sequences of representative ASOs
modifications based from the G2 molecule

| Name | Sequence |
|---|---|
| 20mers | |
| G2 fine tune 1 | gCCCTCCCACCCTCCCCCGC |
| (SEQ ID NO: 31) | |
| G2 fine tune 2 | cgCCCTCCCACCCTCCCCCG |
| (SEQ ID NO: 32) | |
| G2 fine tune 3 | ccgCCCTCCCACCCTCCCCC |
| (SEQ ID NO: 33) | |
| G2 fine tune 4 | cccgCCCTCCCACCCTCCCC |
| (SEQ ID NO: 34) | |
| G2 fine tune 5 | CCTCCCACCCTCCCCCGCCg |
| (SEQ ID NO: 35) | |
| G2 fine tune 6 | CTCCCACCCTCCCCCGCCgc |
| (SEQ ID NO: 36) | |
| G2 fine tune 7 | TCCCACCCTCCCCCGCCgca |
| (SEQ ID NO: 37) | |
| G2 fine tune 8 | CCCACCCTCCCCCGCCgcac |
| (SEQ ID NO: 38) | |
| 18mers | |
| G2 fine tune 9 | CCCTCCCACCCTCCCCCG |
| (SEQ ID NO: 39) | |
| G2 fine tune 10 | CCTCCCACCCTCCCCCGC |
| (SEQ ID NO: 40) | |
| G2 fine tune 11 | CTCCCACCCTCCCCCGCC |
| (SEQ ID NO: 41) | |
| G2 fine tune 12 | gCCCTCCCACCCTCCCCC |
| (SEQ ID NO: 42) | |
| G2 fine tune 13 | cgCCCTCCCACCCTCCCC |
| (SEQ ID NO: 43) | |
| G2 fine tune 14 | TCCCACCCTCCCCCGCCg |
| (SEQ ID NO: 44) | |
| G2 fine tune 15, | CCCACCCTCCCCCGCCgc |
| selected a G | |
| molecule (G2-15) | |
| (SEQ ID NO: 45) | |

TABLE 4
Nucleic acid sequences of representative ASOs
modifications based from the SB9 molecule

| Name | Sequence |
|---|---|
| 22mers | |
| SB9 fine tune 1 | aCCACCCCTAACACTGGTCTTC |
| (SEQ ID NO: 46) | |
| SB9 fine tune 2 | aaCCACCCCTAACACTGGTCTT |
| (SEQ ID NO: 47) | |
| SB9 fine tune 3 | aaaCCACCCCTAACACTGGTCT |
| (SEQ ID NO: 48) | |
| SB9 fine tune 4 | caaaCCACCCCTAACACTGGTC |
| (SEQ ID NO: 49) | |
| SB9 fine tune 5 | CACCCCTAACACTGGTCTTCa |
| (SEQ ID NO: 50) | |
| SB9 fine tune 6 | ACCCCTAACACTGGTCTTCaa |
| (SEQ ID NO: 51) | |
| SB9 fine tune 7 | CCCTAACACTGGTCTTCaaa |
| (SEQ ID NO: 52) | |
| SB9 fine tune 8 | CCTAACACTGGTCTTCaaaa |
| (SEQ ID NO: 53) | |
| 20mers | |
| SB9 fine tune 9 | CCACCCCTAACACTGGTCTT |
| (SEQ ID NO: 54) | |
| SB9 fine tune 10 | CACCCCTAACACTGGTCTTC |
| (SEQ ID NO: 55) | |
| SB9 fine tune 11 | ACCCCTAACACTGGTCTTCT |
| (SEQ ID NO: 56) | |
| SB9 fine tune 12 | aCCACCCCTAACACTGGTCT |
| (SEQ ID NO: 57) | |
| SB9 fine tune 13 | aaCCACCCCTAACACTGGTC |
| (SEQ ID NO: 58) | |
| SB9 fine tune 14 | CCCCTAACACTGGTCTTCa |
| (SEQ ID NO: 59) | |
| SB9 fine tune 15 | CCCTAACACTGGTCTTCaa |
| (SEQ ID NO: 60) | |
| 18mer | |
| SB9 fine tune 16 | ACCCCTAACACTGGTCTT |
| (SEQ ID NO: 61) | |
| SB9 fine tune 17 | CACCCCTAACACTGGTCT |
| (SEQ ID NO: 62) | |
| SB9 fine tune 18 | CCACCCCTAACACTGGTC |
| (SEQ ID NO: 63) | |
| SB9 fine tune 19, | CCCCTAACACTGGTCTTC |
| selected as SB | |
| molecule (SB9-19) | |
| (SEQ ID NO: 64) | |
| SB9 fine tune 20 | CCCTAACACTGGTCTTCT |
| (SEQ ID NO: 65) | |

In some embodiments, ASOs may be further modified. In some embodiments, modifications include conjugation of additional moieties (e.g., lipids) to one of more ASO nucleotides. Furthermore, the ASO may also include one or more chemically modification of one or more nucleobases, nucleotides, or internucleoside linkages.

### ASO Conjugation and Modifications

Additional moieties (e.g., lipid moieties) may be conjugated to any one or more nucleotides within an ASO to increase delivery efficiency, specific cellular or tissue targeting, cellular uptake, and/or prolonged circulation time. In some embodiments, one or more nucleotides within the ASO is conjugated a lipophilic moiety. In some embodiments, the chemical moiety is conjugated to one or more of the nucleotide on the 5′ or 3′ terminal ends of the ASO (i.e., either 5′ and/or 3′ of the DNA gap in a gapmer). In some embodiments, every nucleotide of the ASO is conjugated to a chemical moiety (i.e., a fully 2′MOE configuration). In some embodiments, the first 5′ terminal nucleotide is conjugated to a chemical moiety. In some embodiments, the last 3′ terminal nucleotide is conjugated to a chemical moiety. Representative lipophilic moieties include palmitic acid, sterols (e.g., tocopherol, cholesterol), carbohydrates (e.g., N-Acetylgalactosamine; GalNAc), oleyl residues, retinyl residues, cholesteryl residues, cholic acid, adamantane acetic acid, 1-pyrene butyric acid, dihydrotestosterone, 1,3-Bis-O (hexadecyl)glycerol, geranyloxyhexyl groups, hexadecylglycerol, borneol, menthol, 1,3-propanediol, heptadecyl groups, myristic acid, O3-(oleoyl) lithocholic acid, O3-(oleoyl) cholenic acid, dimethoxytrityl, and phenoxazine.

Representative tocopherols include α-tocopherol, β-tocopherol, γ-tocopherol, and δ-tocopherol. Analogs of a tocopherol include various unsaturated analogs of α-tocotrienol, β-tocotrienol, γ-tocotrienol, and δ-tocotrienol.

In some embodiments, one or more nucleotides within the ASO is conjugated with a lipid, such as palmitic acid, tocopherol, or cholesterol. Lipids may be connected to the ASO's 5′ or 3′ end with a PS (*) or a PO ( ) linkage. In some embodiments, one or more nucleotides within the ASO is conjugated with a lipid. In embodiments where two or more nucleotides are so modified, the lipids may be the same or different. For example, in an embodiment where three nucleotides are conjugated to a lipid, each of the lipids may be different, e.g., palmitic acid, tocopherol, and cholesterol. Additional lipophilic moieties suitable for conjugation to nucleic acids are known in the art. See, e.g., U.S. Pat. Nos. 8,106,022, 8,404,862, 10,077,443, 10,358,643, 10,441,653, 11,116,843, and 11,260,134 and U.S. Patent Application Publications 2014/0045919, 2016/0289677, 2021/0163934, and 2022/0175817.

In some embodiments, the ASO comprises a chemical modification to one or more nucleobases, sugar moieties, internucleoside linkages (i.e., the backbone), or combinations thereof.

Nucleobase modifications include any modification or substitution that is structurally distinguishable from, yet functionally interchangeable with, a nucleobase, including 5-substituted pyrimidines, 6-azapyrimidines, alkyl or alkynyl substituted pyrimidines, alkyl substituted purines, and N-2, N-6 and 0-6 substituted purines. Representative modified nucleobases include pseudouridine, 2′-thiouridine (s2u), N6′-methyladenosine, 5′methylcytidine, N-ethylpiperdine 7′-EAA triazole modified adenine, N-ethylpiperdine 6′-triazole modified adenine, 6′-phenylpyrrolo-cytosine, 2′,4′-difluorotoluyl ribonucleoside, 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine, 2-thiocytosine, 5-propynyl (C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, tricyclic pyrimidines, universal bases, hydrophobic bases, promiscuous bases, size-expanded bases, and fluorinated bases. Representative tricyclic pyrimidines include 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine, and 2-pyridone.

Additional nucleobase modifications are known in the art. See, e.g., U.S. Pat. No. 3,687,808 5,130,302, 5,811,534, 5,830,653, and 6,005,096, U.S. Patent Application Publications 20030158403 and 2003/0175906, and Kroschwitz et al., eds., The Concise Encyclopedia of Polymer Science and Engineering, 1st ed., John Wiley & Sons, 1990.

Sugar moiety modifications include bicyclic, tricyclic, and non-bicyclic sugar moieties or sugar surrogates, including furanosyl sugar moieties (e.g., 2-deoxyfuranosyl sugar moiety modification). Representative modified furanosyl sugar moieties include acyclic modifications for example, at the 2′, 4′, and 5′ positions. In some embodiments, the acyclic modification is branched. Representative modifications to the sugar moiety 2′ position include 2′-deoxy-2′-fluoro (2′-F), 2′-arabino-fluoro (2′-Ara-F), 2′-O-benzyl, 2′-O-methyl-4-pyridine (2′-O-CH₂Py (4)), 2′-O-mehtyl (2′—OCH₃) (2′-OMe or Me), 2′-O-methoxyethyl (2′-O(CH₂)₂OCH₃) (2-O-MOE or MOE), halo, allyl, amino, azido, unlocked nucleic acid (UNA), glycol nucleic acid (GNA), SH, CN, OCN, CF3, OCF3, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ), and OCH₂C(═O)—N(Rₘ)(Rₙ), where each Rₘ and Rₙ are, independently, H, an amino protecting group, or C₁-C₁₀ alky (unmodified or with modifications).

In some embodiments, the sugar moiety modification comprises a fluorine modification at the 2′ position (2′-Fluoro or 2′-F). The 2′-F modification provides high RNA binding affinity and resistance to nuclease degradation. Preparation methods of 2′-F containing sugar moiety modifications are known in the art. See, e.g., U.S. Pat. Nos. 5,459,255 and 6,262,241, U.S. Patent Application Publications 20060036087 and 20110269814, and Ludwig, acta Biochim. Biophys. Acad. Sci. Hung. 16 (3-4): 131-3 (1981) and Ludwig and Eckstein J. Org. Chem. 54:631-635 (1989).

Bicyclic sugar moiety modifications include bridging sugar modifications that form a second ring. In some embodiments, the second ring comprises a bridge between the 4′ and 2′ positions on a furanose ring. In some embodiments, the sugar moiety is a ribose. Representative 4′ to 2′ bridging sugar moiety modifications include(S)-cEt-BNA, tricyclo-DNA (tcDNA), PMO, 4′-CH₂-2′, 4′-(CH₂)₂-2′, 4′-(CH₂)₃-2′, 4′-CH₂—O-2′ (also known as locked nucleic acids or “LNA”), 4′-CH₂—S-2′, 4′-(CH₂)₂-O-2′ (also known as ethylene-bridged nucleic acids or “ENA”), 4′-CH(CH₃)—O-2′ (also known as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”), 4′-C(CH₃)(CH₃)—O-2′, 4′-CH₂—N(OCH₃)-2, 4′-CH₂—O—N(CH₃)-2′, 4′-CH₂—C(H)(CH₃)-2′, 4′-CH₂—C(═CH₂)-2′, 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b}, are, independently, H, a protecting group, or C₁-C₁₂ alkyl (unmodified or with modifications). Additional sugar moiety modifications and analogs thereof are known in the art. See, e.g., U.S. Pat. Nos. 5,859,221, 6,005,087, 6,531,584, 7,399,845, 7,569,686, 7,741,457, 8,022,193, 8,278,283, 8,278,425, 8,278,426, 9,102,938, and 10,119,136, U.S. Patent Application Publication 20100190837, and Zhou et al., J. Org. Chem. 74 (1): 118-34 (2009).

Internucleoside linkage (i.e., backbone) modifications include any altered 3′ to 5′ phosphodiester linkage, including alkylphosphonates (e.g., methoxypropylphosphonate (MOP)) and phosphorothioates (e.g., phosphorothioate (PS)). An ASO comprising multiple modified internucleoside linkages with chiral centers may be stereopure (containing only one stereoisomer), stereorandom (no regularity in stereoisomers), or have a pattern in stereoisomers. Representative internucleoside linkage modifications include phophodiester bonds (P═O) (unmodified, naturally occurring), phosphotriesters, methylphosphonates, phosphoramidates, and phosphorothioates (PS) (P═S) (including Pr isomer and Sp isomer), and phosphorodithioates (PS2) (HS—P═S), 5′-(E)-vinylphosphonate (5′-(E)-VP), 5′-Mmethyl phosphonate (5′-MP), (S)-5′-C-methyl with phosphate, and 5′-phosphorothioate (5′-PS). Non-phosphorus containing internucleoside linkages include, methylenemethylimino (—CH₂—N(CH₃)—O—CH₂), thiodiester, thionocarbamate (—O—C(═O)(NH)—S—), siloxane (—O—SiH₂—O—), peptide nucleic acid (PNA), and N,N′-dimethylhydrazine (—CH₂—N(CH₃)—N(CH₃)—). Neutral charge internucleoside linkages include, phosphotriesters, methylphosphonates (MP), MMI (3′-CH₂—N(CH₃)—O-5′), amide-3 (3′-CH₂—C(—O)—N(H)-5′), amide-4 (3′-CH₂—N(H)—C(═O)—5′), formacetyl (3′-O—CH₂—O-5′), methoxypropyl, and thioformacetal (3′-S—CH₂—O-5′). Preparation methods of phosphorous-containing and non-phosphorous-containing internucleoside linkages are known in the art. See, e.g., U.S. Patent Application Publication 20220049248 and Hu et al., Signal Transduct. Target. Ther. 5 (1): 101-25 (2020).

A 2′-deoxyfuranosyl sugar moiety modification involves the addition of a five-member carbon furanosyl ring sugar moiety having two hydrogens at the 2′-position and may be unmodified or further modified at positions other than the 2′-position. A 2′-O-methoxyethyl sugar moiety modification involves a substitution of the 2′—OH group of a ribosyl ring with a 2′-O(CH₂)₂—OCH₃).

### Pharmaceutical Compositions

Pharmaceutical compositions of the disclosure include a therapeutically effective amount of an ASO and a pharmaceutically acceptable carrier. The term “therapeutically effective amount of an ASO” as used herein refers to a sufficient amount of an ASO to provide the desired therapeutic effect.

The effective amount of an ASO for a given patient varies depending one or more factors that may include the age, body weight, type, location, and severity of the cancer and general health of the subject. Ultimately, the attending physician will decide the appropriate dose and dosage regimen. Typically, the ASO will be given in a series of doses, typically a single dose a week for a number of weeks. In some embodiments, the effective amount of the ASO is about 0.03 mg to about 3 kg per dose. In some embodiments, the effective amount of the ASO is about 0.3 mg to about 300 mg per dose. In some embodiments, the effective dose of the ASO is about 30 mg per dose. In some embodiments, the effective amount of the ASO is about 10 mg/kg subject body weight to about 50 mg/kg subject body weight per dose. In some embodiments, the ASO is administered once a week for about 30 weeks to about 60 weeks. In some embodiments, the ASO is administered once a week for about 40 weeks. In some embodiments, the ASO is administered once a week, twice a week, or every other weekday (e.g., 3 days a week) for about 4 weeks.

Compositions may be provided as sterile liquid preparations, e.g., isotonic aqueous solutions, suspensions, emulsions, dispersions, or viscous compositions, which may be buffered to a selected pH. Liquid, pharmaceutically acceptable carriers include aqueous or non-aqueous carriers alike. The phrase “pharmaceutically acceptable carrier” is art recognized and includes a pharmaceutically acceptable material, composition, or vehicle, suitable for administering ASOs of the present disclosure to mammals. The carriers include liquid or solid filler, diluent, excipient, solvent, or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Representative examples of liquid carriers include water, saline, phosphate buffered saline, and suitable mixtures thereof. The compositions are typically isotonic, i.e., they have the same osmotic pressure as blood. Sodium chloride, potassium chloride, sodium bicarbonate, sodium carbonate, monobasic sodium phosphate monohydrate, anhydrous sodium phosphate dibasic, potassium phosphate monobasic, dibasic sodium phosphate heptahydrate, and isotonic electrolyte solutions (e.g., Plasma-Lyte®) may be used to achieve the desired isotonicity. Hydrochloric acid, and/or sodium hydroxide may be used to adjust the pH of the composition. In some embodiments, the pH may be in the range of about 7.5 to about 8.5. Depending on the carrier and the ASO, other excipients may be added, e.g., wetting, dispersing, or emulsifying agents, gelling and viscosity enhancing agents, preservatives and the like as known in the art.

In some embodiments, the compositions include a pharmaceutically acceptable carrier. The carrier may be lipid-based, e.g., fatty acids, lipid nanoparticles (LNPs), liposomes, lipid vesicles, or lipoplexes. In some embodiments, the ASOs are emulsified in a fatty acid carrier. Representative fatty acids include ethyl eicosapentaenoate (EPA-E), ethyl octadecatetraenoate (ODTA-E), ethyl nonadecapentaenoate (NDPA-E), ethyl arachidonate (AA-E), ethyl eicosatetraenoate (ETA-E), and ethyl heneicosapentaenoate (HPA-E).

In some embodiments, the carrier is an LNP. In certain embodiments, an LNP includes two or more concentric bilayers separated by aqueous compartments. Lipid bilayers may be functionalized and/or crosslinked to one another. Lipid bilayers may include one or more ligands, proteins, or channels.

Lipid carriers, e.g., LNPs may include one or more cationic/ionizable lipids, one or more polymer conjugated lipids, one or more structural lipids, and/or one or more phospholipids. A “cationic lipid” refers to positively charged lipid or a lipid capable of holding a positive charge. Cationic lipids include one or more amine group(s) which bear the positive charge, depending on pH. A “polymer conjugated lipid” refers to a lipid with a conjugated polymer portion. Polymer conjugated lipids include a pegylated lipids, which are lipids conjugated to polyethylene glycol. A “structure lipid” refers to a non-cationic lipid that does not have a net charge at physiological pH. Exemplary structural lipids include cholesterol, fecosterol, sitosterol, ergosterol, campesterol and the like. A “phospholipid” refers to lipids that have a triester of glycerol with two fatty acids and one phosphate ion. Phospholipids in LNPs assemble the lipids into one or more lipid bilayers. LNPs, their method of preparation, formulation, and delivery are disclosed in, e.g., U.S. Patent Application Publication Nos. 2004/0142025, 2007/0042031, and 2020/0237679 and U.S. Pat. Nos. 9,364,435, 9,518,272, 10,022,435, and 11,191,849.

Lipoplexes, liposomes, and lipid nanoparticles may include a combination of lipid molecules, e.g., a cationic lipid, a neutral lipid, an anionic lipid, polypeptide-lipid conjugates, and other stabilization components. Representative stabilization components include antioxidants, surfactants, and salts. Compositions and preparation methods of lipoplexes, liposomes, and lipid nanoparticles are known in the art. See, e.g., U.S. Pat. Nos. 8,058,069, 8,969,353, 9,682,139, 10,238,754, U.S. Patent Application Publications 2005/0064026 and 2018/0291086, and Lasic, Trends Biotechnol. 16 (7): 307-21 (1998), Lasic et al., FEBS Lett. 312 (2-3): 255-8 (1992), and Drummond et al., Pharmacol. Rev. 51 (4): 691-743 (1999).

### Pharmaceutical Kits

In one aspect, the present disclosure is directed to pharmaceutical kits or systems containing one or more ASOs. Kits or systems include a package such as a box, carton, tube, or the like, having in close confinement therein one or more containers, such as vials, tubes, ampoules, or bottles, which contain an ASO or a pharmaceutical composition thereof. The kits or systems may also include printed instructions for using the ASO and pharmaceutical compositions thereof.

In some embodiments, a kit contains a therapeutically effective amount of an anti-MIR17HG pre-RNA ASO, and printed instructions for using same in the treatment of a MIR17HG pre-RNA-contributed disease in a subject. In some embodiments, the kit also contains a MYC proto-oncogene, bHLH transcription factor (MYC) inhibitor and printed instructions on the use of the MYC inhibitor, where the MIR17HG pre-RNA ASO and MYC inhibitor or in the same dosage forms or different dosage forms that are disposed in the same or different containers.

### Method of Use

In one aspect, the present disclosure is directed to treating a subject having a disease in which aberrant expression and function of the MIR17HG pre-RNA plays a role. The method entails administering to a subject in need thereof a pharmaceutical composition that contains a therapeutically effective amount of an ASO.

The term “disease in which aberrant expression and function of the MIR17HG pre-RNA plays a role” is used herein to refer to a disease which may be improved by the therapeutic targeting of the MIR17HG pre-RNA transcript.

The term “treatment” as used herein refers is an approach for obtaining beneficial or desired results, including clinical results. Such results may include one or more of alleviation or amelioration of one or more symptoms of a disease in which MIR17HG pre-RNA plays a role, diminishment of extent of the disease, stabilization of the state of the disease, delay or slowing of the disease, amelioration or palliation of the disease, and remission of the disease (whether partial or total), whether detectable or undetectable.

The term “subject” (or “patient”) as used herein includes all members of the animal kingdom prone to or suffering from a disease in which aberrant expression and function of the MIR17HG pre-RNA plays a role. In some embodiments, the subject is a human. Therefore, a subject “having a disease in which aberrant expression and function of MIR 17HG pre-RNA plays a role,” “having a neoplasm,” or “in need of” treatment according to the present disclosure broadly embraces subjects who have been positively diagnosed, including subjects having active disease who may have been previously treated with one or more rounds of therapy, and subjects who are not currently being treated (e.g., in remission) but who might still be at risk of relapse, and subjects who have not been positively diagnosed but who are predisposed to diseases in which aberrant expression and function of MIR17HG pre-RNA plays a role (e.g., on account of the basis of prior medical history and/or family medical history, or who otherwise present with a one or more risk factors such that a medical professional might reasonably suspect that the subject was predisposed to disease in which aberrant expression and function of MIR17HG pre-RNA plays a role).

Diseases in which MIR17HG pre-RNA plays a role include, for example, neoplasia (cancer), and non-cancerous diseases such as liver diseases.

The term “neoplasia” as used herein is meant a disease characterized by excess proliferation or reduced apoptosis. Illustrative neoplasms for which the disclosure can be used include, but are not limited to pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), multiple myeloma, Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In some embodiments, the disease in which aberrant expression and function of MIR17HG pre-RNA plays a role is multiple myeloma, lymphoma, or colorectal cancer. Representative lymphomas include chronic lymphocytic leukemia, cutaneous b-cell lymphoma, cutaneous t-cell lymphoma, Hodgkin's lymphoma, non-Hodgkin's lymphoma, and waldenstrom macroglobulinemia. Representative colorectal cancers include gastrointestinal tract adenocarcinoma, rectal adenocarcinoma, and colon adenocarcinoma). Multiple myeloma (MM) is a genetically complex malignancy of plasma cells that accounts for about the 10% of hematologic cancers and despite recent advancements, MM remains largely incurable (Gulla and Anderson, Haematologica 105:2358-2367 (2020)).

In some embodiments, the disease in which MIR17HG pre-RNA plays a role is nonalcoholic fatty liver disease (NAFLD), nonalcoholic steatohepatitis (NASH), liver fibrosis, viral hepatitis, or alcoholic liver disease (ALD). In these diseases, MYC, a highly pleiotropic transcription factor that regulates hepatic cell function, has been identified as dysregulated. Overexpression of MYC alters a wide range of roles including cell proliferation, growth, metabolism, DNA replication, cell cycle progression, cell adhesion and differentiation. Overexpressed MYC is often seen in patients with liver fibrosis, as described in more detail in Zheng et al., Genes (Basel) 8:123-20 (2017).

In some embodiments, the disease is one in which a transcript of the MIR 17HG pre-RNA further interacts with MYC proto-oncogene, bHLH transcription factor (MYC) or MYC-binding partners or is characterized by dysregulated MYC or dysregulated MYC-binding partners. Representative diseases in which MYC plays a role include multiple myeloma, B-cell lymphomas (e.g., diffuse large B-cell lymphoma (DLBCL), follicular lymphoma, and burkitt lymphoma), triple negative breast cancer, pancreatic cancer, liver cancer, and gastric cancer.

### Combination Therapy

The therapies of the present disclosure may be used in combination with at least one other active agent in treating diseases and disorders. The term “in combination” in this context means that the agents are co-administered, which includes substantially contemporaneous administration, by the same or separate dosage forms, or sequentially, e.g., as part of the same treatment regimen or by way of successive treatment regimens. Thus, if given sequentially, at the onset of administration of the second therapy, the first of the two therapies is, in some cases, still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

The dosage of the additional therapeutic may be the same or even lower than known or recommended doses. See, Hardman et al., eds., Goodman & Gilman's The Pharmacological Basis of Therapeutics, 10th ed., McGraw-Hill, New York, 2001; Physician's Desk Reference 60th ed., 2006. Anti-cancer agents that may be used in combination with the inventive therapies are known in the art. See, e.g., U.S. Pat. No. 9,101,622 (Section 5.2 thereof). An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these additional active agents would be provided in a combined amount effective to kill or inhibit proliferation of diseased or cancerous cells. This process may involve contacting the cells with recipient cells and the agent(s) or multiple factor(s) at the same time. This may be achieved by contacting the cells with a single composition or pharmacological formulation that includes both agents, or by contacting the cells with two distinct compositions or formulations, at the same time, wherein one composition includes an ASO and the other includes the second agent(s).

In some embodiments, the therapies of the present disclosure are used in conjunction with chemotherapeutic, radiotherapeutic, immunotherapeutic intervention, targeted therapy, pro-apoptotic therapy, or cell cycle regulation therapy.

In some embodiments, the therapies of the present disclosure may precede or follow the additional agent (e.g., anti-cancer) treatment by intervals ranging from minutes to weeks. In embodiments where the additional agent and therapies of the present disclosure are applied separately to the subject, one would generally ensure that a significant period of time did not expire between the times of each delivery, such that the agent and inventive therapies would still be able to exert an advantageously combined effect on the subject's disease. In such instances, it is contemplated that one may administer the subject with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In some embodiments, the therapies of the present disclosure and the additional agent may be administered within the same patient visit; in other embodiments, the two agents are administered during different patient visits.

In some embodiments, the therapies of the disclosure and the additional agent are cyclically administered. Cycling therapy involves the administration of one therapeutic for a period of time, followed by the administration of a second therapeutic for a period of time and repeating this sequential administration, i.e., the cycle, in order to reduce the development of resistance to one or both of the additional therapeutics, to avoid or reduce the side effects of one or both of the additional therapeutics, and/or to improve the efficacy of the therapies. In one example, cycling therapy involves the administration of a first additional therapeutic for a period of time, followed by the administration of a second additional therapeutic for a period of time, optionally, followed by the administration of a third additional therapeutic for a period of time and so forth, and repeating this sequential administration, i.e., the cycle. It is expected that the treatment cycles would be repeated as necessary. It also is contemplated that various standard therapies, as well as surgical intervention, may be applied in combination with the cells of the present disclosure.

Representative types of additional therapeutics are described below. In some embodiments, the additional therapeutic is a MYC inhibitor. Representative MYC inhibitors include cisplatin, gemcitabine, axitinib, nadroparin, and benzamidine.

In some embodiments, the additional therapeutic is an acetyl-CoA carboxylase-a (ACC1) inhibitor. In some embodiments, the AAC1 inhibitor is 5-tetradecyl-oxy-2-furoic acid (TOFA).

In some embodiments, the additional therapeutic is an Immunomodulatory imide drug (IMiD). Representative IMiDs include thalidomide, lenalidomide, pomalidomide, and iberdomide. In some embodiments, the additional therapeutic is a proteasome inhibitor. Representative proteasome inhibitors include bortezomib, carfilzomib (Kyprolis®), delanzomib, ixazomib, marizomib, and oprozomib.

Multiple myeloma therapeutics that may be suitable for the combination with the inventive therapies described herein include belantamab mafodotin-blmf (Blenrep®), bortezomib (Velcade®), carfilzomib (Kyprolis®), carmustine (BiCNU®), ciltacabtagene autoleucel (Carvykti®), cyclophosphamide, daratumumab (Darzalex®), daratumumab and hyaluronidase-fihj (Darzalex Faspro®), doxorubicin hydrochloride liposome (Doxil®), elotuzumab (Empliciti®), idecabtagene vicleucel (Abecma®), isatuximab-irfc (Sarclisa®), ixazomib citrate (Ninlaro®), lenalidomide (Revlimid), melphalan and melphalan hydrochloride (Alkeran® Tablets, Alkeran® for injection, Evomela®), pamidronate disodium (Aredia®), plerixafor (Mozobil®), pomalidomide (Pomalyst®), Selinexor (Xpovio®), thalidomide (Thalomid®), zoledronic acid (Zometa®), and the PAD combination of bortezomib (PS-341), doxorubicin hydrochloride (Adriamycin®), and dexamethasone.

### Immunotherapy

Immunotherapy, including immune checkpoint inhibitors may be employed to treat a diagnosed cancer. Immune checkpoint molecules include, for example, PD1, CTLA4, KIR, TIGIT, TIM-3, LAG-3, BTLA, VISTA, CD47, and NKG2A. Clinically available examples of immune checkpoint inhibitors include durvalumab (Imfinzi®), atezolizumab (Tecentriq®), and avelumab (Bavencio®). Clinically available examples of PD1 inhibitors include nivolumab (Opdivo®), pembrolizumab (Keytruda®), and cemiplimab (Libtayo®).

### Chemotherapy

Anti-cancer therapies also include a variety of combination therapies with both chemical and radiation-based treatments. Combination chemotherapies include, for example, Abraxane®, altretamine, docetaxel, Herceptin®, methotrexate, Novantrone®, Zoladex®, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, Taxol®, gemcitabien, Navelbine®, farnesyl-protein tansferase inhibitors, transplatinum, 5-fluorouracil, vincristine, vinblastine and methotrexate, or any analog or derivative variant of the foregoing and also combinations thereof.

### Radiotherapy

Anti-cancer therapies also include radiation-based, DNA-damaging treatments. Combination radiotherapies include what are commonly known as gamma-rays, X-rays, and/or the directed delivery of radioisotopes to tumor cells which cause a broad range of damage on DNA, on the replication and repair of DNA, and on the assembly and maintenance of chromosomes. Dosage ranges for radioisotopes vary widely, and depend on the half-life of the isotope, the strength and type of radiation emitted, and the uptake by the neoplastic cells and will be determined by the attending physician.

Radiotherapy may include external or internal radiation therapy. External radiation therapy involves a radiation source outside the subject's body and sending the radiation toward the area of the cancer within the body. Internal radiation therapy uses a radioactive substance sealed in needles, seeds, wires, or catheters that are placed directly into or near the cancer.

These and other aspects of the present disclosure will be further appreciated upon consideration of the following working examples, which are intended to illustrate certain embodiments of the disclosure but are not intended to limit its scope, as defined by the claims.

### EXAMPLES

### Example 1: Materials and Methods

Cell lines: Cell lines (CLs) were grown at 37° C., 5% CO2. a) MM-CLs: AMO1, NCI-H929, SK-MM-1, U266, JJN3 and KMS-12-BM were purchased from DSMZ (Braunschweig, Germany). MM.IS, MM.IR and RPMI-8226 were purchased from ATCC (Manassas, VA, USA). ABZB CL is AMO1 bortezomib-resistant and ACFZ Cl is AMO1 carfilzomib-resistant (Morelli et al., Blood 132:1050-1063 (2018)). LR7 CL is U266 melphalan-resistant (Morelli et al., Blood 132:1050-1063 (2018)). These cells were cultured in RPMI-1640 medium (Gibco® Life Technologies, Carlsbad, CA, USA) supplemented with 10% fetal bovine serum (Lonza Group Ltd., Basel, Switzerland) and 1% penicillin/streptomycin (Gibco®, Life Technologies). b) B-cell lymphoma cell lines (BCLOCL): Maver-1, Jeko-1 (mantle cell lymphoma), Sultan, P3HR1, Daudi and Raji (Burkitt lymphoma) (purchased from ATCC) were cultured in RPMI-1640 medium (Gibco® Life Technologies) supplemented with 10% fetal bovine serum (Lonza Group Ltd.) and 1% penicillin/streptomycin (Gibco®, Life Technologies). c) Non-malignant cell lines: HK-2 (human kidney cells, cortex/proximal tubule) were purchased from ATCC and cultured in K-SFM (Keratinocyte Serum Free Medium) (Thermo Fisher Scientific, Waltham, MA, USA). supplemented in accordance with ATCC guidelines; THLE-2 (human liver cells) were purchased from ATCC and cultured in BEGM (Bronchial epithelial cell growth medium) (Lonza Group Ltd.) supplemented in accordance with ATCC guidelines; d) Lenti-X™ 293T (human embryonic kidney, purchased from Takara (cat. no. 632180)) and Flp-In T-REx cells were cultured in DMEM (Dulbecco's modified Eagle's medium) (Gibco®, Life Technologies) supplemented with 10% fetal bovine serum (Lonza Group Ltd.) and 1% penicillin/streptomycin (Gibco®, Life Technologies). e) P493-6 were cultured in RPMI-1640 medium (Gibco® Life Technologies) supplemented with 10% fetal bovine serum (Lonza Group Ltd.) and 1% penicillin/streptomycin (Gibco®, Life Technologies). f) 5TGMI murine MM cells were cultured in IMDM (Iscove modified Dulbecco medium) (Gibco®, Life Technologies) supplemented with 10% fetal bovine serum (Lonza Group Ltd.) and 1% penicillin/streptomycin (Gibco®, Life Technologies). g) Colorectal cancer cell lines HCT116 and DLD-1, parental and DICER mutants, were purchased from Horizon Discovery and cultured in ATCC-formulated McCoy's 5a Medium Modified (Catalog No. 30-2007) supplemented with 10% fetal bovine serum (Lonza Group Ltd.) and 1% penicillin/streptomycin (Gibco®, Life Technologies). Cells were periodically tested to exclude mycoplasma contamination. Cells were STR (short tandem repeats) authenticated.

Primary Patient Cells: Following informed consent approved by the Dana-Farber Cancer Institute Institutional Review Board, CD138+ cells were isolated from the BM aspirates of MM patients by Ficoll-Hypaque (Lonza Group, Basel, Switzerland) density gradient sedimentation; followed by antibody-mediated positive selection using anti-CD138 magnetic activated cell separation microbeads (Miltenyi Biotech, Gladbach, Germany). Purity of immunoselected cells was assessed by flow-cytometry analysis using a phycoerythrin-conjugated CD138 monoclonal antibody by standard procedures. For long-term culture (6 days), CD138+ cells were cultured physically separated from HS-5 cells by means of Falcon Cell Culture Inserts (Corning, New York, NY, USA), according to manufacturer's instructions, as previously described (Morelli et al., Blood 132:1050-1063 (2018)).

Peripheral blood mononuclear cells: Following informed consent approved by the Dana-Farber Cancer Institute Institutional Review Board, CD138+ cells were isolated from the BM aspirates of MM patients by Ficoll-Hypaque (Lonza Group, Basel, Switzerland) density gradient sedimentation; followed by antibody-mediated positive selection using anti-CD138 magnetic activated cell separation microbeads (Miltenyi Biotech, Gladbach, Germany). Purity of immunoselected cells was assessed by flow-cytometry analysis using a phycoerythrin-conjugated CD138 monoclonal antibody by standard procedures. For long-term culture (6 days), CD138+ cells were cultured physically separated from HS-5 cells by means of Falcon Cell Culture Inserts (Corning, New York, NY, USA), according to manufacturer's instructions, as previously described (Morelli et al., Blood 132:1050-1063 (2018)).

RNA-seq, microarray-based gene expression analysis and microRNA profiling of MM patients. RNA-seq: as primary dataset, previously published RNAseq data from CD138+MM cells from 360 MM patients from IFM/DFCI 20019 clinical trial (NCT01191060) were used (Samur et al., Leukemia 32:2626-2635 (2018)). This dataset was used to assess expression of lncRNAs in newly diagnosed MM patients. Unstranded paired-end RNA sequencing were quantified using quasi-mapping with Salmon. Reference transcripts for GRCh38 transcripts were downloaded from Gencode v24. After QC controls TPM values for genes generated from isoform level TPMs with tximport. All figures were created with R and ggpubr. De-novo assembly for the RNAseq data on IFM cohort was done using TopHat. Gencode v24 GTF file were used as the reference and new isoform annotated by TopHat were identified from the output files. Newly diagnosis (ND) and relapse (R) samples from the continuation of the DFCI/IFM study was used to compare ND-MM and R-MM. Similar to diagnosis only samples these samples were sequenced with pared end sequencing and expression was quantified using the same pipeline explained above. As a secondary dataset, TPM level filtered MMRF CoMMpass data were downloaded from MMRF Research portal. Only samples those were collected from CD138+ selected BM samples at diagnosis were used for analysis.

Microarray-based gene expression analysis: RROL expression level was evaluated in a publicly available dataset (GSE66293) (Lionetti et al., Oncotarget 6:24205-17 (2015)) including 129 newly diagnosed and 12 relapsed MM cases that were profiled by GeneChip Human Gene 1.0 ST array (Affymetrix, Santa Clara, CA, USA) (Todoerti et al., Clin. Cancer. Res. 19:3247-58 (2013)). Normalized and re-annotated expression levels were obtained as described (Todoerti et al., Clin. Cancer. Res. 19:3247-58 (2013)), using Chip Definition Files from BrainArray libraries version 20.0.0 (Dai et al., Nucleic Acids Res. 33: e175-9 (2005)). Differential expression between the two groups was assessed by Wilcoxon runk sum test with continuity correction in R environment (version 4.0.4).

miRNA profiling: miRNA expression data for IFM cohort were generated using Affymetrix GeneChip® miRNA Array 4.0 platform. Affy and oligo packages from Bioconductor was used to normalize the miRNA expression data.

Correlation analysis: Spearman correlation was used to evaluate correlation between lncRNA, mRNAs and miRNAs.

Survival analysis: survival analysis was performed using survival package in R, and log rank test was used to compare groups.

Generation of dCAS9-KRAB cell lines. Cell lines expressing the dCas9-KRAB fusion protein were generated as previously described Morelli et al., Methods Mol. Biol. 2348:189-204 (2021). Briefly, cells were infected with a lentivirus expressing the dCas9-BFP-KRAB transgene (Addgene, Plasmid #46911) and sorted for clones stably expressing high BFP. Infection was performed at low MOI (<0.4). Validation of transcriptional repression in MM cell lines expressing the dCas9-KRAB fusion protein was assessed by infecting lentivirus expressing a sgRNA for ENO1 (gRNA_ENO1: CCGGCGAGATCTCCGTGCTC (SEQ ID NO: 66) or a non-targeting negative control (gRNA_NC: GATGTGGTCATTCGTCATGA (SEQ ID NO: 67). sgRNAs were cloned into pU6-sgRNA EF1Alpha-puro-T2A-BFP (Plasmid #60955). This procedure followed protocols established by Weissman Lab and available online weissmanlab.ucsf.edu/CRISPR/CRISPR.html. Downregulation of ENO1 was assessed by qRT-PCR analysis following procedure described below for Reverse transcription (RT) and quantitative real-time amplification (qRT-PCR).

CRISPRi viability screens. Library design: gRNAs to target lncRNA TSSs were used designed using the Broad Institute web portal (now called CRISPick: portals.broadinstitute.org/gppx/crispick/public). For primary screen, target lncRNAs were selected based on median TPM>0.5 in the IFM/DFCI cohort. For secondary screen, target lncRNAs were selected based on primary screen results (i.e., targeted by significantly depleted or enriched gRNAs, FDR<0.25); plus additional lncRNAs identified through a de-novo assembly of RNA-seq data and manually selected lncRNAs selected based on their impact on the clinical outcome of MM patients enrolled in the IFM/DFCI clinical study.

gRNA pool library production: Primary CRISPRi library consisting of 7,500 gRNAs or secondary CRISPRi library consisting of 3,750 gRNAs were co-transfected with packaging plasmids (psPAX2, Addgene #12260; pMD2.G, Addgene #12259) into HEK293T cells using Lipofectamine 2000 transfection reagent (Thermo Fisher Scientific) following the manufacture's protocol. Library DNA (4 μg), psPAX2 DNA (4 μg) and VSV-G DNA (2 μg) was mixed and transfected into HEK293T cells in a T75 flask (×10). Six hours after transfection, media was removed and replaced with 10 ml of virus production media (DMEM media supplemented with 10% of FBS). Forty-eight hours after transfection, lentiviral media was harvested, concentrated using Lenti-X™ Concentrator (Takara, cat. no. 631232) and stored at −80° C.

Virus titer determination: 1×10⁶ cells (each cell line) were plated per well of a 6-well plate. Cells were infected with different amounts of lentivirus overnight in the presence of 8 μg/ml of polybrene. The titering of lentiviral particles was performed by flow-cytometry following protocol from Cellecta, section 5.3 and 5.4.

Primary screening: 4×10⁷ MM cells expressing dCAS-KRAB fusion protein were infected, using Spinoculation, with library lentiviral particles at MOI ranging from 0.1 to 0.3. Infection was performed in triplicate. Virus-containing media was removed after 1 h of Spinoculation, cells were washed 2× with PBS and cultured in complete media. After 4 days, cells were selected with puromycin for 3 additional days. At day 7, cellular debris were removed by Ficoll-Hypaque (Lonza Group, Basel, Switzerland) density gradient sedimentation. Cells were cultured for additional 2 weeks ensuring a 1000× representation of library. Genomic DNA was isolated using Blood & Cell Culture DNA Maxi/Midi Kit (Qiagen #13362,13343) following the manufacturer's protocol. Cellecta (Mountain View, CA) performed PCR amplification of the gRNA cassette for Illumina sequencing of gRNA representation. Protocols for PCR and Illumina sequencing are available online.

Screening data analysis: For candidate gene discovery, the normalized gRNA count table was loaded into MaGeCK (Model-based Analysis of Genome-wide CRISPR-Cas9 Knockout) by comparing the experimental and control (plasmid library) conditions. Top genes were determined based on mean log 2 fold change (LFC) for all gRNAs and false discovery rate (FDR).

In vitro validation of MIR17HG: Top scoring (n=4, MIR 17HG sgRNAs #1-4, SEQ ID NOs: 2-5) sgRNAs targeting MIR17HG were cloned into a pRSGT16-u6Tet-sg-CMV-TetRep-2A-TagRFP-2A-Puro (Cellecta, cat. #SVCRU6T16-L) vector and confirmed by sequencing. gRNA constructs were co-transfected with packaging plasmids (psPAX2, Addgene #12260; pMD2.G, Addgene #12259) into HEK293T cells using Lipofectamine 2000 transfection reagent (Thermo Fisher Scientific) following the manufacture's protocol. Virus was harvested 48 hours later, concentrated, and stored at −80° C. MM cell lines stably expressing dCas9-KRAB fusion protein were infected with a lentivirus driving expression of individual sgRNAs. Infected cells were selected using puromycin. Expression of sgRNAs was obtained by doxycycline (0.5 μg/mL, every other day).

Antisense oligonucleotides, synthetic miRNA mimics and inhibitors, siRNAs. Long Non-Coding LNA gapmerRs, SEQ ID NOs: 2-14, were custom-designed and purchased from Exiqon (Vedbaek, Denmark).

Synthetic mimics and inhibitors for miR-17a, miR-18a, miR-19a, miR-20a, miR-19b-1 and miR-92al were purchased from Ambion (Applied Biosystems, CA, US). Silencer selected siRNAs were purchased from Ambion (Applied Biosystems, CA, US). Design of t-ASOs is described in Table 2-Table 4.

Gymnosis. Gymonotic experiments were performed as previously described (Taiana et al., Methods Mol. Biol. 2348:157-166 (2021)). Briefly: cells were seeded at plating density to reach confluence on the final day of the experiments. Cell number at plating ranged from 0.5 to 2,5×10³ in 96-well plates, from 2,5 to 10×10⁴ in 12-well plates, from 1 to 3×10⁵ in 6-well plates. For ChIP and Co-IP experiments, cell number at plating was 1×10⁶ in T75 flask (10 mL final volume).

Transient transfection of cells. Cells from adherent cell lines were transfected by Lipofectamine 2000 according to manufacturer instructions with 25 nM of LNA gapmeRs (Exiqon). Cells from suspension cell lines (i.e., non-adherent) were transfected (electroporation) by Neon Transfection System (Invitrogen, CA, US), (2 pulses at 1150, 30 ms). LNA gapmeRs, miRNA inhibitors/mimics and siRNAs were used at 25 nM. The transfection efficiency evaluated by flow-cytometric analysis relative to a FAM dye-labeled anti-miR-negative control reached 85% to 90%.

Stable expression using lentiviral plasmids. To generate cells stably over-expressing miR-17-92 cluster, AMO1 were transduced with PMIRH17-92PA-1 lenti-vector (System Biosciences, Palo Alto, CA, USA). To generate cells stably expressing c-MYC, U266 were transduced with Lenti ORF clone of Human v-myc myelocytomatosis viral oncogene homolog (avian) (MYC), Myc-DDK-tagged (RC201611L3) (Origene Technologies, Rockville, Maryland, MD). To generate cells stably expressing WDR82, AMO1 were transduced with Lenti ORF clone of Human WD repeat domain 82 (WDR82), mGFP tagged (RC216325L4) (Origene Technologies, Rockville, Maryland, MD). To generate cells stably expressing Cas9, AMO1 and H929 were transduced with pLX_311-Cas9 (Addgene #96924). Cells expressing the transgene were selected by antibiotic-selection for 3 to 5 days.

CRISPR/CAS9 gene knockout. To generate DROSHA KO cells, AMO1 and H929 stably expressing Cas9 were transduced with transEDIT CRISPR single gRNA lentiviral expression vectors targeting DROSHA (CMV promoter, ZsGreen, TEVH-1203933) (transOMIC technologies Inc., Huntsville, AL, USA). ZsGreen+ cells were sorted (BD FACSARIA III; BD Biosciences, Qume Drive San Jose, CA, USA) 5 days after infection and cultured.

Cell viability assay. Cell viability was evaluated by Cell Counting Kit-8 (CCK-8) assay (Dojindo Molecular Technologies) and 7-AminoactinoMYCin (7-AAD) flow cytometry assays (BD biosciences), according to manufacturer's instructions. Flow cytometry analysis was performed either by FACS CANTO II (BD biosciences) or by Attune NXT Flow cytometer (Thermo Fisher Scientific).

Detection of apoptosis. Apoptosis was investigated by Annexin V/7-AAD flow cytometry assay (BD biosciences) and by electronic microscopy. Flow cytometry analysis was performed either by FACS CANTO II (BD biosciences) or by Attune NxT Flow cytometer (Thermo Fisher Scientific).

Reverse transcription (RT) and quantitative real-time amplification (qRT-PCR). RNA extraction, reverse transcription (RT) and quantitative real-time amplification (qRT-PCR) were performed as previously described (Morelli et al., Blood 132:1050-1063 (2018)). Briefly, total RNA was extracted from cells with TRIzol® Reagent (Thermo Fisher Scientific), according to manufacturer's instructions. Nuclear and cytosolic subcellular RNA purification was performed using RNA Subcellular Isolation Kit (cat. no. 25501) (Active Motif, Carlsbad, CA), according to manufacturer's instructions. The integrity of total RNA was verified by nanodrop (Celbio Nanodrop Spectrophotometer nd-1000). For RROL (MIR17HG) and mRNA dosage studies, oligo-dT-primed cDNA was obtained through the High-Capacity cDNA Reverse Transcription Kit (Thermo Fisher Scientific) and then used as a template to quantify:
a) human RROL (Hs03295901), ACACA or ACC1 (Hs01046047_m1), ANO6 (Hs03805835_m1), EXT1 (] (Hs00245497_m1), MALATI (Hs00273907_s1) AND PVT1 (Hs00413039_m1). Normalization was performed with human GAPDH (Hs03929097_g1) or ACTB (Hs03023943_g1) or 18S (Hs03003631_g1).
b) murine rrol (Mm01230322_s1), acaca (Mm01304258_m1) and fer (Mm00484303_m1).

Single-tube TaqMan miRNA assay (Thermo Fisher Scientific) was used to detect and quantify miR-17 (002308), miR-18a (002422), miR-19a (000395), miR-20a (000580), miR-19b (000396) and miR-92a-1 (000431), according to the manufacturer's instructions, by the use of ViiA7 RT reader (Thermo Fisher Scientific). Mature miRNAs expression was normalized on RNU44 (Thermo Fisher Scientific, assay Id: Hs03929097_g1). RROL isoforms were also detected by SYBR Green qRT-PCR using the following primers: RROL-1 (Fw, 5′-CCTGCAACTTCCTGGAGAAC (SEQ ID NO: 68); Rev, 5′-GTCTCAAGTGGGCATGATGA (SEQ ID NO: 69)), RROL-2 (Fw, 5′-GACCCTCTTTTAAGTTGGGTG (SEQ ID NO: 70; Rev, 5′-TGGCAAAACATTTTCCTCCT (SEQ ID NO: 71)). Comparative real-time polymerase chain-reaction (RT-PCR) was performed in triplicate, including no-template controls. Relative expression was calculated using the comparative cross threshold (Ct) method.

Western blot analysis. Protein extraction and western blot analysis were performed as previously described. Briefly, cells were lysed in 1× RIPA buffer (Cell Signaling Technology) supplemented with Halt Protease Inhibitor Single-Use cocktail (100×, Thermo Scientific). Whole cells lysates (˜20 μg per lane) were separated using 4-12% Novex Bis-Tris SDS-acrylamide gels (Invitrogen), electro-transferred on Nitrocellulose membranes (Bio-Rad). Extraction of nuclear proteins was performed using the NE-PER™ Nuclear and Cytoplasmic Extraction Reagents (Thermo Fisher, #78833), according to manufacturer's instructions. After electrophoresis the nitrocellulose membranes were blocked and probed over-night with primary antibodies at 4° C., then the membranes were washed 3 times in PBS-Tween and then incubated with a secondary antibody conjugated with horseradish peroxidase for 2 hours at room temperature. Chemiluminescence was detected using Western Blotting Luminol Reagent (sc-2048, Santa Cruz, Dallas, TX, USA).

Primary antibodies: anti-MYC [D84C1] (#5605), anti-WDR82 [D2I3B] (#99715), anti-H3K4me3 [C42D8] (#9751) and anti-Lamin A/C (#2032) antibodies were purchased from Cell Signaling Biotechnology (Danvers, MA). Anti-Drosha antibody [EPR12794] (ab183732) was purchased from Abcam (Cambridge, UK). Anti-MYC [9E10] (sc-40), GAPDH (sc-25778) and β-actin (ab96682) antibodies were purchased from Santa Cruz Biotechnology (Dallas, TX, USA). Monoclonal ANTI-FLAG® M2 antibody (F3165) was purchased from Millipore Sigma (Bedford, MA). Secondary antibodies: Anti-rabbit IgG, HRP-linked Antibody (#7074) and Anti-mouse IgG, HRP-linked Antibody (#7076) were purchased from Cell Signaling Biotechnology (Danvers, MA).

RNA FISH. RNA-FISH experiments were conducted according to established protocols (Raj et al., Nat. Methods 5:877-9 (2008); Shaffer et al., PLOS One 8: e75120-9 (2013)). Cells were plated on coverslips coated with poly-L-lysine and allowed to attach for at least 1 hour. The media was then removed, the cells were washed once with 1×PBS, and then fixed and permeabilized in ice cold 95% methanol/5% acetic acid at 4° C. for 10 minutes. After removing the fixative, cells were washed with Wash Buffer A (20% Stellaris RNA FISH Wash Buffer A, Biosearch Technologies, Inc., SMF-WA1-60; 10% Deionized Formamide, EMD Millipore, S4117; in RNAse-free water, Life Technologies, AM9932) for 5 minutes at room temperature. Cells were then incubated with RNA FISH probes (Stellaris) at a working concentration of 125 nM in Hybridization buffer (90% Stellaris RNA FISH Hybridization Buffer, Biosearch Technologies, SMF-HB1-10; 10% Deionized Formamide) at 37° C. in a humidified chamber in the dark overnight. The next day, cells were washed 3 times for 30 minutes each at 37° C. in the dark with Wash Buffer A. The cells were then incubated for 15 minutes with Wash Buffer A plus 1:1000 Hoescht 33342 (Invitrogen, stock 10 mg/mL) at 37° C., followed by a wash with Wash Buffer B (Biosearch Technologies, SMF-WB1-20) for 5 minutes at room temperature. Coverslips were mounted on slides with Vectashield (VWR 101098-042), and coverslips were sealed with clear nail polish. Z-stack images were acquired on an LSM 880 with Airyscan with an oil-immersion 63× objective and a 2-3× zoom (W.M. Keck Microscopy Facility, MIT), and Airyscan processing was performed using the “Auto” strength feature. Representative images were generated using ImageJ.

Co-immunofluorescence with RNA FISH (Co-IF/FISH). Co-IF/FISH experiments were conducted in a similar fashion to the dual RNA-FISH experiment with the following modifications. After adhering the cells to coverslips, the cells were fixed with 4% PFA (VWR, BT140770) in RNase-free PBS for 10 minutes at room temperature. After washing the cells 3× for 5 minutes with PBS, the cells were permeabilized with ice cold 95% methanol/5% acetic acid at 4° C. for 10 minutes. Cells were then blocked with 4% IgG-free Bovine Serum Albumin (VWR, 102643-516) in PBS for 30 minutes and a primary antibody mixture (1:500 Rabbit anti-c-MYC D84C12 in PBS) was then added to the cells and incubated overnight in a humidified chamber at room temperature. The next day, cells were washed 3× with PBS for 5 minutes at room temperature, and a secondary antibody mixture (1:500 Alexa Fluor 488 Goat anti-rabbit IgG, ThermoFisher A11008 in PBS) was added and incubated for 1 hour at room temperature in the dark. Cells were washed 3× with PBS for 5 minutes, and prior to RNA FISH, cells with antibody staining were re-fixed with 4% PFA in PBS for 10 minutes at room temperature, followed by 3× washes with PBS. After the antibody staining and fixation, the RNA FISH protocol was conducted as described above, starting with the wash with Wash Buffer A.

Microarray-based gene expression profiling after RROL depletion. Microarray-based analysis of gene expression changes after treatment with ASO1 was performed as previously described (Morelli et al., Blood 132:1050-1063 (2018)).

RNA-seq analysis of AMO1^{DR-KO} after RROL depletion. Total RNA was extracted as described above and submitted to NovaSeq RNAseq analysis followed by VIPER NGS Analysis pipeline (Cornwell et al., BMC Bioinformatics 19:135-14 (2018)). List of differentially expressed genes (DEGs) were applied to the GSEA or IPA software to reveal biological pathways modulated by RROL.

Luciferase reporter assay. Promoter reporter clones for human ACC1 (NM_198834), ANO6 (NM_001025356), CCDC91 (NM_018318), EPT1 (NM_033505), EXT1 (NM_000127), FER (NM_001308028) and ZYG11A (NM_001004339) were cloned into GLuc-ON™ Promoter Reporter Vector (GeneCopoeia, Rockville, MD). Luciferase reporter assay was performed according to manufacturer's instructions.

ChIRP. RROL and LacZ antisense DNA probes were designed using the online probe designer at singlemoleculefish.com. Oligonucleotides were biotinylated at the 3′ end with an 18-carbon spacer arm. AMO1 cells were collected and subjected to ChIRP using the EZ-Magna ChIRP RNA Interactome Kit (Millipore Sigma, Bedford, MA), according to manufacturer's instructions and established protocols (Chu et al., J. Vis. Exp. 61:3912-6 (2012)).

De novo lipogenesis assay. Cells were seeded at 5×10⁵ cells per well in 6-well plates and incubated for 3 days in presence of treatments (ASO1/10058-F4/IPTG or respective controls). Twenty-four (24) hours before the end of treatment, 1 Ci of 1+C-labeled glucose (ARC-0122D) was added to each well. Cells were harvested, washed with cold PBS and collected in glass tubes. Purified lipid extract is obtained by chloroform-methanol based extraction (Bligh and Dyer, Can. J. Biochem. Physiol. 37:911-7 (1959)). Glucose incorporation in cellular lipids was quantitated by photon emission through scintillation counting and normalized to total protein content.

Lipid profiling. Lipids were extracted from MM cells, dried, and stored under argon until analysis. Lipid species were analyzed by liquid chromatography electrospray ionization tandem mass spectrometry (LC-ESI/MS/MS) on a Nexera X2 UHPLC system (Shimadzu) coupled with hybrid triple quadrupole/linear ion trap mass spectrometer (6500+ QTRAP system; AB SCIEX) by Lipometrix, at KU Leuven, Belgium.

Lipid extraction: lipid extraction was performed with 1 N HCl: CH₃OH 1:8 (v/v), 900 μl CHCl₃ and 200 μg/ml of the antioxidant 2,6-di-tert-butyl-4-methylphenol (BHT; Sigma Aldrich). A mixture of deuterium labeled lipids SPLASH® LIPIDOMIX® Mass Spec Standard (#330707, Avanti Polar Lipids) was spiked into the extract mix. The organic fraction was evaporated using a Savant Speedvac spd111v (Thermo Fisher Scientific) at room temperature and the remaining lipid pellet was stored at −20° C. under argon.

Mass spectrometry. Just before mass spectrometry analysis, lipid pellets were reconstituted in 100% ethanol. Lipid species were analyzed by liquid chromatography electrospray ionization tandem mass spectrometry (LC-ESI/MS/MS) on a Nexera X2 UHPLC system (Shimadzu) coupled with hybrid triple quadrupole/linear ion trap mass spectrometer (6500+ QTRAP system; AB SCIEX). Chromatographic separation was performed on a XBridge amide column (150 mm×4.6 mm, 3.5 μm; Waters) maintained at 35° C. using mobile phase A [1 mM ammonium acetate in water-acetonitrile 5:95 (v/v)] and mobile phase B [1 mM ammonium acetate in water-acetonitrile 50:50 (v/v)] in the following gradient: (0-6 min: 0% B>6% B; 6-10 min: 6% B>25% B; 10-11 min: 25% B>98% B; 11-13 min: 98% B>100% B; 13-19 min: 100% B; 19-24 min: 0% B) at a flow rate of 0.7 mL/min which was increased to 1.5 mL/min from 13 minutes onwards. Sphingomyelins, ceramides, dihydroceramides, hexosylceramides and lactosylceramides were measured in positive ion mode with a precursor scan of 184.1, 264.4, 266.4, 264.4 and 264.4 respectively. Triacylglycerides and diacylglycerides were measured in positive ion mode with a neutral loss scan for one of the fatty acyl moieties. Phosphatidylcholine, lysophosphatidylcholine, phosphatidylethanolamine, lysophosphatidylethanolamine, Phosphatidylglycerol, phosphatidylinositol and phosphatidylserine were measured in negative ion mode by fatty acyl fragment ions. Lipid quantification was performed by scheduled multiple reactions monitoring (MRM), the transitions being based on the neutral losses or the typical product ions as described above. The instrument parameters were as follows: Curtain Gas=35 psi; Collision Gas=8 a.u. (medium); IonSpray Voltage=5500 V and −4,500 V; Temperature=550° C.; Ion Source Gas 1=50 psi; Ion Source Gas 2=60 psi; Declustering Potential=60 V and −80 V; Entrance Potential=10 V and −10 V; Collision Cell Exit Potential=15 V and −15 V. The following fatty acyl moieties were taken into account for the lipidomic analysis: 14:0, 14:1, 16:0, 16:1, 16:2, 18:0, 18:1, 18:2, 18:3, 20:0, 20:1, 20:2, 20:3, 20:4, 20:5, 22:0, 22:1, 22:2, 22:4, 22:5 and 22:6 except for TGs which considered: 16:0, 16:1, 18:0, 18:1, 18:2, 18:3, 20:3, 20:4, 20:5, 22:2, 22:3, 22:4, 22:5, 22:6.

Data Analysis. Peak integration was performed with the MultiQuant™ software version 3.0.3. Lipid species signals were corrected for isotopic contributions (calculated with Python Molmass 2019.1.1) and were quantified based on internal standard signals and adheres to the guidelines of the Lipidomics Standards Initiative (LSI) (level 2 type quantification as defined by the LSI).

ChIP-qPCR. ChIP-qPCR was performed as previously described (Fulciniti et al., Cell Rep. 25:3693-3705 (2018)). Briefly, 1×10⁷ cells (AMO1, H929 and U266MYC-, with corresponding treatments) were cross-linked with 1% formaldehyde for 10 minutes at 37° C. The cross-linked chromatin was then extracted, diluted with lysis buffer, and sheared by sonication. The chromatin was divided into equal samples for immunoprecipitation with specific antibodies. The immunoprecipitates were pelleted by centrifugation and incubated at 68° C. to reverse the protein-DNA cross-linking. The DNA was extracted from the elute by the Qiaquick PCR purification kit (QIAGEN). Antibodies used were as follows: endogenous MYC (Cell Signaling Technology, #13987), MYC-DDK (Santa Cruz Biotechnology, 9E10-x), GFP (Abcam, #ab290), H3K4me3 (#ab8580), Normal Rabbit IgG (Cell Signaling Technology, #2729), Normal Mouse IgG (Santa Cruz Biotechnology, sc-2025). A parallel sample of input DNA from the same cells was used as control. ChIP and input DNA were analyzed using SYBR Green real-time PCR analysis (Applied Biosystems). Primers for ChIP-qPCR: ACC1 Fw: TTTCTCTCTTGCAGAGTGAGGTGTGG (SEQ ID NO: 72) and ACC1 Rv: TACAAAGGCACGGAGAGAGCAAGT (SEQ ID NO: 73).

RNA-Protein Pull-Down. RROL transcripts were cloned into a pBlueScript vector and sequence verified. In vitro transcription and biotynilation was performed using AmpliScribe™ T7-Flash™ Biotin-RNA Transcription Kit (Lucigen, cat. no. #ASB71110), according to manufacturer's instructions. Cell nuclear lysates (from 1×10⁷ AMO1 cells) were incubated with biotinylated RNA and streptavidin beads for RNA pull-down incubation, using Pierce™ Magnetic RNA-Protein Pull-Down Kit (Thermo Fisher Scientific, cat. no. #20164), according to manufacturer's instructions. RNA-associated proteins were eluted and analyzed by western blotting.

RNA Yeast 3 Hybrid. Saccharomyces cerevisiae strain YLW3 was transformed with RNA plasmids, using standard protocols. They were tested for viability by spotting on SC plates depleted of uracil (SC-U). Protein plasmids were transformed into the yeast strain Y8800 and grown in SC-plates depleted of tryptophan (SC-W). Yeast strains YLW3 containing the examined RNA plasmid were mated with the Y8800 yeast strains containing the protein plasmid. Mating was performed according to the manufacturer's protocol in YPD media. Diploids carrying both plasmids were selected in SC media depleted of tryptophan and uracil (SD-WU), and dimerization was tested by growth in (SC-WUH) media, also depleted of histidine. The following day the diploids in the SC-WUH media were transferred to solid agar plates containing different levels of 3AT, a competitive inhibitor of the HIS3 gene product, to increase the stringency of the selection. Only the diploids with significant interaction should be able to produce enough histidine for survival. After 1-3 days the growth of the different colonies in the different conditions was examined to seek out the diploids with the strongest interactions.

RIP-qPCR. RNA immunoprecipitation (RIP) experiments were performed using the Magna RIP RNA-binding Protein Immunoprecipitation Kit (Millipore Sigma, cat. no. 17-701), according to manufacturer's instructions. The anti-MYC antibody [Y69] used for RIP was purchased from Abcam (ab32072). Normal Rabbit IgG was purchased from Cell Signaling Technology (cat. no. #2729). The primers used for detecting RROL are listed above.

Co-immunoprecipitation (Co-IP). Protein lysates were obtained from 1×10⁷ cells (AMO1, H929 and U266MYC-, with corresponding treatments). Coimmunoprecipitation was performed using Pierce™ Co-Immunoprecipitation Kit (Thermo Fisher Scientific, cat. no. 26149), according to manufacturer's instructions. IP antibodies used were as follows: anti-MYC antibody [Y69] was purchased from Abcam (ab32072), Anti-FLAG® M2 antibody was purchased from Millipore Sigma (F3165), Normal Rabbit IgG was purchased from Cell Signaling Technology (2729).

Proximity-dependent biotin identification (BioID). BioID was performed as described by Kalkat et al., Mol. Cell 72:836-848 (2018). Briefly, FBA-MYC cells were grown to 60% confluence into T75 flasks prior to transfection with ASO1 (50 nM, using Lipofectamine 2000 as described above) and treatment with 1 mg/mL doxycycline (Millipore Sigma), 1 μM MG132 (Millipore Sigma) and 50 mM biotin (Bio Basic) for 24 hours. Experiments with FBA-MYC cells, exposed to doxycycline, included 16 biological replicates (8 with RROL depletion and 8 without RROL depletion). Negative controls used for the analysis included 6 biological replicates of FBA-MYC cells not exposed to doxycycline. Cells were harvested by scraping and washed three times with 50 mL of PBS prior to flash freezing. Cell pellets were lysed in 1 mL of modified RIPA buffer (1% NP-40, 50 mM Tris-HCl pH 7.5, 150 mM NaCl, 1 mM EDTA, 1 mM EGTA, 0.1% SDS, 1:100 protease inhibitor cocktail (Thermo Fisher Scientific), 0.5% sodium deoxycholate), with 250U of benzonase (Millipore). Lysate was rotated for 1 h at 4° C., sonicated 3×30 s, then centrifuged at 27000 g for 30 min at 4° C. Biotinylated proteins were isolated by affinity purification with 30 mg of washed streptavidin-Sepharose beads (GE) with rotation for 2 h at 4° C. Beads were then washed 7×1 mL 50 mM ammonium bicarbonate (pH 8.0) prior to tryptic digestion.

Mass Spectrometry. Mass Spectrometry analysis of Co-IP and BioID samples was performed at the Taplin Mass Spectrometry Facility (Harvard Medical School, Boston, MA), according to established protocols.

Animal study. 6-week old female immunodeficient NOD.CB17-Prkdescid/NCrCrl (NOD/SCID) mice (Charles River) or NSG mice (Jackson Laboratory) were housed in our animal facility at Dana-Farber Cancer Institute (DFCI). All experiments were performed after approval by the Animal Ethics Committee of the DFCI and performed using institutional guidelines.

AMO1^{DR-KO} xenograft model: AMODR-KO were gymnotically exposed to ASO1 (2.5 μM) or ASO-NC (2.5 μM) for 2 days before subcutaneous injection into SCID NOD mice. The day of injection (day 0), cell viability was assessed by Annexin V/7-AAD flow cytometry assay, confirming no detectable pro-apoptotic activity of ASO-1 at this time point (not shown). For tumor cells injection, cells were resuspended in PBSIX supplemented with ASO1 (5 μM) or ASO-NC (5 μM); and then mixed with equivalent volume of Matrigel (Corning, #354230) reaching a final oligo concentration of 2.5 μM. 5×10⁶ cells were subcutaneously injected per mice (5 mice per group). Tumor sizes were measured by electronic caliper.

AMO1 xenograft model: 5×10⁶ AMO1 cells were subcutaneously injected in NOD SCID mice. As tumor became palpable (˜50 mm), mice were randomized to receive G2-15b*-TO or SB9-19-TO or vehicle (−) as control (3 groups, 5 mice/group). Treatments were administered via I.P. injection, every other day per 2 weeks, at 10 mg/kg. Tumor sizes were measured by electronic caliper. In an independent experiment used for qRT-PCR analysis of RROL and ACC1, mice were enrolled to receive treatment after tumors reached the volume of ˜200 mm and treated at day 1-3-5. Tumors were then collected at day 6.

MOLP8-luc+ xenograft model: 1×10⁶ MOLP8-luc+ cells were injected via tail vein in 28 NSG mice. 3 mice (marked by a X) were then excluded for failed injection. The day after, 11 mice were assigned to the control group, 8 mice for treatment with G2-19b*-TO and 6 mice for treatment with SB9-19-TO. Treatments were administered via I.P. injection, every other day per 2 weeks, at 10 mg/kg. At the end of the treatment cycle (day 15), BLI was measured as indication of tumor growth.

Tumor growth inhibition (% TGI) was determined, as previously described (Buck et al., Cancer Res. 68:8322-32 (2008)), by the formula: % TGI= (1-[Tt/T0/Ct/C0] 1-[C0/Ct])×100 where Tt=median tumor volume of treated at time t, T0=median tumor volume of treated at time 0, Ct=median tumor volume of control at time t and C0=median tumor volume of control at time 0.

Statistical Analysis. All in vitro experiments were repeated at least three times and performed in triplicate; a representative experiment was showed in figures. Statistical significances of differences were determined using Student's t test (unless otherwise specified), with minimal level of significance specified as p<0.05. Kaplan-Meier survival curves were compared by log-rank test. Statistical analyses were determined using GraphPad software. Graphs were obtained using GraphPad software (unless otherwise specified).

### Example 2: A Genome-Wide CRISPRi Viability Screen Identifies MIR 17HG as a Leading Dependency in MM

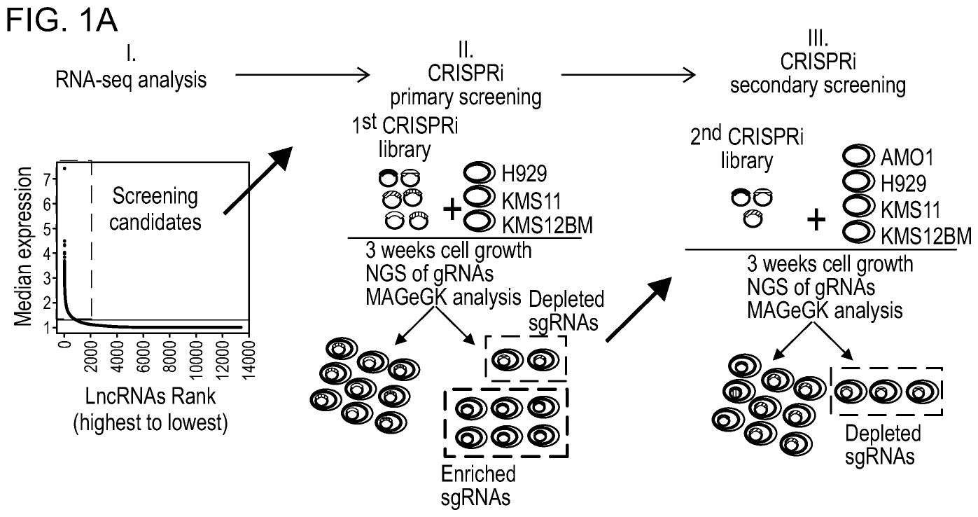

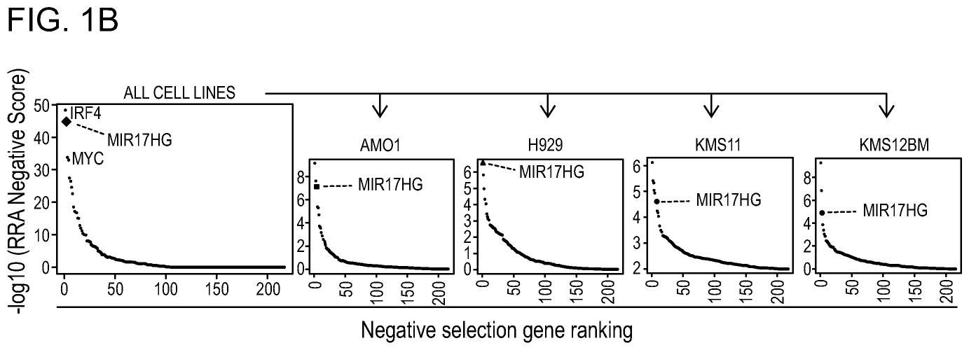

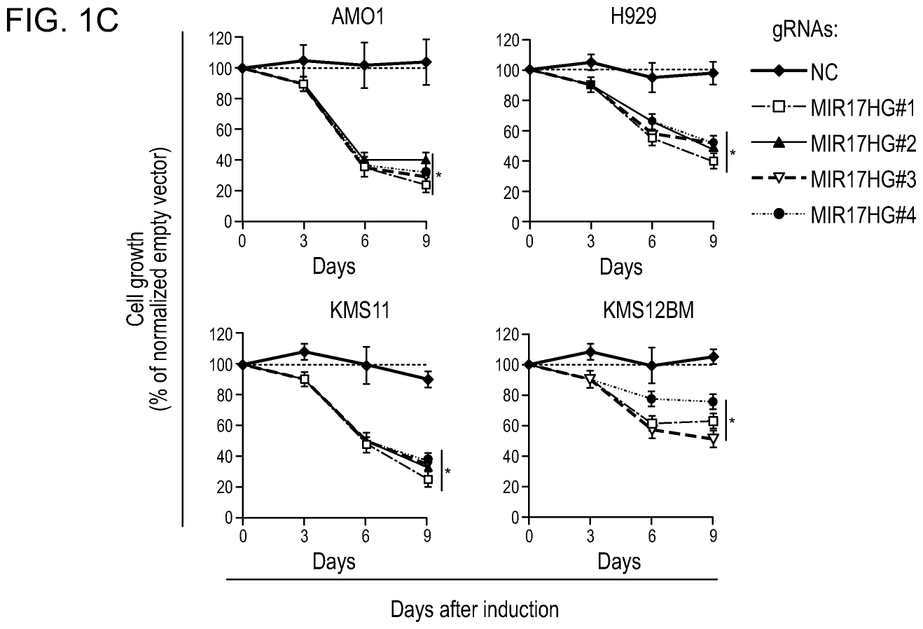

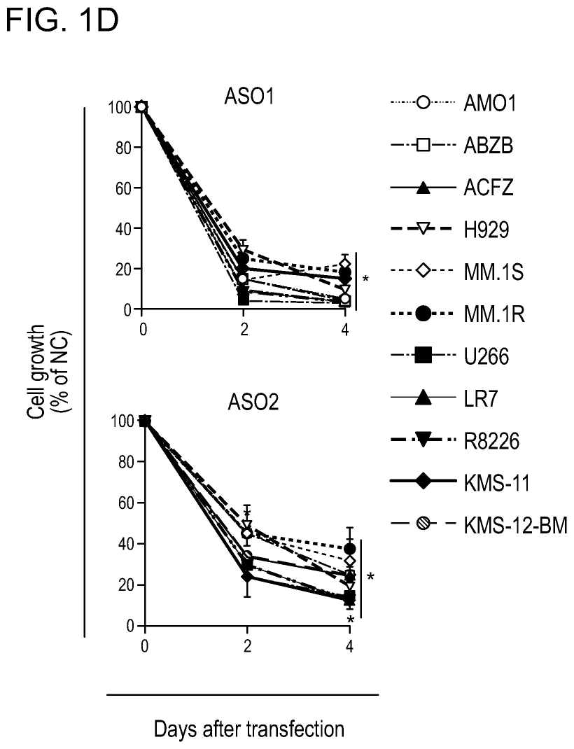

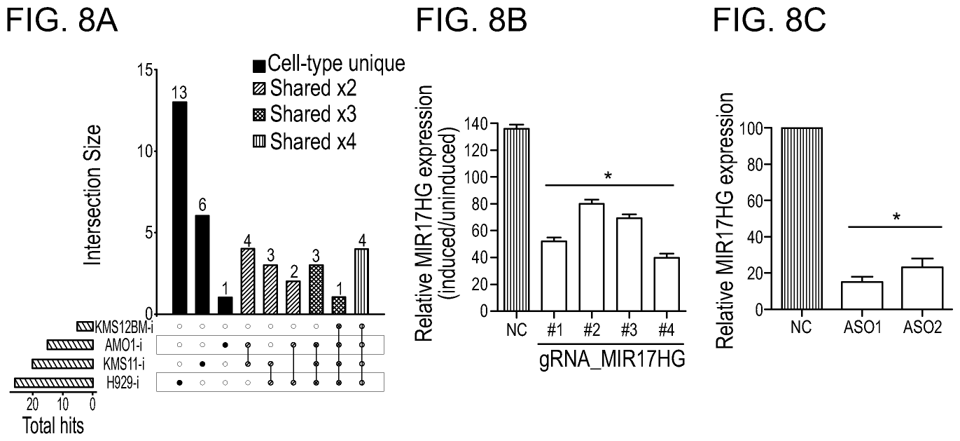

RNA-seq data from 360 newly diagnosed MM patients was analyzed and 913 lncRNA transcripts were identified as expressed in primary MM cells, as illustrated in FIG. 1A left panel labeled I, and in a panel of 70 MM cell lines. To systematically interrogate the role of these lncRNAs in MM cell growth, 3 MM cell lines (H929, KMS-11 and KMS-12-BM) were engineered to express a dCAS9-KRAB fusion protein and transduced with a pooled library consisting of 7 sgRNAs against each of the 913 transcription start sites (TSS) and 576 negative control sgRNAs (see FIG. 1A middle panel labeled II). Relative representation of sgRNAs were assessed by deep sequencing after 3 weeks and analyzed using the Model-based Analysis of Genome-wide CRISPR-Cas9 Knockout (MAGeCK) robust rank aggregation (RRA) algorithm (Li et al., Genome Biol. 15:554-12 (2014)). The most enriched or depleted sgRNAs were further tested in secondary screens using a pooled library targeting 224 lncRNA TSS, the TSS of known protein coding oncogenes (MYC, IRF4) (Chesi et al., Cancer Cell 13:167-80 (2008); Shaffer et al., Nature 454:226-31 (2008)) or tumor suppressors (TP53) (Jovanović et al., Front. Oncol. 8:665-7 (2019)) as positive controls, and 2245 non-targeting sgRNAs as negative control, as illustrated in FIG. 1A, right panel labeled III. In the secondary screens 4 MM cell lines (H929, KMS11, KMS12BM and AMO1) were used to detect and rank significantly depleted or enriched sgRNAs. As expected, sgRNAs targeting IRF4 and MYC were significantly depleted in three (MYC) or all (IRF4) cell lines, while sgRNAs targeting TP53 were significantly enriched in both TP53 wild-type cell lines (AMO1 and H929) (Tessoulin et al., J. Hematol. Oncol. 11:137 (2018)). FIG. 1B highlights the top lncRNA dependency MIR17HG, along with protein coding genes IRF4 and MYC that were used as positive controls. FIG. 1C illustrates the CCK-8 proliferation assay of the MM cell lines AMO1, H929, KMS11 and KMS12BM which stably expressed the KRAB-dCAS9 fusion protein and transduced with lentivectors to conditionally express anti-MIR17HG sgRNAs. A CCK-8 assay was performed at indicated time points after exposure to doxycycline (0.5 μg/mL). Cell proliferation was calculated compared to parental cells infected with the empty sgRNA vector and exposed to doxycycline under some conditions. Focusing on depleted sgRNAs, lncRNA dependencies in MM cells that were either cell type specific (54%) or shared by two or more cell lines (46%) (see, FIG. 8A) were identified. A ranked analysis of sgRNA depletion identified MIR17HG as the leading lncRNA dependency in the screen, with RRA scores equal or superior to those obtained by targeting MYC or IRF4 in all cell lines tested (FIG. 8B). To validate this data further, MM cell lines expressing dCAS9-KRAB fusion protein were transduced with the top four sgRNAs targeting MIR17HG under the regulation of a tetracycline-inducible promoter and observed reduced cell growth compared to cells infected with non-targeting sgRNAs after continued exposure to doxycycline, as illustrated in FIG. 1C and FIG. 8B. MIR17HG expression was investigated by qRT-PCR five days after induction of gRNAs with doxycycline. The results shown in FIG. 8B as the average RNA expression levels after normalization with ACTB and ΔΔCt calculations.

Moreover, 2 different locked nucleic acid (LNA) gapmeR ASOs targeting the MIR 17HG nascent RNA (pre-RNA) for RNase H-mediated degradation (Lai et al., Mol. Cell 77:1032-1043 (2020); Lee and Mendell, 2020) were used to transfect 11 MM cell lines including those resistant to conventional anti-MM agents (AMO1-ABZB resistant to bortezomib; AMO1-ACFZ resistant to carfilzomib; MM.IR resistant to dexamethasone); and confirmed significant impact on MM cell viability independent of the genetic and molecular background, as illustrated in FIG. 1D and FIG. 8C. ASOs were used at concentration of nM. Cell viability was measured 2 and 4 days after electroporation, and it is represented as % of viability compared to cells transfected with NC-ASO. Data from 1 out of 3 independent experiments is shown in FIG. 1D and FIG. 1E. Data present mean+s.d.in FIG. 1D and FIG. 1E. * p<0.05 by Student's/test. FIG. 8C shows MIR17HG expression by qRT-PCR 24 h after transfection. The results shown are an of average RNA expression levels after normalization with ACTB and ΔΔCt calculations. One (1) of three independent experiments is shown.

These data establish a broad dependency to lncRNAs in MM cells. The molecular and functional roles of MIR 17HG is further explored in the MM setting.

### Example 3: MIR17HG^{RROL} (RROL) Mediates Dependency in microRNA-Independent Manner

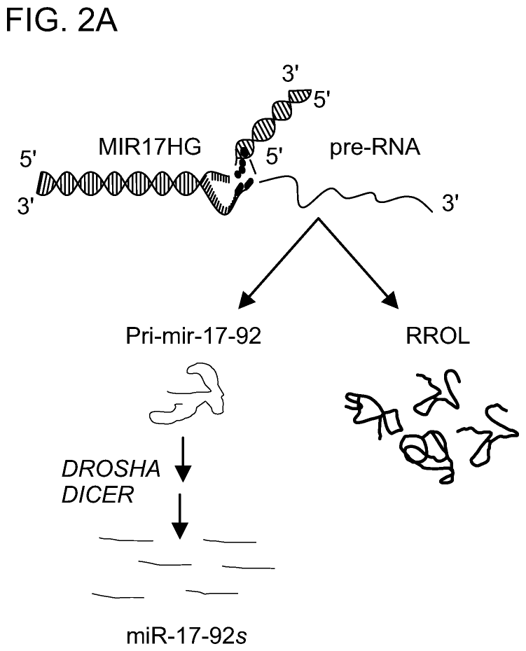

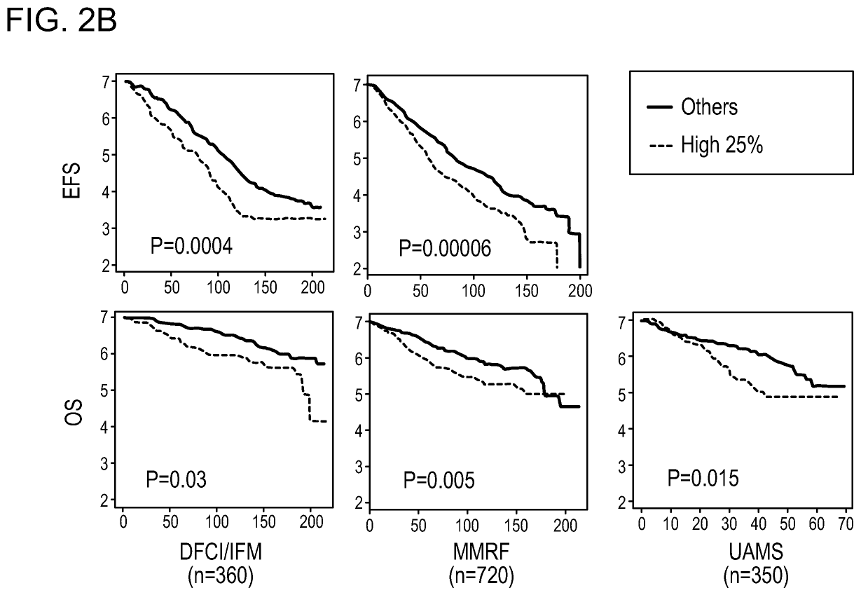

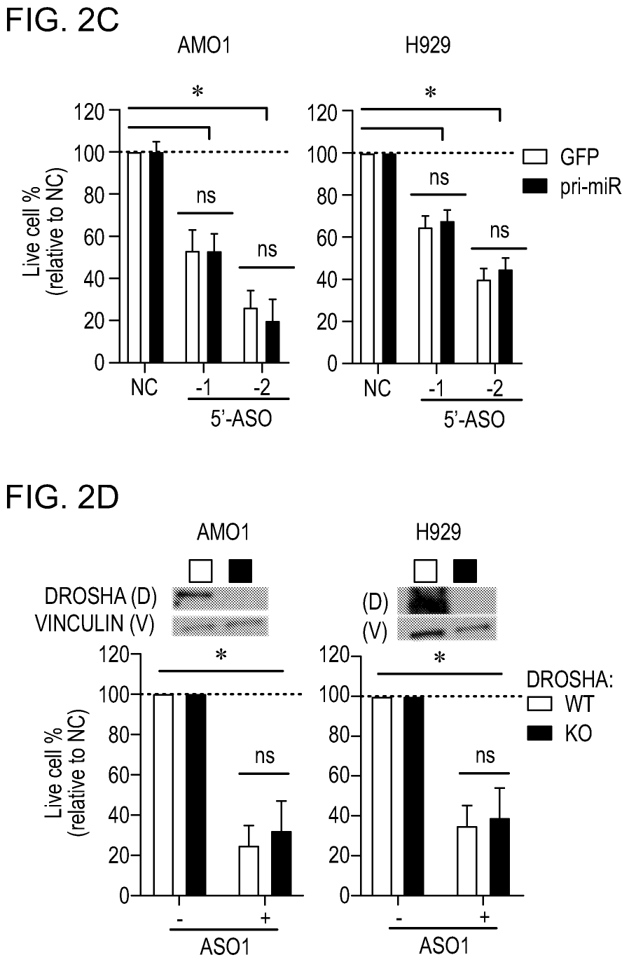

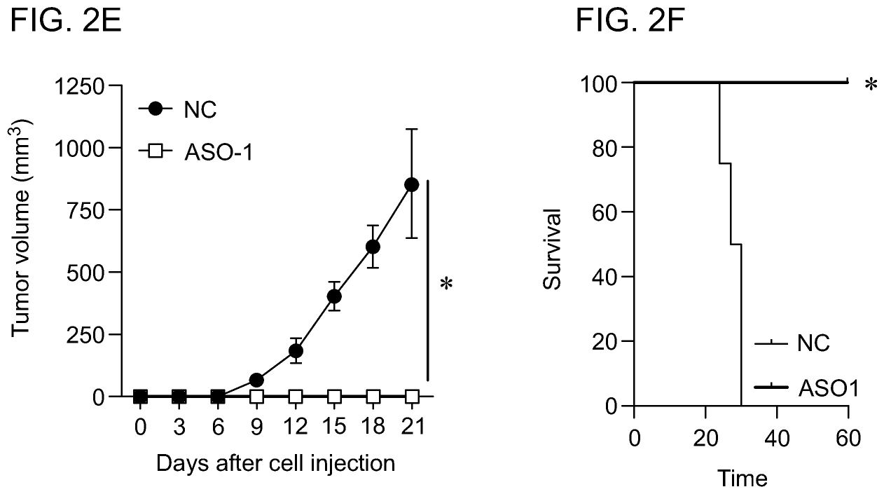

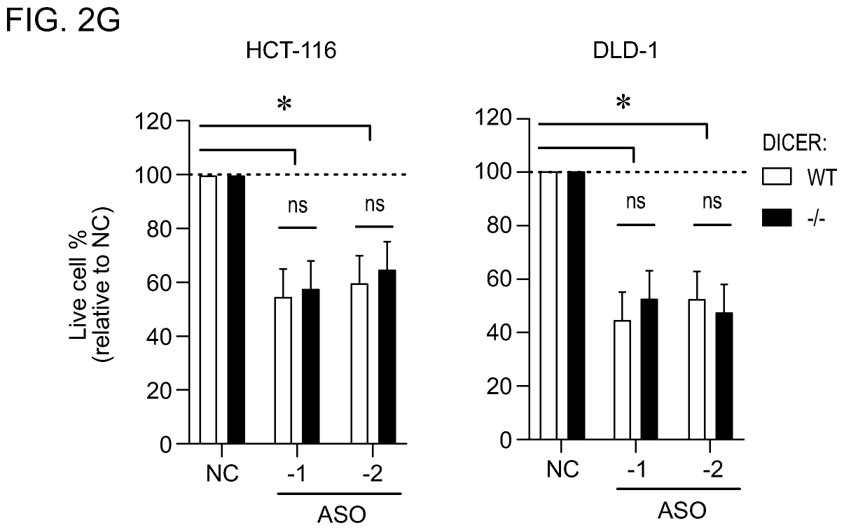

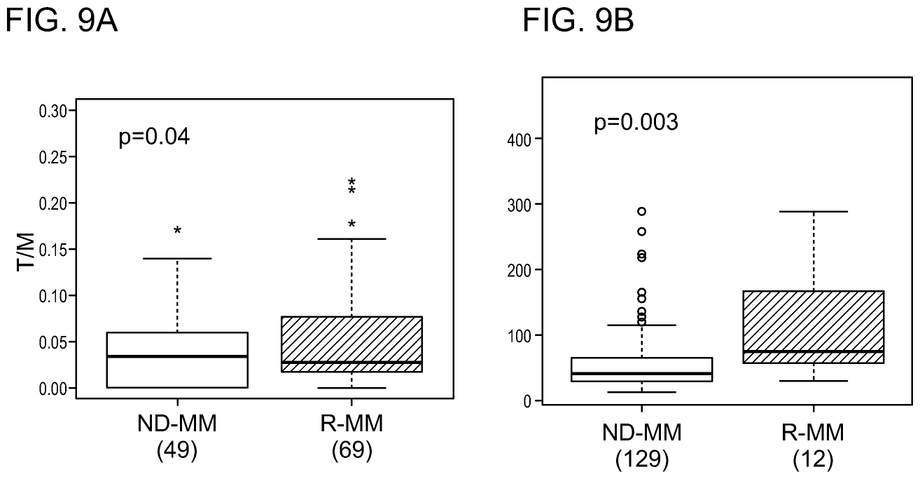

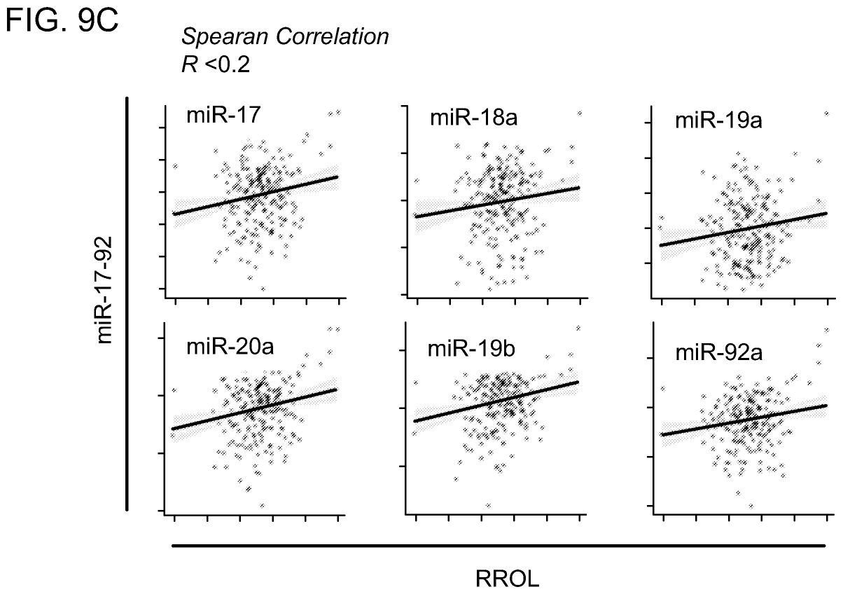

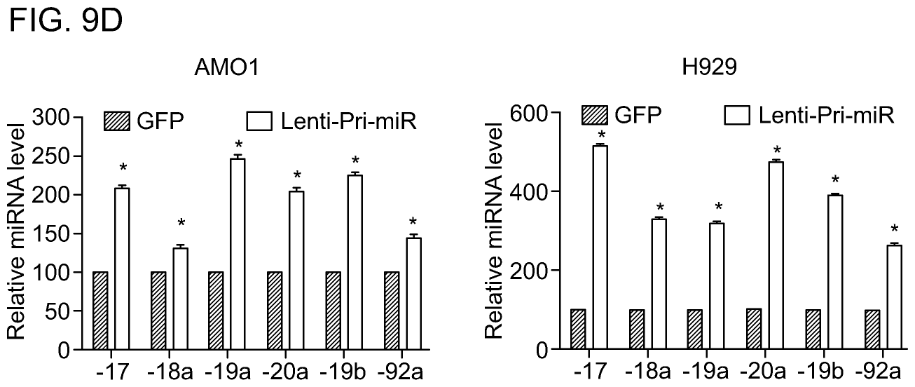

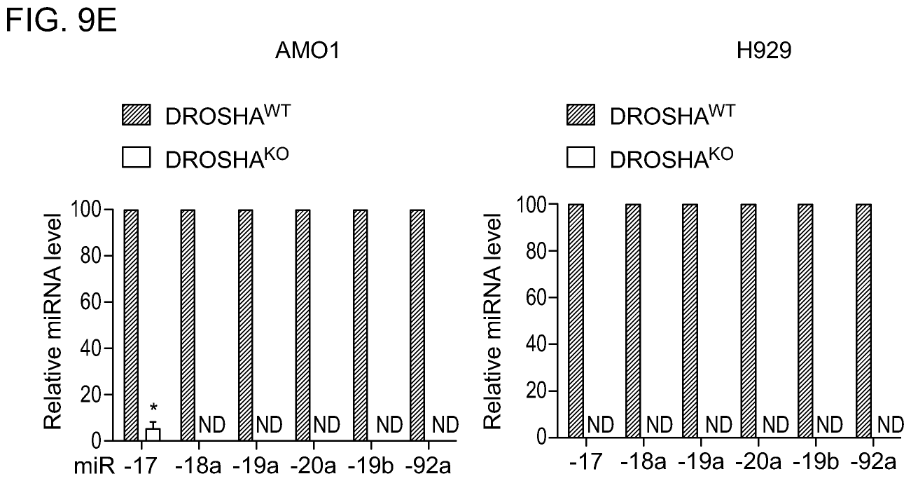

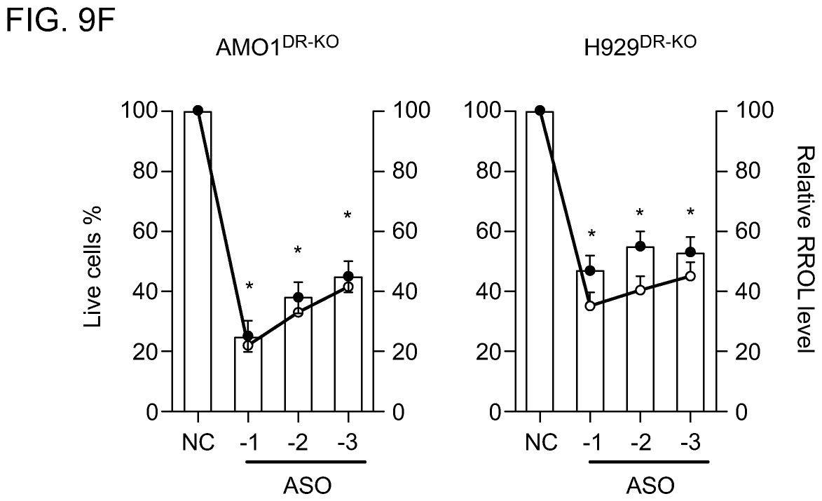

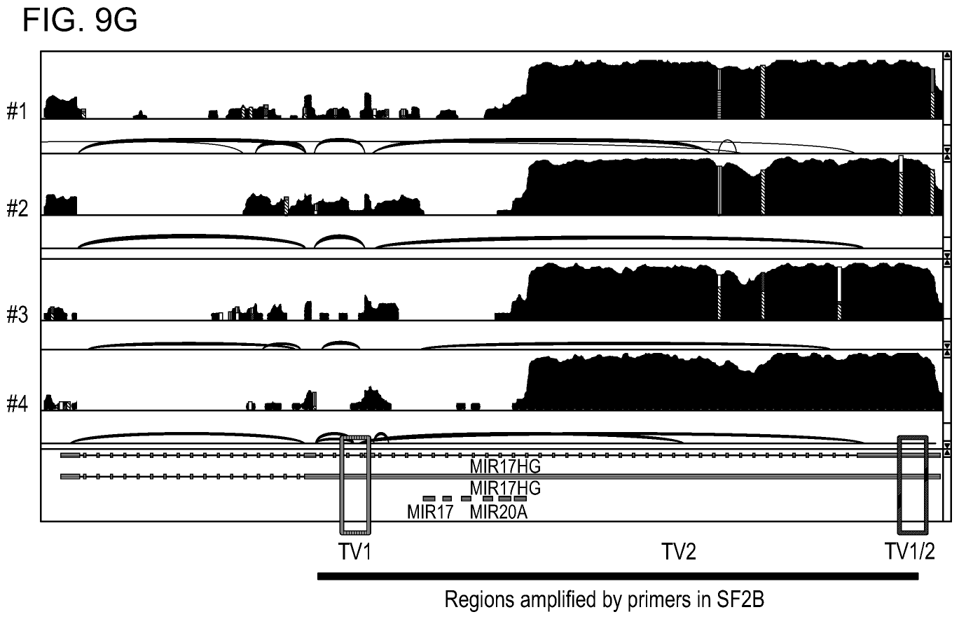

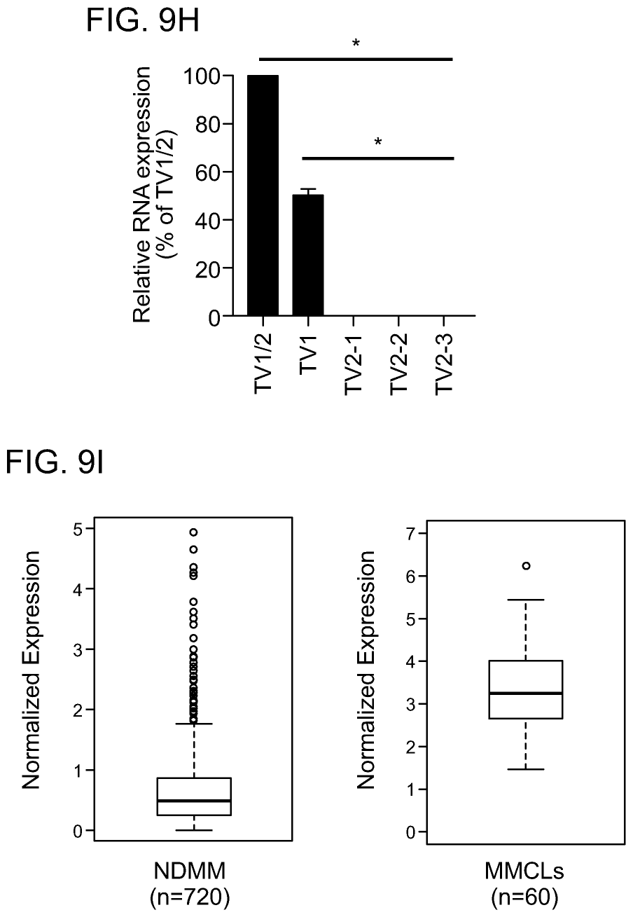

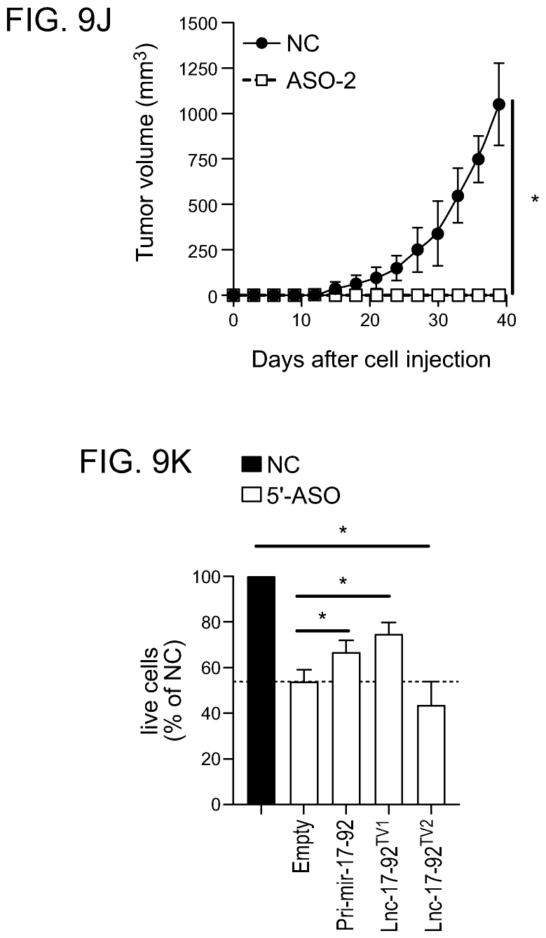

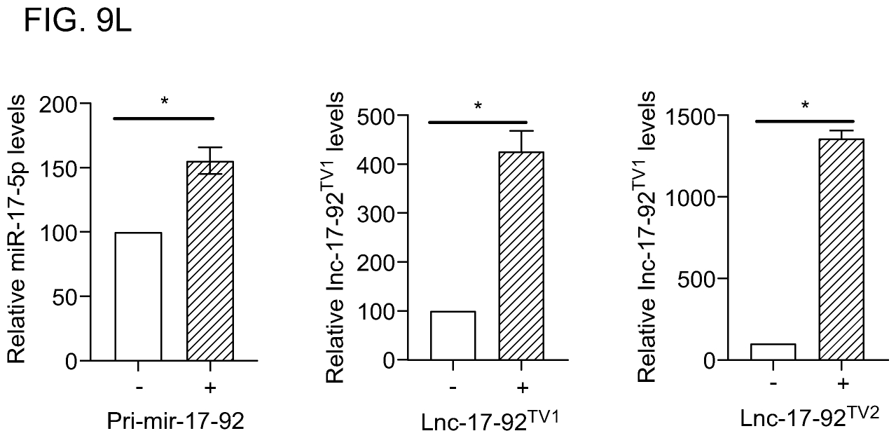

Besides providing a precursor for the microRNA cluster miR-17-92 (MIR17HG^{miR-17-92}; miR-17/-18a/-19a/-20a/-19b/-92a1), MIR17HG also produces as yet poorly, characterized lncRNA transcript Inc-17-92^{TV1} (also known as MIR17HG^{RROL}) (He et al., Nature 435:828-33 (2005); Ota et al., Cancer Res. 64:3087-95 (2004)), as illustrated in FIG. 2A. It was observed that RROL expression was higher during disease progression in 2 independent datasets from MM patients enrolled in the IFM/DFCI clinical trial (NCT01191060) analyzed at diagnosis and/or relapse (see, FIG. 9A-FIG. 9B); and that higher expression of RROL was associated with shorter event-free (EFS) and overall (OS) survival in 3 large cohorts of newly diagnosed MM patients (FIG. 2B). Expression of RROL did not significantly correlate with miR-17-92s in CD138+MM cells from 140 patients (average Spearman r=0.16), suggesting that RROL and miR-17-92 are subject to independent regulatory control and may function in distinct molecular pathways (FIG. 9C).

Supporting a miRNA-independent function of RROL, first, it was observed that an intact anti-proliferative activity of anti-MIR17HG ASOs in presence or absence of ectopic expression of pri-mir-17-92 in two MM cell lines (FIG. 2C and FIG. 9D). The AMO1 and H929 cells were stably transduced with either a lentivector carrying pri-mir-17-92 (pri-miR) or a lentiviral vector carrying GFP as control; and transfected with 2 different ASOs targeting the 5′end (5′-ASO) of MIR17HG pre-RNA or a scrambled control (NC). Effects on cell proliferation were assessed 48 h after transfection. FIG. 9D shows cells infected with a lentiviral vector containing pri-mir-17-92 (Lenti-pri-mir) or a lentiviral vector containing GFP. The results shown are average miRNA expression levels after normalization with RNU44 and ΔΔCt calculations. To solely deplete RROL, ASOs were designed to target MIR17HG at the 5′-end (5′-ASOs), a sequence not included in the ectopic pri-mir-17-92. Second, two DROSHA knockout (DR-KO) MM cell lines (AMO1^{DR-KO} and H929^{DR-KO}) were established, which are unable to produce miR-17-92s (FIG. 9E). The results shown are average miRNA expression levels after normalization with RNU44 and ΔΔCt calculations (Bartel et al., Cell 116:281-97 (2004)). ASO-mediated targeting of MIR17HG pre-RNA solely affects RROL in these cells. A strong anti-proliferative activity in both DR-WT and DR-KO cell systems after RROL depletion was observed, with no significant rescue in DR-KO cells, as assessed after gymnotic treatment with ASO1 (1 μM for AMO1 and 2.5 μM for H929) for 6 days and detected by western blot analysis of DROSHA expression in WT and KO cells (FIG. 2D) or after transfection with 3 different ASOs (−1/-2/-3) (FIG. 9F). Vinculin was used as protein loading control. The percentage of live cells were compared to NC and was analyzed 48 h after transfection by CCK-8 assay. RROL expression was analyzed 48 h after transfection by qRT-PCR. One of three independent experiments are shown.*Indicates p<0.05 after Student t test calculation. In MM cells, both RNA-seq (FIG. 2G) and qRT-PCR (FIG. 2H) indicated preferential expression of RROL TV1, which is the isoform further investigated and hereafter referred to as RROL (also known as Inc-17-92). Using RNA-seq, its expression in CD138+ cells from an additional large cohort of MM patients (MMRF/CoMMpass, n=720) and in MM cell lines (n=60) (FIG. 9I) was confirmed.

Importantly, exposure to ASO1 abrogated the ability of AMO1^{DR-KO} to establish tumors into NOD SCID mice, as detected by tumor growth of AMO1^{DR-KO} with (ASO-1) or without (NC) RROL depletion (FIG. 2E) and prolonged animal survival (FIG. 2F). The easy-to-transfect colorectal cancer cell line HCT-116, which is driven by MIR17HG³⁴, was used to find that ectopic expression of Inc-17-92^{TV1} significantly rescued the anti-proliferative activity of ASOs targeting MIR 17HG pre-RNA more effectively than ectopic Inc-17-92TV2 or pri-mir-17-92 (FIG. 9K and FIG. 9L), transfected with 2.5 μg carrying ectopic pri-mir-17-92, lnc-17-92TV1, Inc-17-92TV2, or GFP (empty). Effects on cell proliferation were assessed 2 days after transfection with 5′-ASO and 3 days after transfection with plasmids. Cells transfected with NC and empty vector were used as reference. Effects on cell proliferation were assessed 48 h after transfection. Ectopic expression of pri-mir-17-92, Inc-17-92TV1, and lnc-17-92TV2, was confirmed by qRT-PCR showing: i) upregulation of miR-17 after ectopic expression of pri-mir-17-92; ii) upregulation of Inc-17-92TV1 after its ectopic expression; iii) upregulation of Inc-17-92TV2 after its ectopic expression. Anti-proliferative activity of anti-MIR17HG ASOs in colorectal cancer cell lines HCT-116 and DLD-1 was also observed, despite these cell lines carrying (−/−) or not (wt) a mutant Dicer conferring hypomorphic phenotype (Cummins et al., Proc. Natl. Acad. Sci. USA 10³:3687-92 (2006)), as illustrated in FIG. 2G. Cells were transfected with either a scramble control (NC) or 2 different ASO (−1 and −2) to obtain RROL depletion. Live cells %, compared to NC, was analyzed 48 h after transfection. *indicates p<0.05, ns indicates p>0.05 after Student t test. These results indicate that RROL is the main mediator of MIR17HG cancer dependency without relationship to miR-17-92.

### Example 4: RROL Interacts with Chromatin to Regulate Gene Expression

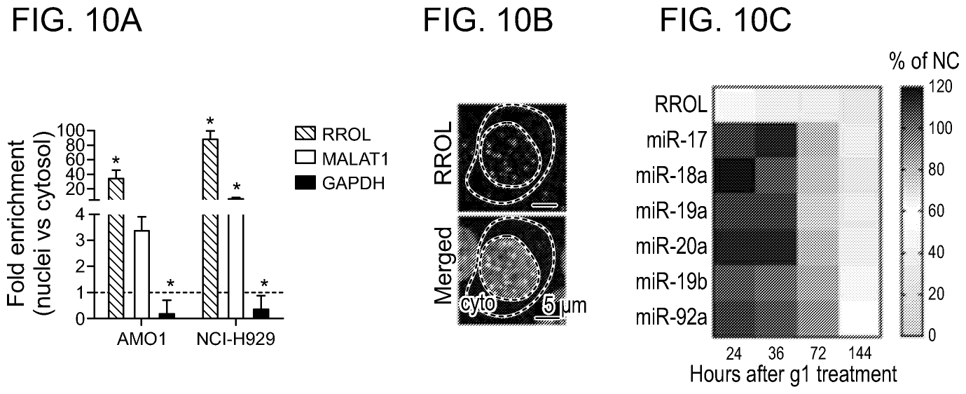

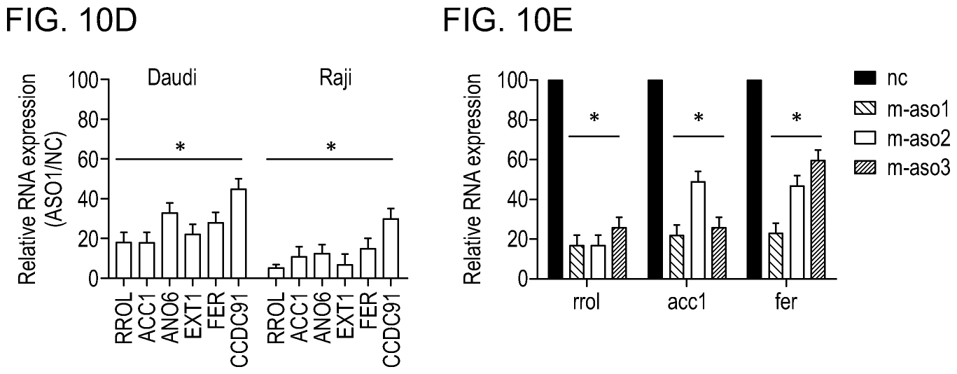

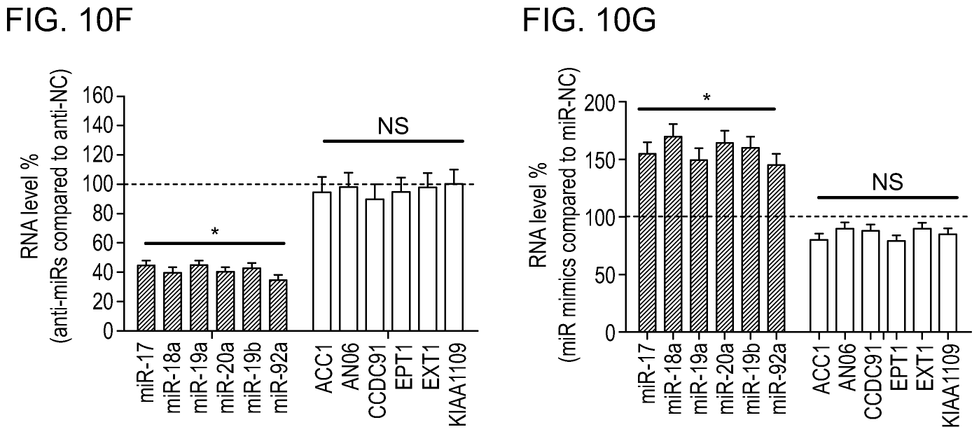

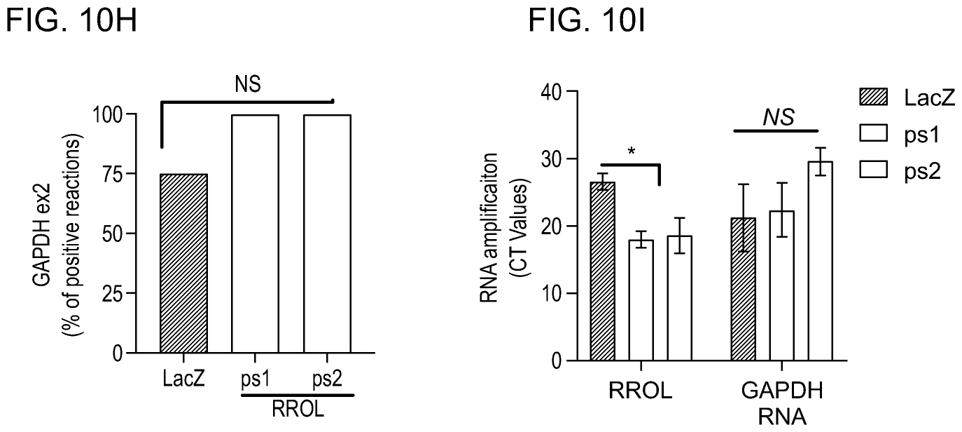

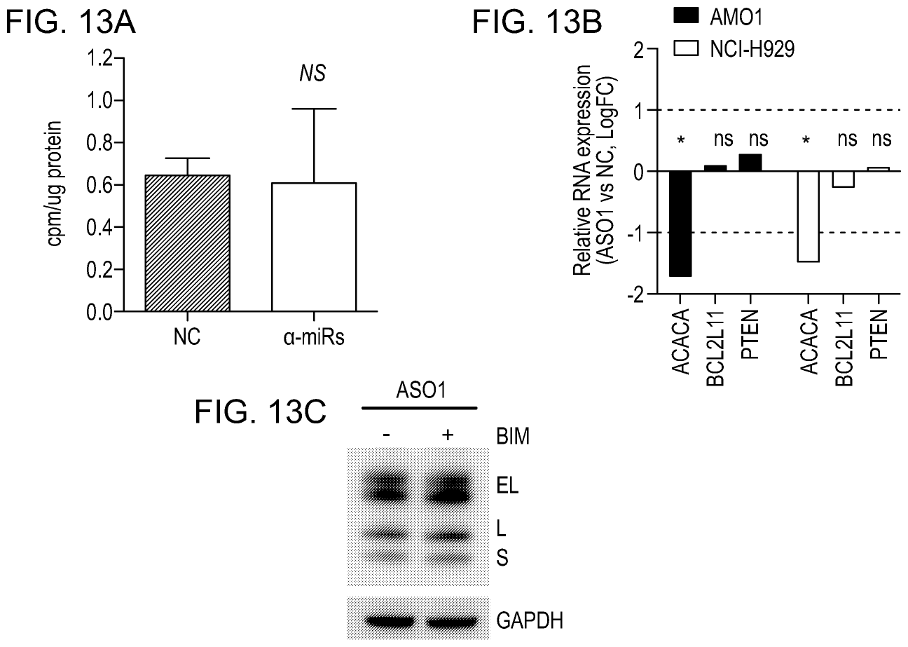

The functional role of lncRNAs depends on their subcellular localization Ulitsky et al., Cell 154:26-46 (2013). qRT-PCR analysis of nuclear and cytosolic compartments, with MALATI and GAPDH mRNA as positive controls, indicated a nuclear enrichment of RROL (FIG. 10A). MALATI and GAPDH were used as internal controls for nuclear- and cytosolic-enriched RNAs, respectively. The results are average fold enrichment of nuclear vs cytosolic fraction after 2-ΔCt calculations. This finding was confirmed by RNA FISH, as illustrated in FIG. 10B. On this basis, next the transcriptional network regulated by RROL in MM was explored. To this end, an ASO-based loss-of function (LOF) study was performed followed by an integrated gene expression analysis in both DR-WT (AMO1 and H929) and DR-KO (AMO1^{DR-KO}) MM cell lines. Through a kinetic analysis of differentially expressed genes (DEGs), performed after early exposure to gymnotic ASO1 to avoid modulation of miR-17-92 in DROSHA WT cells (FIG. 10C) or transfection with ASO2 (FIG. 9K), identified a set of genes rapidly downregulated after RROL depletion in all the cell lines tested. The heatmap shown in FIG. 10C shows average RROL or miRNA expression levels after normalization with GAPDH or RNU44, respectively, and ΔΔCt calculations. RROL was also depleted in DR-WT (AMO1 and H929) and DR-KO (AMOIDR-KO) MM cell lines using early exposure to gymnotic ASO1 to avoid modulation of RROL's canonical targets in DROSHA WT cells (FIG. 13B and FIG. 13C).

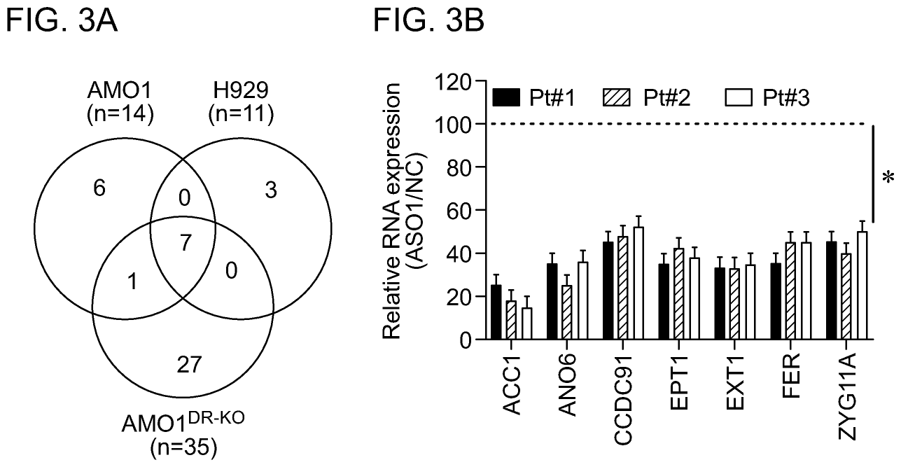

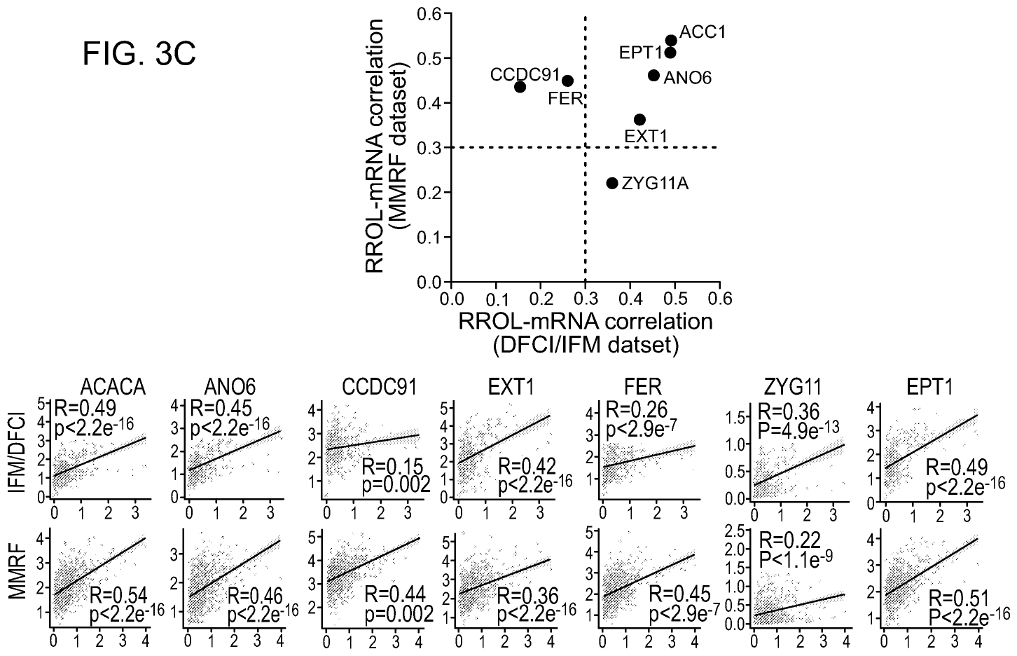

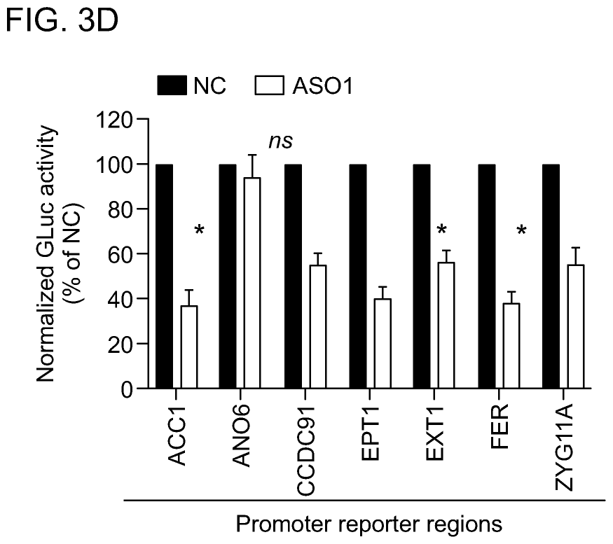

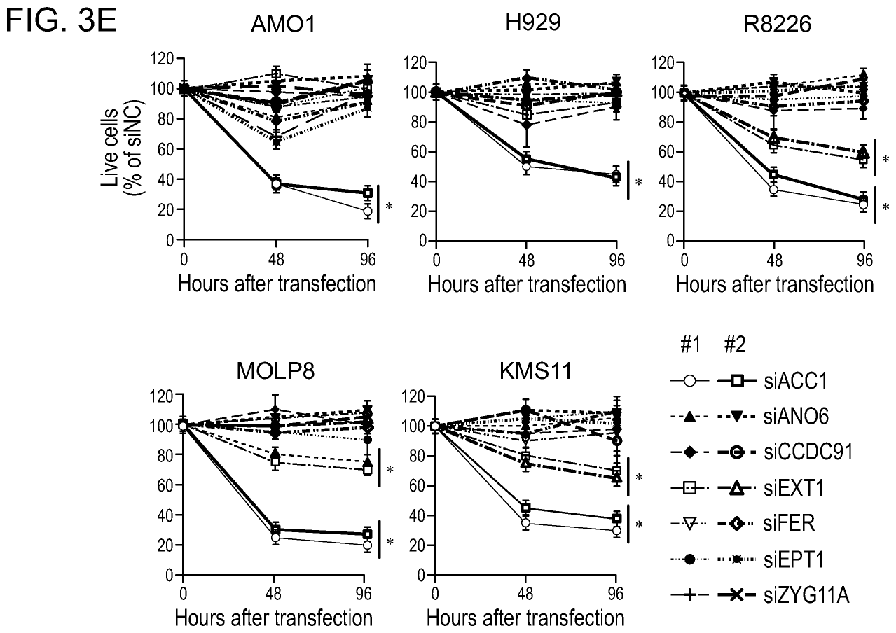

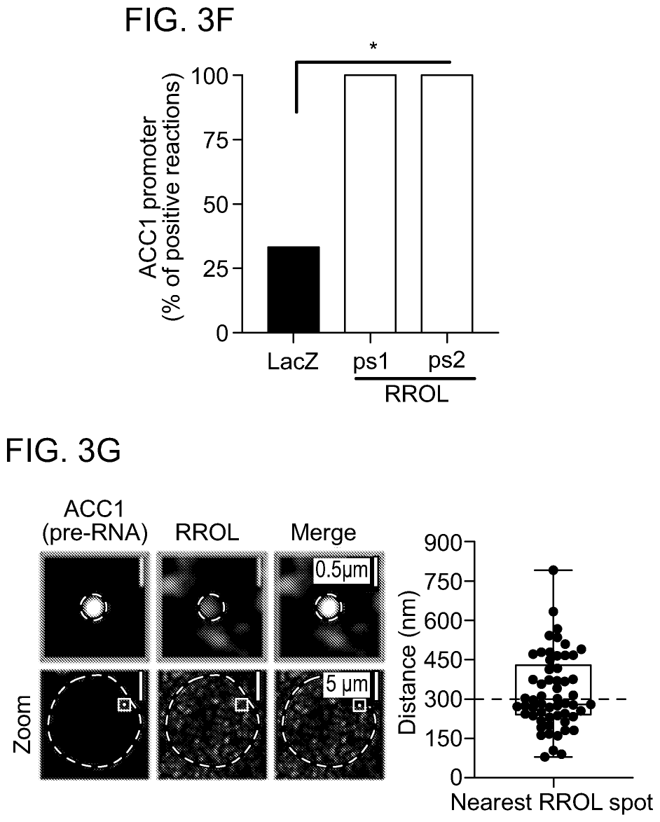

These findings were validated in CD138+ cells from 3 MM patients treated ex vivo with ASO1 (FIG. 3B). The results shown in FIG. 3B are average mRNA expression levels after normalization with GAPDH and ΔΔCt calculations. RNA level in cells exposed to NC (vehicle) were set as an internal reference. The RROL depletion findings were also validated in other cellular models, including the lymphoma cell lines Raji and Daudi (FIG. 10D) and the murine MM cell line 5TGMI depleted of human or murine RROL (FIG. 10E). The results shown in FIG. 10D are the average mRNA expression levels after normalization with GAPDH and ΔΔCt calculations. The results shown in FIG. 10E are the average mRNA expression levels after normalization with murine gapdh and ΔΔCt calculations. Conversely, the expression of these genes was not affected by modulation of individual members of miR-17-92 by synthetic mimics or inhibitors (FIG. 10F-FIG. 10G). The results shown are average miRNA or mRNA expression levels after normalization with RNU44 or GAPDH, respectively, and ΔΔCt calculations. Moreover, significant positive correlation (Spearman r>0.3; p<0.001) between RROL and its target genes in at least 1 out of 2 large RNA-seq MM patient datasets (IFM/DFCI and MMRF/COMMpass) were observed, as illustrated in FIG. 3C; supporting clinical relevance of these regulatory axis. FIG. 3C illustrates a correlation analysis between RROL targets (mRNA) and RROL in CD138+MM patient cells from 2 large RNA-seq cohorts (DFCI/IFM, n=360; MMRF/COMMpass, n=720). It is reported the Spearman r obtained in DFCI/IFM (x axis) and MMRF/COMMpass (y axis) dataset. Dotted red lines indicates r-0.3.

A luciferase reporter assay, performed in 293TDR-KO cells in presence or absence of RROL depletion, demonstrates the regulatory control of RROL over these genes, except ANO6, occurs at the promoter level (FIG. 3D). The reporter vectors were co-transfected into 293T cells with either ASO1 or control ASO. Cells were harvested for luciferase activity assay 48 h after transfection. Results are shown as % of normalized Gluc activity in ASO1 transfected cells compared to control. RNAi-based LOF screen in 5 MM cell lines identified ACC1 (Acetyl-CoA Carboxylase Alpha) as the RROL target gene with most significant impact on MM cell proliferation and survival (FIG. 3E). These results provided the rationale to further explore the role of RROL-ACC1 axis in MM pathobiology. As shown in FIG. 3E, 2 siRNAs were used for each target, plus a scramble siRNA (NC) as control. Cell viability was measured at the indicated time points. It is represented as % of NC transfected cells. Consistently, RROL interaction at the promoter region of the top target ACC1 was confirmed by chromatin isolation by RNA precipitation (ChIRP) assay followed by qRT-PCR analysis (FIG. 3F and FIG. 10H-FIG. 101). Moreover, frequent proximal localization of RROL at the ACC1 gene locus was demonstrated by single molecule dual RNA FISH analysis of RROL and ACC1 pre-mRNA (<300 nm to nearest RROL spot in ˜50% of ACC1 pre-RNA spots analyzed (n=60) (FIG. 3G).

Altogether, without being bound by theory, these data indicate RROL as a chromatin-interacting lncRNA with transcriptional regulatory functions.

### Example 5: RROL Promotes MYC Occupancy at the ACC1 Promoter

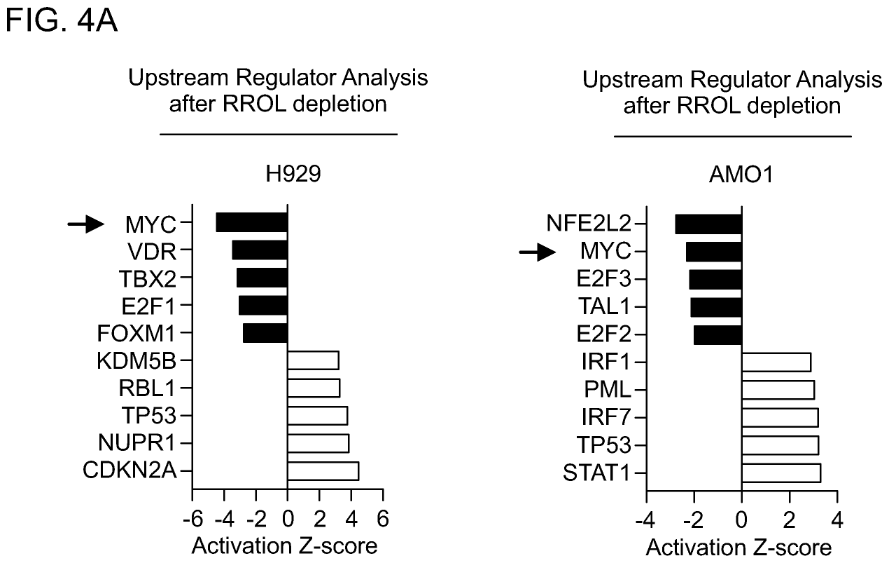

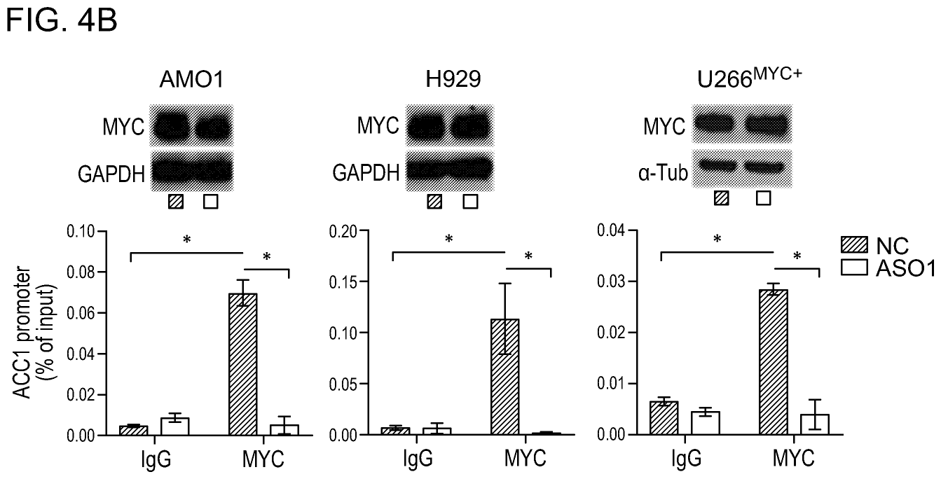

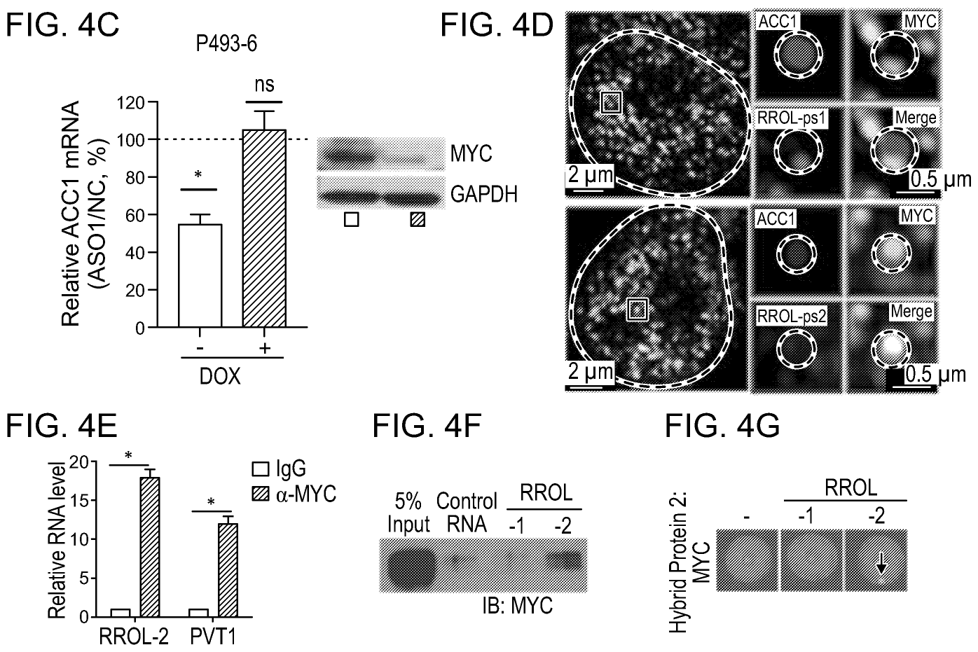

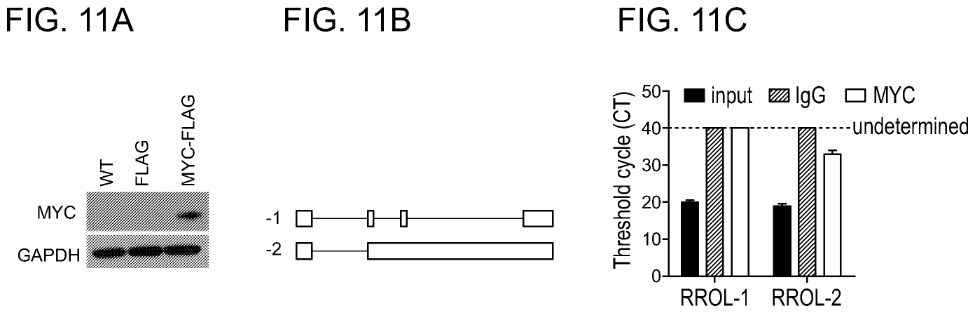

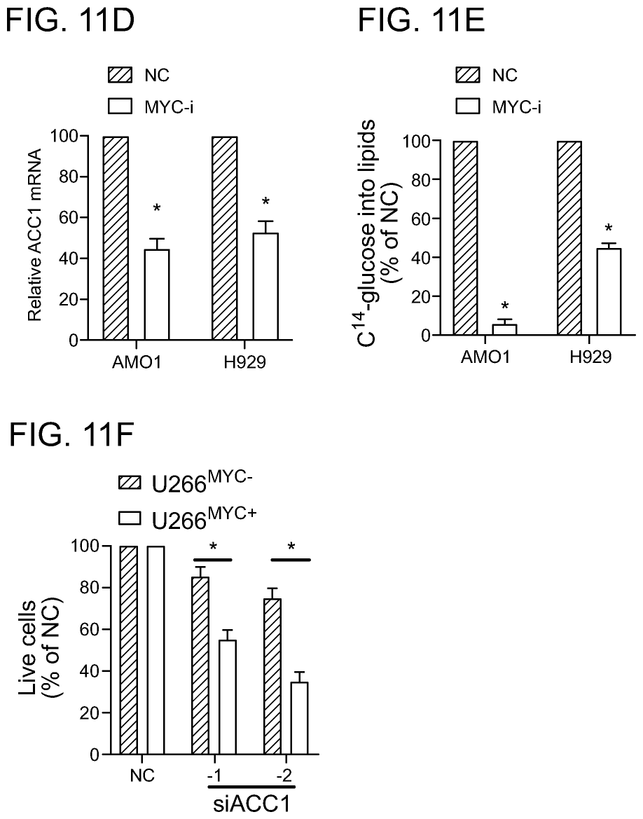

A strong inhibition of the MYC related network upon RROL depletion in both cell lines tested was observed through an upstream regulatory analysis of RROL-related gene expression changes (FIG. 4A). Based on this analysis, it was evaluated if MYC and RROL cooperate to promote ACC1 expression in MM cells. RROL depletion in MM cells indeed abrogated MYC occupancy at the ACC1 promoter, while its expression was not affected (FIG. 4B and FIG. 11A). MYC occupancy at ACC1 promoter was calculated as % of input chromatin. Western blot analysis of MYC from paired samples is shown below each histogram plot. GAPDH or a-tubulin were used as protein loading controls. The expression of ACC1 in the conditional MYC Tet-Off cell line P493-6 (Schuhmacher et al., Curr. Biol. 9:1255-8 (1999)) was reduced only in presence of high MYC levels (FIG. 4C). ACC1 expression levels in cells exposed to NC were set as an internal reference. Moreover, by coupling RNA FISH analysis of RROL and ACC1 pre-RNA with immunofluorescence analysis of MYC protein (FISH/IF), the co-localization of RROL and MYC at the ACC1 gene locus was seen, as illustrated in FIG. 4D. RROL was detected using 2 different probes (RROL-ps1 and RROL-ps2), and a representative image is shown with each RROL probe in FIG. 4D.

To evaluate the existence of a RROL-MYC complex, RNA immunoprecipitation (RIP) assay was performed with MYC antibody, which showed a specific enrichment of RROL isoform 2 (RROL-2) in the MYC-bound RNA, as illustrated in FIG. 4E and FIG. 11B-FIG. 11C. LncRNA PVT1 was used as positive control for the known role as MYC interactor (FIG. 4E). RNA-Protein pull-down (RPPD) experiments confirmed MYC forming a complex with RROL-2 (FIG. 4F and FIG. 11D). Furthermore, the RNA yeast-3-hybrid (Y3H) assay was adapted to confirm the RROL-MYC interaction in an in vivo cellular model (Hook et al., RNA 11:227-33 (2005)). In this assay, a direct RROL-MYC interaction activates a reporter gene allowing for yeast colony growth; and, as shown in FIG. 4G, we detected yeast colony growth in the presence of RROL-2 as hybrid RNA.

These data appear to demonstrate that RROL forms an RNA-protein complex with the transcription factor MYC to promote its chromatin occupancy and transcriptional activity at the ACC1 promoter.

### Example 6: RROL mediates the assembly of a MYC-WDR82 transcriptional complex, leading to transcriptional and epigenetic activation of ACC1

The targeting of MIR17HG primarily kills c-MYC positive (MYC+) tumor cells, including in MM. Intriguingly, MYC is known to reactivate ACC1 (also known as ACACA) expression and de novo lipogenesis in tumor cells, with MYC+ tumor cells becoming addicted to this metabolic pathway, which are validated in MM cells herein (FIGS. 11D-FIG. 11F). FIG. 11D shows ACC1 mRNA following treatment with MYC inhibitor 10058-F4 in MM cell lines AMO1 and H929. Raw Ct values were normalized to GAPDH mRNA and expressed as ΔΔCt values calculated using the comparative cross threshold method. ACC1 expression levels in cells treated with DMSO (NC) were set as an internal reference. Therefore, the functional interplay between MYC and MIR 17HG was investigated.

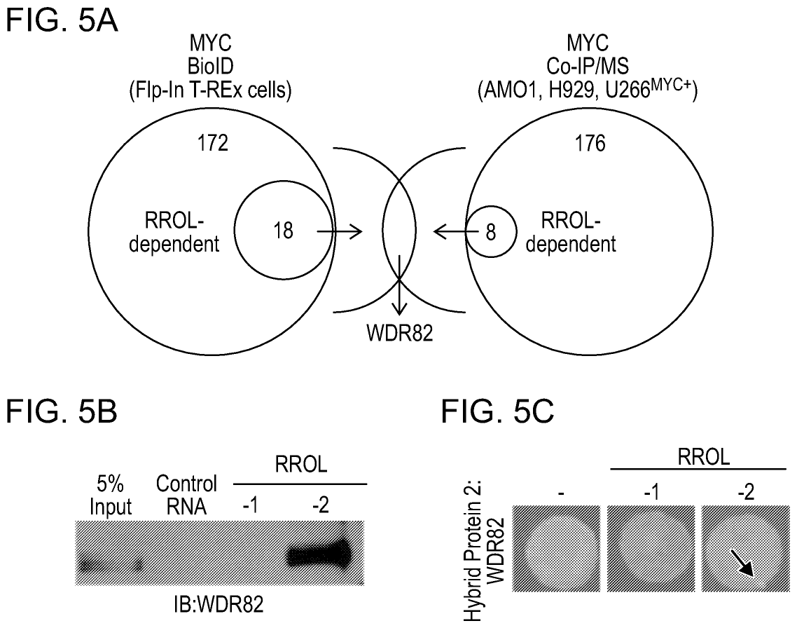

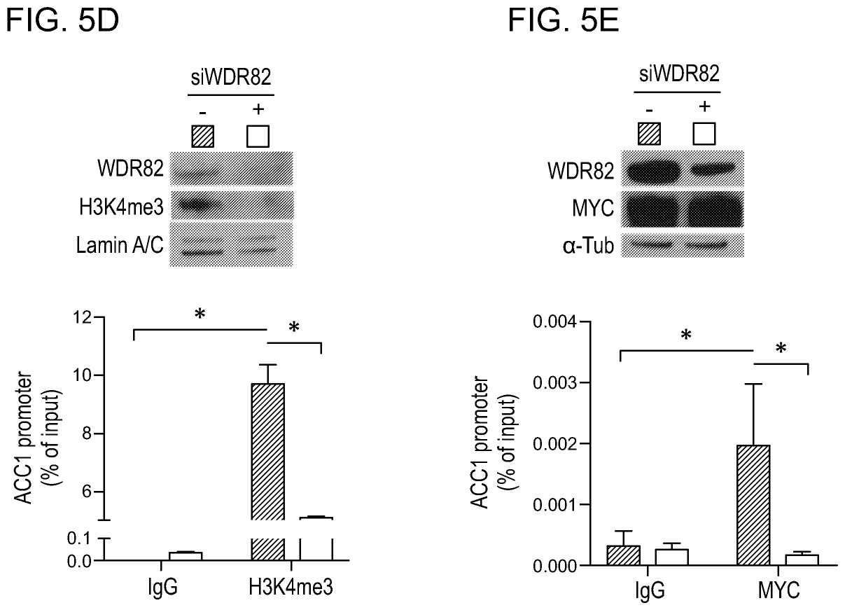

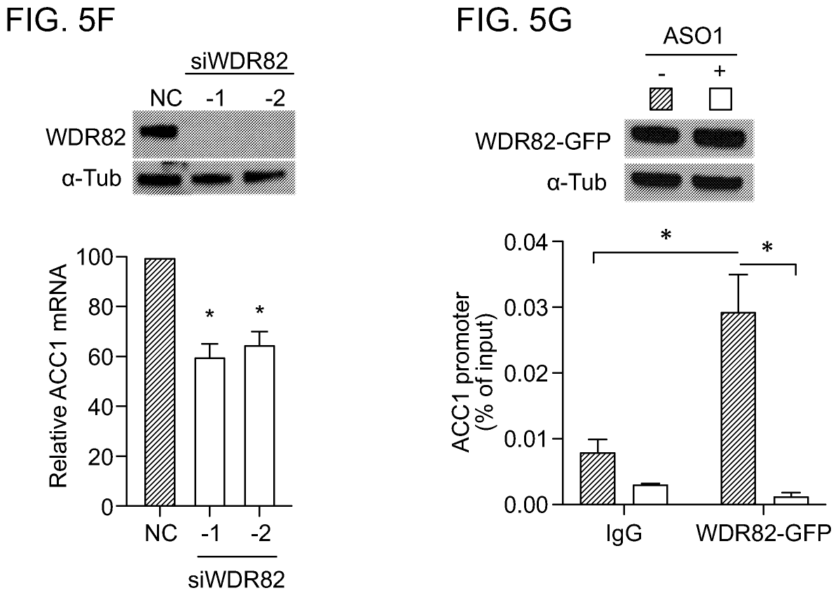

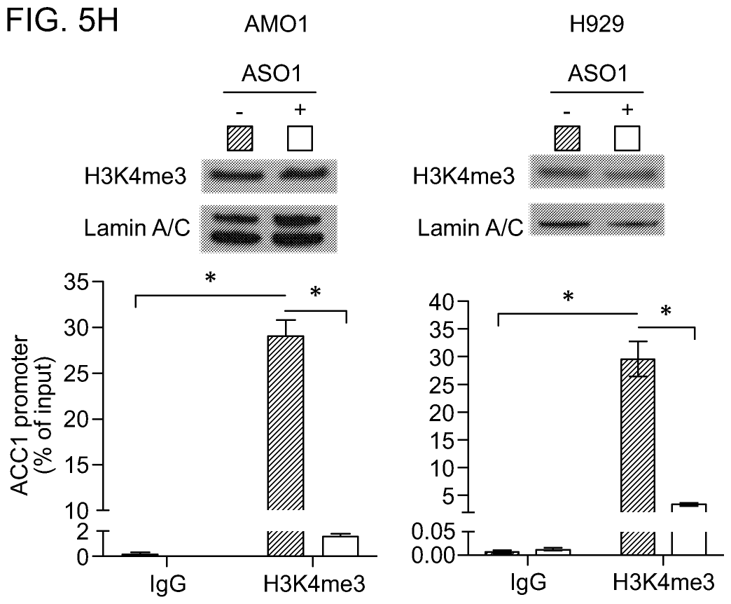

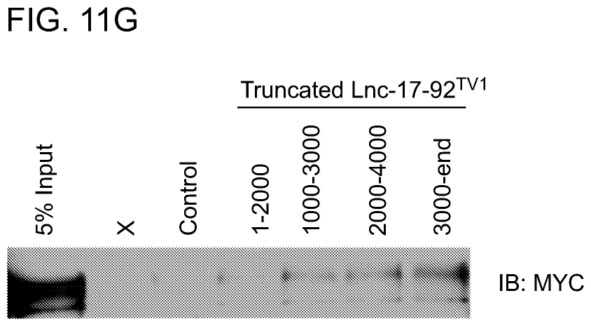

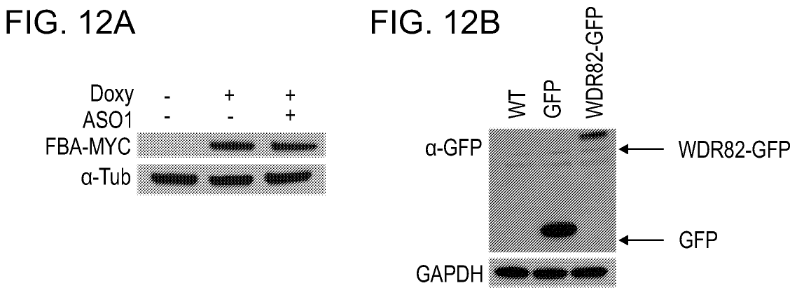

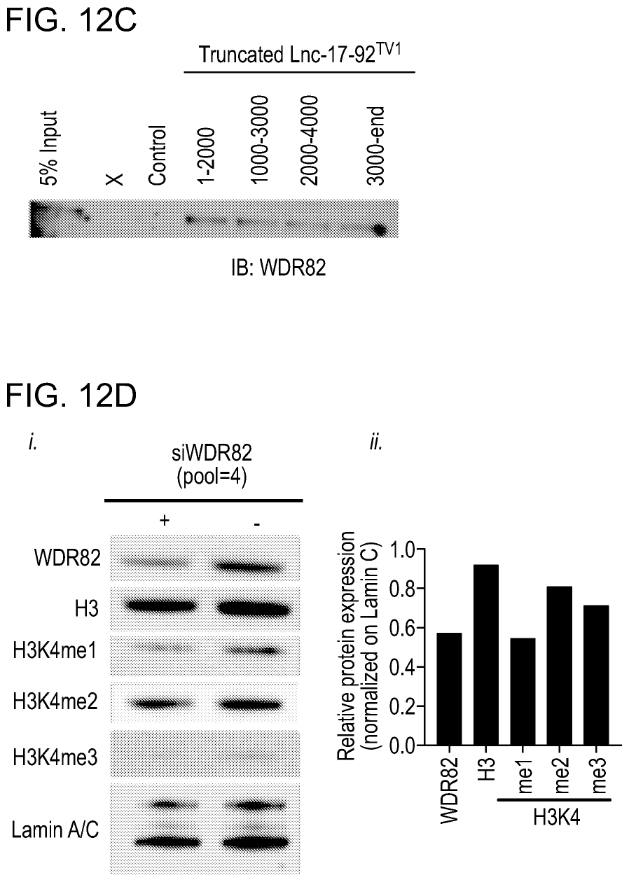

MYC activity has been shown to be modulated through the interaction with transcriptional and epigenetic co-regulators (Gouw et al., Cell Metab. 30:556-572 (2019)). To determine if RROL affects these protein-protein interactions, the results of proximity-dependent biotin identification (BioID) analysis were integrated with co-immunoprecipitation assay followed by mass-spectrometry analysis (Co-IP/MS) in 3 MM cell lines (AMO1, H929 and U266^{MYC+}), in the presence and in the absence of RROL depletion. This integrated analysis highlighted WDR82 as a very high-confidence RROL-dependent MYC interactor (FIG. 5A and FIG. 12A, and Table 5-Table 8); A direct RNA-protein interaction between RROL and WDR82 was further confirmed by both RPPD (FIG. 5B) and RNA Y3H (FIG. 5C) assays. Black arrows in FIG. 5C indicate yeast colony growth. An analysis using truncated versions of Inc-17-92TV1 further indicated the 3′-end regions, which do not include miR-17-92, as particularly relevant for the interaction with MYC in MM cells (FIG. 11G).

All 172 MYC interactors in vivo are listed in Table 5. All 18 RROL-dependent in vivo MYC interactors identified from the BioID assay are listed in

Table 6. All 176 MYC interactors in MM cells (as identified in more than 2 cell lines) are listed in Table 7. All 8 RROL-dependent MYC interactors in MM cells (as identified in more than 2 cell lines) are listed in Table 8.

TABLE 5
MYC Interactors in vivo (n = 172)

| | P | | P | | P |
|---|---|---|---|---|---|
| Gene | value | Gene | value | Gene | value |
| AARS2 | 0.000 | TOR1AIP1 | 0.012 | ASB6 | 0.028 |
| EDC4 | 0.000 | HCFC1 | 0.012 | SNW1 | 0.029 |
| KDM3B | 0.000 | AP3B1 | 0.012 | CDK12 | 0.03 |
| DMAP1 | 0.000 | RPRD1B | 0.012 | WDR6 | 0.031 |
| IPO4 | 0.000 | CPSF4 | 0.012 | CANX | 0.031 |
| RUVBL2 | 0.000 | ARF5 | 0.013 | ANKFY1 | 0.032 |
| NCOR1 | 0.000 | CHD1 | 0.014 | TOX4 | 0.032 |
| WDR82 | 0.001 | FAM96B | 0.014 | GIPC1 | 0.033 |
| SAE1 | 0.001 | U2SURP | 0.014 | SMAD5 | 0.033 |
| ATP1A1 | 0.001 | GEMIN5 | 0.014 | CDCA2 | 0.033 |
| FIP1L1 | 0.001 | TAF5L | 0.015 | PSME3 | 0.034 |
| TUBB2B | 0.002 | TPR | 0.015 | SALL2 | 0.034 |
| PDCD6 | 0.002 | ERLIN2 | 0.015 | ATP2B4 | 0.035 |
| GTF2E2 | 0.002 | STAT3 | 0.016 | MAGED2 | 0.035 |
| RPL14 | 0.002 | ARID3B | 0.016 | USP10 | 0.035 |
| SAP30BP | 0.003 | SAR1B | 0.016 | CHD8 | 0.035 |
| SRPRB | 0.003 | MAD2L1 | 0.017 | ALAS1 | 0.035 |
| SAFB | 0.003 | NCOR2 | 0.017 | API5 | 0.035 |
| RSU1 | 0.003 | PBRM1 | 0.017 | R0214 | 0.035 |
| DHFR | 0.003 | DIDO1 | 0.017 | EMD | 0.036 |
| SMUG1 | 0.004 | IRF2BP2 | 0.017 | CBLL1 | 0.036 |
| TUBB4A | 0.004 | RACGAP1 | 0.017 | BIN3 | 0.036 |
| RUVBL1 | 0.005 | ACTL6A | 0.017 | MAP4 | 0.036 |
| MAP7D1 | 0.005 | EEF1A2 | 0.018 | SETDB1 | 0.036 |
| LSM2 | 0.005 | CIZ1 | 0.018 | HBA1 | 0.036 |
| SF1 | 0.005 | EIF2B2 | 0.018 | IPO9 | 0.037 |
| DKFZp686L22104 | 0.006 | Z0318 | 0.018 | SEC22B | 0.037 |
| NOB1 | 0.006 | CBWD1 | 0.019 | OSBP | 0.037 |
| PRRC2B | 0.006 | ITPA | 0.019 | MDC1 | 0.037 |
| ECD | 0.006 | CSNK2A2 | 0.019 | CEP85 | 0.038 |
| RPRD2 | 0.006 | TUBB | 0.02 | SAR1A | 0.038 |
| ATXN10 | 0.006 | IQGAP2 | 0.02 | TAF6 | 0.038 |
| TOR1AIP1 | 0.006 | TRNT1 | 0.02 | PPM1G | 0.038 |
| TAF6L | 0.007 | NELFA | 0.022 | ZBTB33 | 0.039 |
| TK1 | 0.007 | GAPVD1 | 0.022 | HSPA14 | 0.039 |
| PTRH2 | 0.007 | EIF2S2 | 0.022 | RIF1 | 0.039 |
| SCML2 | 0.007 | RTKN | 0.023 | SRP72 | 0.039 |
| TUBA1C | 0.007 | LRRC47 | 0.023 | SNRNP40 | 0.04 |
| PSMD11 | 0.008 | XPO5 | 0.023 | HIST1H1C | 0.041 |
| TRIM4 | 0.008 | PSMB6 | 0.023 | LIMA1 | 0.041 |
| SF3A1 | 0.008 | TUBB4B | 0.024 | AP3M1 | 0.041 |
| EP400 | 0.008 | CTPS2 | 0.024 | TTLL12 | 0.042 |
| NUBP2 | 0.008 | EIF5 | 0.024 | EMC4 | 0.043 |
| GIGYF2 | 0.008 | JUN | 0.024 | CHORDC1 | 0.044 |
| ARID1A | 0.008 | MGA | 0.025 | IPO8 | 0.044 |
| DVL2 | 0.009 | COG8 | 0.025 | GATAD2A | 0.045 |
| GTF2F2 | 0.01 | HAT1 | 0.026 | ZC3H14 | 0.045 |
| TTC37 | 0.01 | RPL37A | 0.026 | AP2B1 | 0.047 |
| STAU1 | 0.01 | RING1 | 0.026 | C6orf120 | 0.047 |
| PIGU | 0.01 | NUFIP2 | 0.027 | FANCI | 0.047 |
| CLPTM1 | 0.011 | DYNC1LI1 | 0.027 | POLR2B | 0.047 |
| EIF4E | 0.011 | TCAF1 | 0.027 | KTI12 | 0.047 |
| TRRAP | 0.011 | WDR5 | 0.028 | GON4L | 0.048 |
| XRCC6 | 0.011 | HAUS8 | 0.028 | EIF3H | 0.048 |
| SCAF4 | 0.011 | USP33 | 0.028 | NUP62 | 0.049 |
| CRTC3 | 0.011 | GPATCH8 | 0.028 | VBP1 | 0.049 |
| ITPK1 | 0.012 | PCGF6 | 0.028 | RPL30 | 0.049 |
| I02 | 0.012 | | | | |

TABLE 6
RROL-dependent in vivo MYC interactions
from BioID assay (n = 18)

| Gene | P value | Gene | P value | Gene | P value |
|---|---|---|---|---|---|
| AARS2 | 0.000 | GIGYF2 | 0.003 | CRTC3 | 0.006 |
| KDM3B | 0.000 | AP3B1 | 0.004 | EEF1A2 | 0.007 |
| PTRH2 | 0.001 | DMAP1 | 0.005 | SAE1 | 0.007 |
| SNW1 | 0.001 | WDR82 | 0.006 | RUVBL2 | 0.007 |
| PRRC2B | 0.001 | EIF4E | 0.006 | FAM96B | 0.008 |
| EDC4 | 0.002 | RPRD2 | 0.006 | ARID3B | 0.009 |

TABLE 7
MYC interactors in MM cells (>2 cell lines; n = 176)

| Names | Total | Elements |
|---|---|---|
| AMO1 | 7 | MAX, CDSN, NELFCD, WDR82, CAPNS1, RPL34, RNASEH2A |
| H929 | | |
| U266^{MYC+} | | |
| AMO1 | 137 | RPS17, NCBP2-AS2, HSPB1, PMPCA, AIFM1, MRPL4, NAXE, |
| H929 | | COX6C, LONP1, PTCD3, FAHD1, PRDX6, TUBAL3, GRPEL1, |
| | | PDIA3, MRPL19, PCMT1, SRI, MYO1C, RPLP1, GMPPA, SF3B6, |
| | | GDI1, ARHGEF2, EFTUD2, COX7A2, ACTR1A, ISOC2, CBR4, |
| | | HSPA8, GET4, DHX15, ARPC3, RPS8, OSGEP, RSU1, SSR3, |
| | | PPP3CC, IFI16, OXCT1, LSM3, MOGS, SNRPE, NDUFA13, FAU, |
| | | PAFAH1B3, QPCTL, SELENOF, HIST1H2BB, PCNA, 43710, |
| | | EIF2B2, NAP1L4, ATP6V1A, PSIP1, MYC, RFC4, GNB2, RPS24, |
| | | VCP, PSMA5, ACTN2, PSMC1, STMN1, Ighg1, GAR1, NDUFB3, |
| | | CNOT9, PARP1, FMNL1, BAG6, PSMA2, PSMA4, BRI3BP, |
| | | WDR61, PPIH, TTLL12, LSP1, NSF, NAMPT, BLMH, PRSS2, |
| | | RPS10, IGKC, COPE, UQCC2, EIF4A1, RPL15, MRPL39, DDX3Y, |
| | | HSPA14, PABPC4, ARFIP1, DKFZp686J1372, HNRNPAB, AIMP1, |
| | | SYNCRIP, MRPS23, SNRPGP15, MTAP, MSI2, TMEM109, |
| | | PAFAH1B2, SORD, MCM4, RPL9, PDHX, GNG7, RPA3, ISOC1, |
| | | SEC11A, CAT, CANX, SRM, NDUFA9, ARF3, NUDT5, PSMB3, |
| | | HDGF, SDF2L1, MPRIP, TRMT10C, PSME3, NDUFS3, ECI1, |
| | | PAM16, USP39, RPL38, RPL14, IDH3B, EIF3G, HNRNPA1L2, |
| | | CCT6A, FKBP2, NDUFA5, NCLN, DNAJC19 |
| AMO1 | 9 | LAMTOR3, ARPC2, FAM207A, ASL, SDC1, BAG2, FLOT1, MIF, |
| U266^{MYC+} | | MAGED2 |
| H929 | 23 | MRPS35, THRAP3, YARS2, PPIL1, C19orf25, FAM98B, |
| U266^{MYC+} | | ALDH1L2, SEC13, PSMC4, FLNB, DKC1, AUH, TPM4, GMPPB, |
| | | ACTR1B, RAD50, PSMD4, HLA-B, HNRNPA3, CDKN2C, |
| | | POLR2E, DDX41, TOP1 |

TABLE 8
RROL-dependent MYC interactors in MM cells
(>2 cell lines; n = 8)

| Names | Total | Elements |
|---|---|---|
| AMO1 H929 U266^{MYC+} | 1 | WDR82 |
| AMO1 H929 | 5 | RNASEH2A, COX6C, |
| | | ARFIP1, IDH3B, RPA3 |
| H929 U266^{MYC+} | 2 | C19orf25, RAD50 |

WDR82 is a regulatory component of the SET1 methyltransferase complex catalyzing the histone H3 ‘Lys-4’ trimethylation (H3K4me3) at the transcription start sites of active loci (Lee and Skalnik, Mol. Cell. Biol. 28:609-18 (2008)), a sine qua non condition for MYC binding to chromatin and transactivation Amente et al., Am. J. Cancer Res. 1:413-418 (2011). Consistently, depletion of WDR82 resulted in a global reduction of H3K4me3 (FIG. 5D and FIG. 12D) and a reduced occupancy of MYC at ACC1 promoter (FIG. 5E), resulting in decreased ACC1 expression (FIG. 5F) in MM cells. FIG. 5D shows a western blot analysis of WDR82 and H3K4me3 (represented as % of input chromatin). Lamin A/C was used as protein loading controls (nuclear lysates). FIG. 5E shows a western blot analysis of WDR82 and MYC from paired samples (represented as % of input chromatin). α-tubulin was used as protein loading controls. Data in FIG. 5F are shown as raw Ct values that were normalized to GAPDH mRNA and expressed as ΔΔCt values calculated using the comparative cross threshold method. ACC1 expression levels in ells transfected with NC were set as an internal reference.

FIG. 5G illustrates a ChIP-qPCR analysis of WDR82-GFP occupancy at ACC1 promoter in AMO1 exposed for 24 h to gymnotic ASO1 as a western blot analysis of WDR82-GFP from paired samples (represented as % of input chromatin). α-tubulin was used as protein loading controls. Furthermore, using MM cells expressing an ectopic WDR82-GFP fusion protein (FIG. 12B), it was demonstrated that RROL expression is essential for WDR82 occupancy at the ACC1 promoter (FIG. 5H), without globally impacting the H3K4 methylation status (FIG. 51). FIG. 5H illustrates a ChIP-qPCR analysis of H3K4me3 occupancy at ACC1 promoter in AMO1 and H929 exposed for 24 h to gymnotic ASO1 as a shown a western blot analysis of H3K4me3 from paired samples (represented as % of input chromatin). Lamin A/C was used as protein loading controls (nuclear lysates). * p<0.05, Student t test. Additionally, RROL depletion resulted in reduced levels of H3K4me3 at the ACC1 promoter site, without detectable impact on the total H3K4me3 (FIG. 51).

Showing a role of RROL as a chromatin scaffold mediating the assembly of MYC-WDR82 multiprotein transcriptional complexes, to control the expression of ACC1.

### Example 7: The RROL/MYC-ACC1 Axis Regulates De Novo Lipogenesis

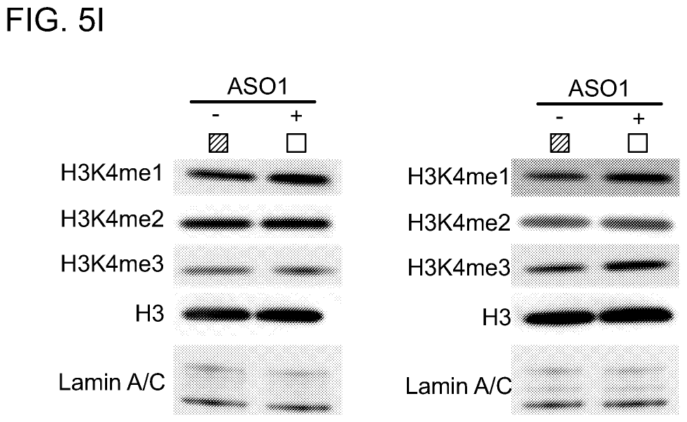

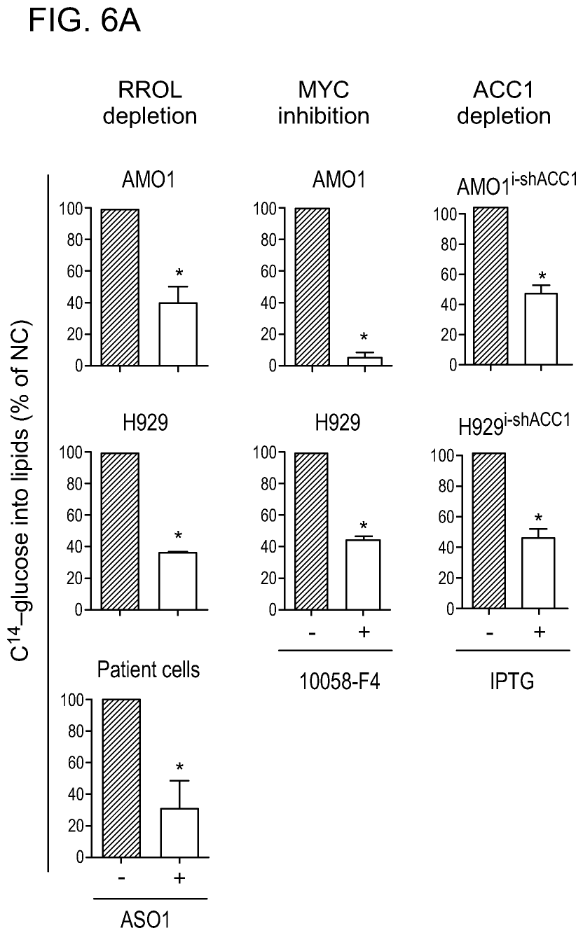

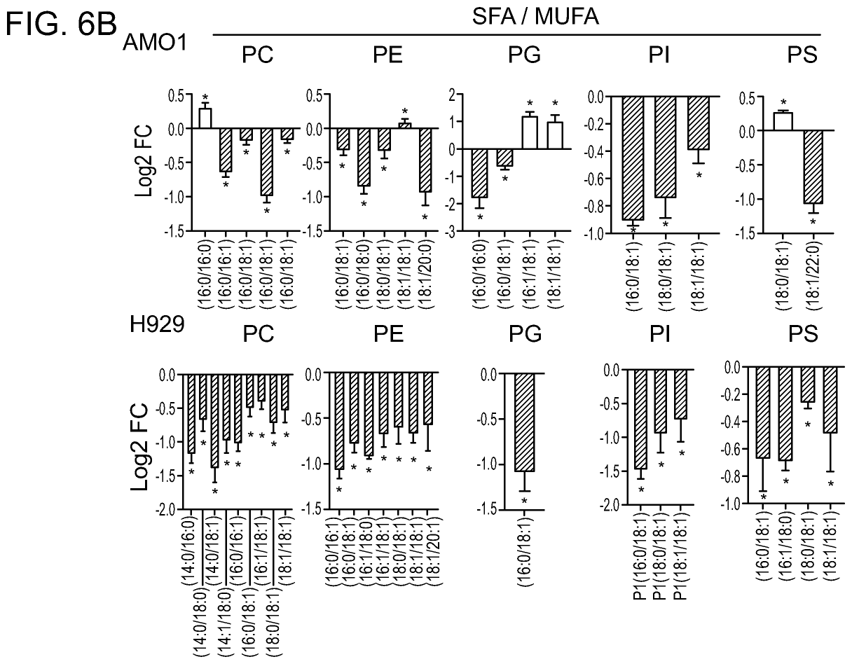

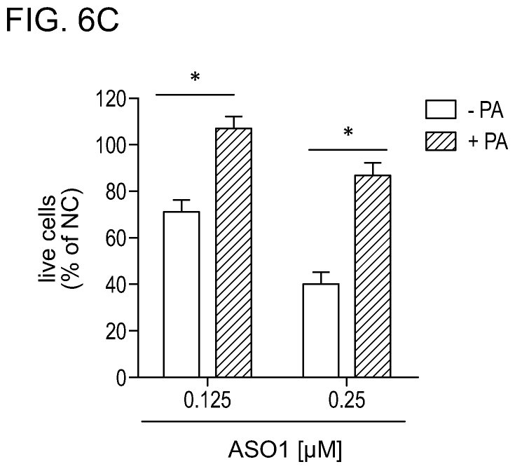

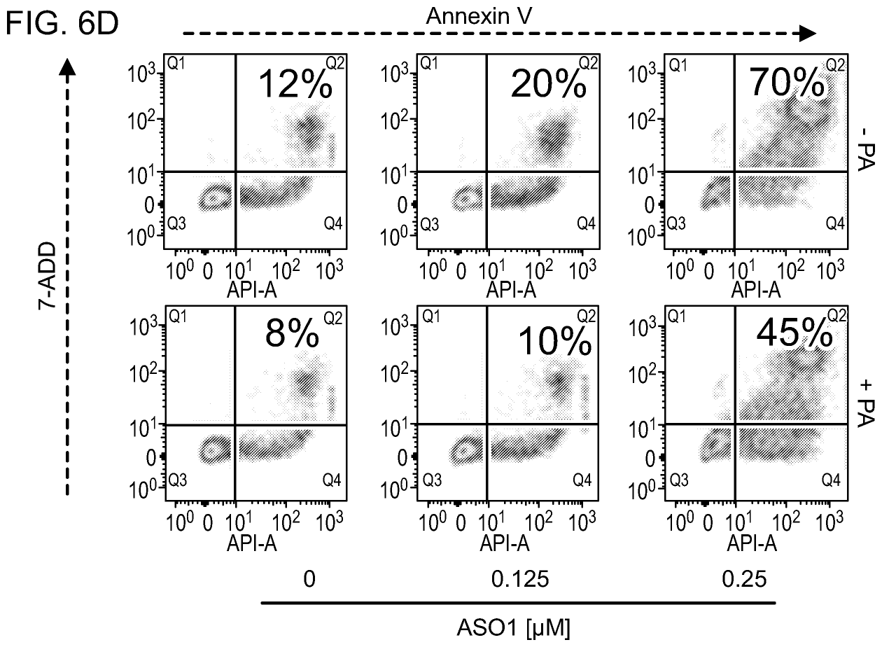

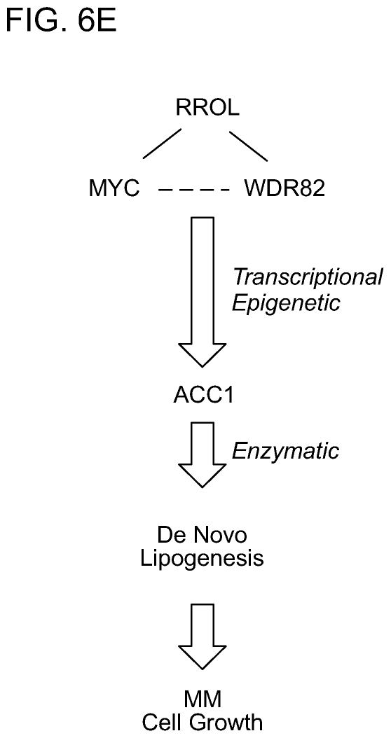

ACC1 catalyzes the carboxylation of acetyl-CoA into malonyl-CoA, the rate limiting step during de novo lipogenesis (DNL) (Beloribi-Djefaflia et al., Oncogenesis 5: e189-10 (2016)), a metabolic pathway aberrantly activated in cancer cells (Röhrig and Schulze., Nat. Rev. Cancer 16:732-749 (2016)). Here, RROL depletion, similarly to either MYC or ACC1 inhibition, significantly reduced the incorporation of C¹⁴-radiolabeled glucose into the lipid pool—indicative of a reduced DNL (Zadra et al., Proc. Natl. Acad. Sci. USA 116:631-640 (2019))—both in MM cell lines and CD138+MM patient cells (FIG. 6A). RROL was depleted using ASO1, MYC was inhibited using small molecule 10058-F4, and ACC1 was depleted using IPTG-inducible shRNAs. Results are expressed as percentage of negative controls (NC ASO, DMSO, and uninduced, respectively). This inhibition was not observed after transfection of MM cells with synthetic inhibitors of miR-17-92s (FIG. 13A), which illustrates the incorporation of C14-glucose into lipids, 48 h after transfection of AMO1 with miR-17-92 anti-miRs (25 nM each). Results are expressed as percentage of uninduced cells. NS indicates p>0.05 after Student t test. Liquid Chromatography-Mass Spectrometry (LC-MS) based lipid profiling after RROL inhibition in MM cells confirmed depletion of several saturated (SFA) and monounsaturated (MUFA) phospholipid species (FIG. 6B) produced via DNL. The lipid profiling analysis showing modulation of major membrane phospholipid classes [phosphatidylglycerol (PG), phosphatidylethanolamine (PE), phosphatidylserine (PS), phosphatidylcholin (PC) and phosphatidylinositol (PI)], with saturated (SFA) or monounsaturated (MUFA) acyl chains, after treatment of AMO1 and H929 with ASO1 is shown in FIG. 6B. (Rysman et al., Cancer Res. 70:8117-26 (2010); Zaidi et al., Prog. Lipid Res. 52:585-9 (2013)). Moreover, addition of the main downstream product of ACC1 activity, palmitate (PA), significantly rescued the anti-proliferative (FIG. 6C, CCK-8 proliferation assay) and pro-apoptotic (FIG. 6D) effects of RROL depletion in MM cells, confirming role of DNL in the tumor promoting activity of RROL in MM and justifying the name of RNA Regulator of Lipogenesis, as illustrated in the proposed model of FIG. 6E. FIG. 6D shows an annexin V/7-AAD flow cytometry assay in AMO1 exposed for 6 days to ASO1 at indicated concentrations, with supplementation of either BSA (control) or BSA-PA (20 μM).

### Example 8: Therapeutic Inhibitors of RROL Exert Potent Anti-Tumor Activity In Vitro and In Vivo in Animal Models of Human MM

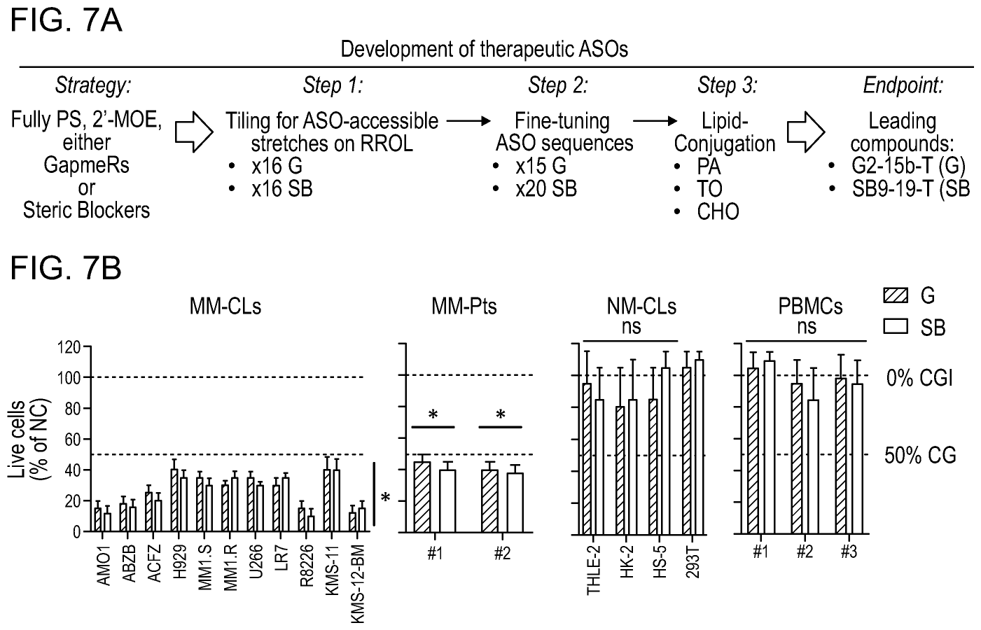

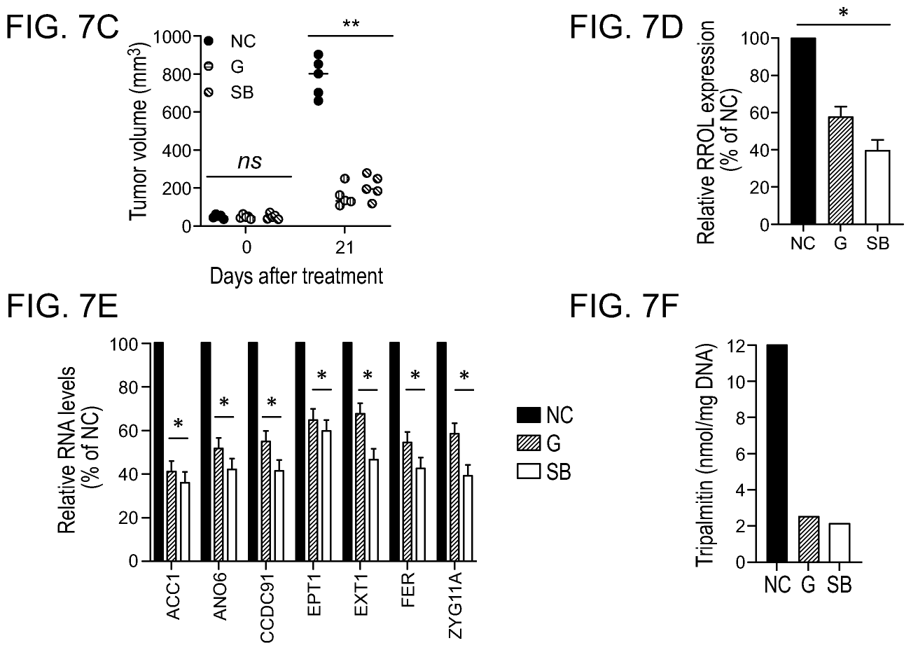

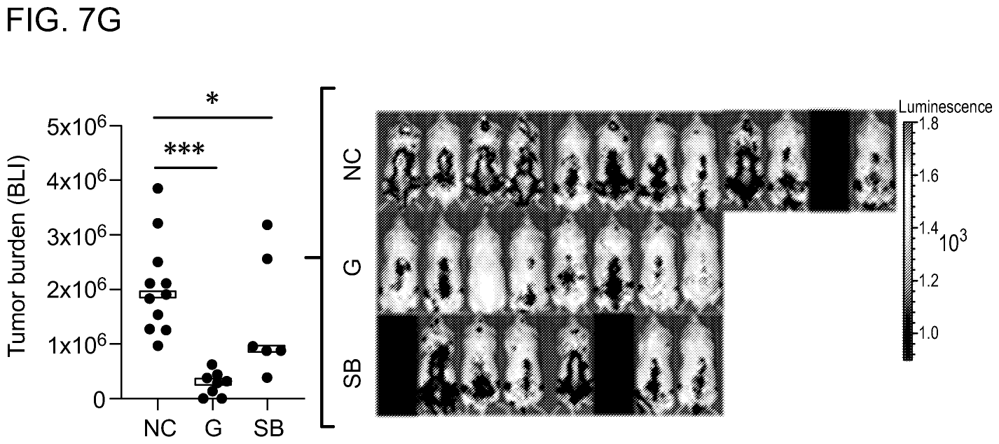

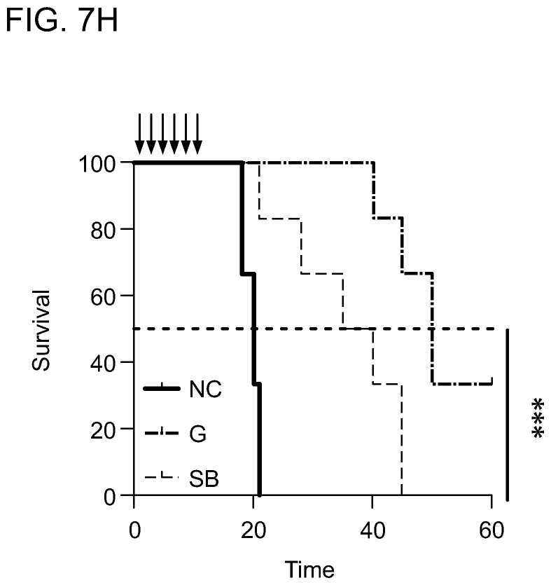

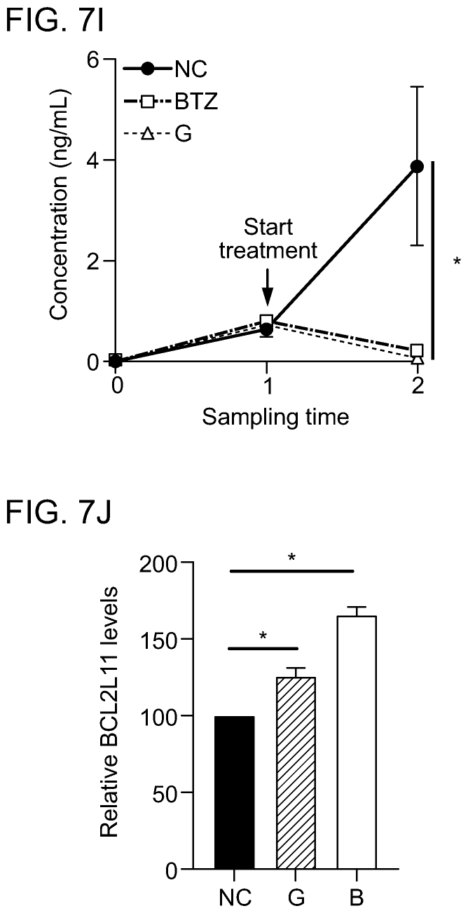

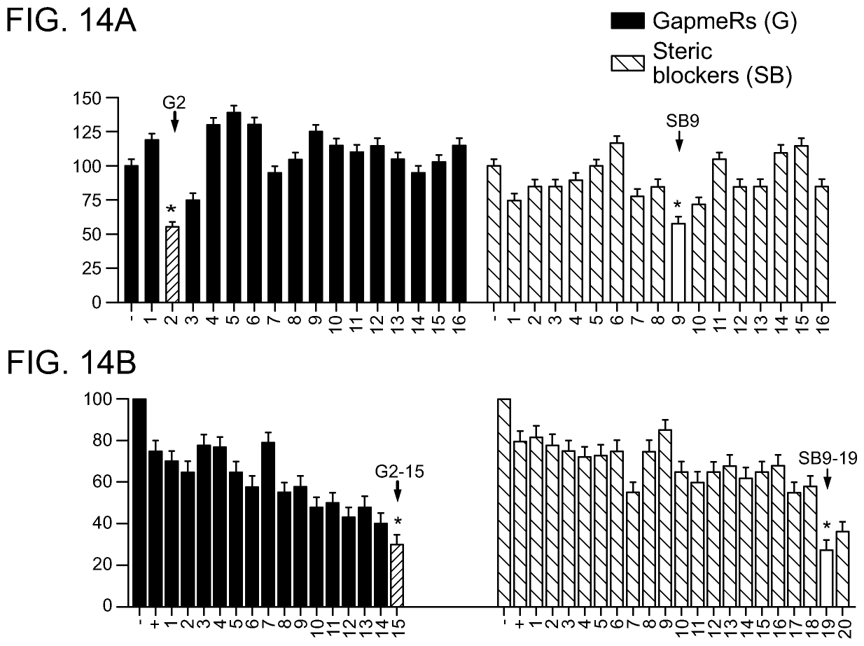

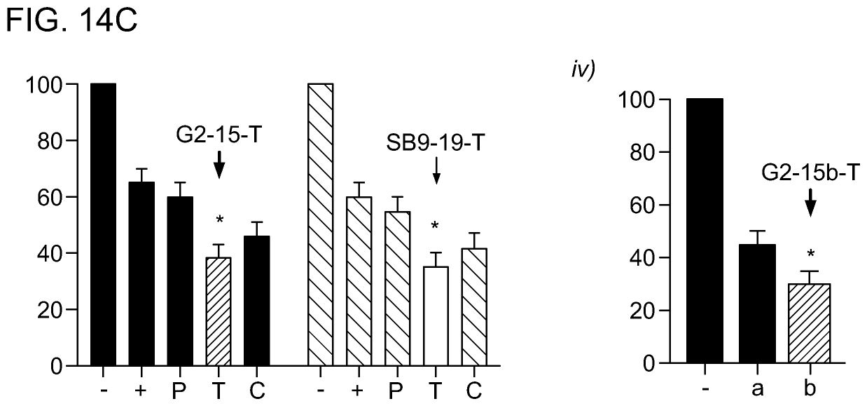

More than 80 fully phosphorothioated (PS), 2′-O-methoxyethyl (2′-MOE)-modified, lipid-conjugated ASOs were screened to explore the therapeutic potential of RROL and to develop inhibitors. These ASOs were screened for their potential to either trigger RNase H-mediated degradation of RROL (gapmeRs) or exert function via an RNase H-independent mechanism (steric blockers) (Puttaraju et al., Nat. Med. 27:526-535 (2021)) (FIG. 7A and FIG. 14A-FIG. 14C). This procedure identified an 18-mer tocopherol (T)-conjugated gapmeR G2-15b-T (“G”) and an 18-mer tocopherol (T)-conjugated steric blocker SB9-19-T (“SB”) as leading compounds, both producing a strong anti-proliferative activity (cell growth inhibition, CGI>50%) in a large panel of MM cell lines as well as CD138+ primary MM cells; while sparing (CGI<50%) non-malignant cell lines (THLE-2, HK-2, HS-5 and 293T) and PBMCs from 3 healthy donors (FIG. 7B). FIG. 7B shows the MM cell lines AMO1, ABZB, ACFZ, H929, MM.IS, MMI.R, U266, LR7, R8226, KMS-11, KMS-12-BM, and CD138+ cells from 2 MM patients (MM-Pt), 4 non-malignant cell lines (THLE-2, HK-2, HS-5 and 293T), and PMBCs from 3 healthy donors. Cell viability was measured 48 h (or 24 h for MM-pt) after transfection with either G2-15b*-TO (G) or SB9-19-TO (SB) or vehicle (−) as control. Cell proliferation is calculated compared to cells transfected with vehicle.

The testing results of the multi-step screen to develop therapeutic ASOs targeting RROL is shown in FIG. 14A-FIG. 14C. The results of step 1 are shown in FIG. 14A, which identified ASO-accessible stretches on RROL, 16 sequences (>20-mer) either in “G” or “SB” configuration for a total of 32 ASOs were tested; based on KD activity in AMO1 cells assessed by qRT-PCR, the sequences G2 (21-mer) and SB9 (22-mer) were selected for further investigation. The results of step 2 are shown in FIG. 14B, which optimized the G2 and SB9 designs, these sequences were fine-tuned to obtain 20-mer (n=8) and 18-mer (n=5) derivative ASOs for G2, and 22-mer (7), 20-mer (n=8) and 18-mer (n=5) derivative ASOs for SB9; based on KD activity in AMO1 cells assessed by qRT-PCR. The 18-mer derivative sequences G2-15 and SB9-19 were selected for further investigation. The results of step 3 are shown in FIG. 14C, which shows the testing of the two selected step 2 molecules conjugated with palmitic acid (P) or cholesterol (C) or tocopherol (T). Based on KD activity in AMO1 cells assessed by qRT-PCR. The TO-conjugated molecules were selected as the leading compounds. The G2-15-T ASO was further optimized by replacing the 10-mer “core” DNA gap with an 8-mer “core” DNA gap (G2-15b-T).

A subcutaneous AMO1 xenograft model in immunocompromised NOD SCID mice was used to assess the in vivo anti-tumor activity of both compounds. Here, a significant reduction of tumor growth was observed 21 days after a treatment cycle with either G2-15b*-TO (G; n=5; tumor growth inhibition, TGI=76%) or SB9-19-TO (SB; n=5; TGI=69%) or vehicle (NK; n=5), as illustrated in FIG. 7C. Analysis of tumors retrieved from mice following this treatment confirmed reduced expression of RROL (FIG. 7D) and its targets (FIG. 7E), target BIM (aka BCL.21.11) (FIG. 7J), as well as reduced levels of tripalmitin (FIG. 7F), a surrogate for the DNL product palmitate (Falchook et al., EClinicalMedicine 34:100797-11 (2021)); demonstrating an efficient uptake of G2-15b-T and SB9-19-T by tumor cells in vivo. Moreover, no overt toxicity was observed in the mice. Data in FIG. 7D and FIG. 7E show samples retrieved from animals treated with G2-15b*-TO (G; n=1) or SB9-19-TO (SB; n=1) or vehicle (NC; n=1) as control. Raw Ct values were normalized to ACTB mRNA and expressed as ΔΔCt values calculated using the comparative cross threshold method. Expression levels in NC were set as an internal reference.

Significant anti-MM activity of G2-15b-T and SB9-19-T was confirmed in an aggressive model of diffused myeloma, in which tumor growth of MOLP8-luc+MM cells is assessed by bioluminescence imaging (BLI) measurement. In this model, tumor growth was significantly antagonized after a treatment cycle with either G2-15b*-TO (G; n=8; TGI=84%) or SB9-19-TO (SB; n=6; TGI=52%) or vehicle (NC; n=11). Treatment with G2-15b*-TO resulted in a tumor clearance in 2 out of 8 mice (25%) (FIG. 7G). On the left of FIG. 7G, a scatter plot showing the analysis of bioluminescence intensity. Black bars indicate median value. Bioluminescence was measured at the end of treatment cycle (day 15). On the right of FIG. 7G, image acquisition is shown. Mice removed from the study due to failed I.V. injection of tumor cells are covered by a black rectangle. Importantly, both inhibitors significantly prolonged the animal survival (FIG. 7H).

Finally, a clinically relevant PDX-NSG mouse model by tail-vein injection of CD138+MM cells obtained from an advanced-stage patient (PDX-NSG) was used. In this model tumor growth was monitored in serum samples using human k light chain as a surrogate. Remarkably, a regression of tumor growth after a treatment cycle with G2-15b-T (G; n=2) was observed (FIG. 7I), and the effects were comparable to bortezomib (BZT; n=2, a positive control) and vehicle control is represented as NC (n=3). Black arrows indicate treatments.

The working example 8 above demonstrates that lncRNA RROL is a leading dependency in MM. RROL host gene, MIR17HG, is often amplified and/or overexpressed in human cancer with driver role. Without intending to be bound by theory of operation, RROL is a regulator of gene expression via chromatin occupancy and interaction with transcription factors and epigenetic modulators, such as MYC and WDR82.

The data also show that the RROL-MYC-WDR82 complex impacts tumor cell metabolism by activating the DNL pathway via the rate-limiting enzyme ACC1. This anabolic pathway is primarily restricted to liver and adipose tissue in normal adults but is reactivated in cancer cells via mechanisms yet to be fully described (Beloribi-Djefaflia et al., Oncogenesis 5: e189-10 (2016); Röhrig and Schulze., Nat Rev Cancer 16:732-749 (2016)). MYC has been implicated in the reprogramming of tumor cell metabolism by activating DNL via ACC1 and other genes (Stine et al., Cancer Discov 5:1024-39 (2015)). In turn, DNL has emerged as an essential pathway for the onset and progression of MYC-driven cancers, that are susceptible to pharmacologic inhibition of ACC1 (Gouw et al., Cell Metab 30:556-572 (2019)). The roles of ACC1 and DNL in tumorigenesis seem particularly relevant in MM, where tumor cells need to adapt their metabolic pathways to meet the high bioenergetic and biosynthetic demand posed by the malignant cell growth coupled with unceasing production of monoclonal immunoglobulin (El Arfani et al., Int J Mol Sci 19:1200-19 (2018); Masarwi et al., JBMR Plus 3: e10173-10 (2019)).

The in vivo working example utilized two of the inventive ASOs that target RROL via different mechanisms of action (i.e., RNase H-dependent or -independent). With the recent advances in RNA medicine (Crooke et al., Cell Metab 27:714-739 (2018); Damase et al., Front Bioeng Biotechnol 9:628137-24 (2021); Sullenger and Nair., Science 352:1417-20 (2016)) the use of ASOs to therapeutically antagonize disease-driver genes is becoming increasing possible (Dhuri et al., J Clin Med 9:2004-24 (2020); Puttaraju et al., Nat Med 27:526-535 (2021)), including in MM therapy (Mondala et al., Cell Stem Cell 28:623-636 (2021); Morelli et al., Blood 132:1050-1063 (2018); (Mondala et al., 2021; Morelli et al., 2018).

In conclusion, this working example establishes RROL as a lncRNA that facilitates MYC-WDR82 protein complex formation and its chromatin binding, impacting lipid metabolism and ultimately tumor cell growth.

All patent publications and non-patent publications are indicative of the level of skill of those skilled in the art to which this disclosure pertains. All these publications (including any specific portions thereof that are referenced) are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. An antisense oligonucleotide (ASO) that binds MIR-17-92a-1 Cluster Host Gene (MIR17HG) pre-RNA under physiological conditions, where the ASO is 15 to about 30 nucleotides in length and the pre-RNA has comprises the nucleic acid sequence of SEQ ID NO: 1.

2. The ASO of claim 1, which binds in the 5′ terminal region of the pre-RNA.

3. The ASO of claim 1, which binds in an intronic region of the pre-RNA.

4. The ASO of claim 1, which binds in an extronic region of the pre-RNA that encodes a portion of Inc-17-92^{TV1}.

5. The ASO of claim 1, which comprises any one nucleic acid sequence of
(SEQ ID NO: 2)
AGTGGCGCGAAGGCGCAGGT,
(SEQ ID NO: 3)
GTGGCGCGAAGGCGCAGGTC,
(SEQ ID NO: 4)
CCTCGCCCGAGGGCGCGAAG,
(SEQ ID NO: 5)
GAGGGCGCGAAGTGGCGCGA,
(SEQ ID NO: 6)
TACTTGCTTGGCTT,
(SEQ ID NO: 7)
CACCGTCCAAATCTAT,
(SEQ ID NO: 8)
AGCACTCAACATCAGC,
(SEQ ID NO: 9)
CACCGTCCAAATCTAT,
(SEQ ID NO: 10)
GTATGACTGGAATAGG,
(SEQ ID NO: 11)
TACAGTGGAAATCGGC,
(SEQ ID NO: 12)
GCGAGCAAACACGAAA,
(SEQ ID NO: 13)
ACTTGGATTGGATGAG.

6-7. (canceled)

8. The ASO of claim 1, wherein a nucleotide thereof is chemically modified.

9. The ASO of claim 8, wherein the nucleotide is modified by a phosphorothioate (PS) internucleoside linkage, a methoxypropylphosphonate (MOP) internucleoside linkage, a 2′-O-methyl (Me) group, a 2′-O-methoxyethylribose (MOE) group, a constrained ethyl (S-cEt) group, a locked nucleic acid (LNA), or a 2′fluoro (2′F) group.

10. The ASO of claim 1, which is 18 nucleotides in length and is conjugated to a lipid optionally palmitic acid, tocopherol, or cholesterol.

11-12. (canceled)

13. The ASO of claim 1, wherein the ASO is single stranded DNA.

14. The ASO of claim 13, wherein the single stranded DNA is flanked on the 3′ terminus and/or the 5′ terminus by one or more modified nucleotides.

15-17. (canceled)

18. The ASO of claim 14, wherein the modified nucleotide comprises one or more 2′-O-methoxyethylribose (MOE) groups.

19. The ASO of claim 18, wherein the single stranded DNA is 15 nucleotides in length or 18 nucelotides in length.

20-21. (canceled)

22. A pharmaceutical composition comprising a therapeutically effective amount of the ASO of claim 1 and a pharmaceutically acceptable carrier.

23. A method of treating a subject in need thereof, the method comprising administering to the subject in need thereof the pharmaceutical composition of claim 22.

24. The method of claim 23, wherein the disease is multiple myeloma, lymphoma, or colorectal cancer.

25. (canceled)

26. The method of claim 23, further comprising administering to the subject a therapeutically effective amount of an acetyl-CoA carboxylase-α (ACC1) inhibitor.

27-29. (canceled)

30. The method of claim 23, further comprising administering to the subject a therapeutically effective amount of an additional active agent selected from bortezomib, melphalan, carfilzomib, dexamethasone, one or more proteasome inhibitors, one or more Immunomodulatory imide drugs (IMiDs), or a combination of two or more thereof.

31-32. (canceled)

33. The method of claim 23, wherein the disease is a liver disease, such as nonalcoholic fatty liver disease (NAFLD), nonalcoholic steatohepatitis (NASH) liver fibrosis, viral hepatitis, or alcoholic liver disease (ALD), or a cancer such as multiple myeloma, a B-cell lymphoma, diffuse large B-cell lymphoma (DLBCL), follicular lymphoma, and Burkitt lymphoma, triple negative breast cancer, pancreatic cancer, liver cancer, or gastric cancer

34. (canceled)

35. The method of claim 23, the method further comprises administering to the subject a therapeutically effective amount of an MYC proto-oncogene, bHLH transcription factor (MYC) inhibitor.

36-40. (canceled)


SEQUENCE LISTING (73 sequences)

SEQ ID NO: 1 - DNA, 6760 bp, Homo sapiens
  source 1..6760: mol_type = genomic DNA
gaagctctcctcgcggggcgggccggccggccgcacccccggcctggggcctccggtcgtagtaaagcgcaggcgggcggggaggcgggagcaggagcccgcggccggccagccgaagatggtggcggctactcctcctggtgagtctgcccgcccctccggcgacggagggaaacctgttgtgtgcggcccgggtctggcgggcggggcggagcggcccggggcggactggcccggggcagcgtggcggcggcggcgtggccggggcgggtctcggccgttggccgccccggcgtgtggcagccgcatctggctgccccctcgctcgcccgcgggccggcggaggggggcagggccggggcgggagggtgggagggggcggcgtgcgcgtggcggccgcgcccgggacccgcgcagaccctgcctgggccgacccgaaggcgggtgggcggacggcgaacacaatggcccctcggggagaggacgtgcgaggcccgtgccttctccggggcccggggcgcgcgcggggcgtggggtctctgggtaggaaagtttctcccgagggcgagagttaaagcgcctccagaacaaagcggcggcggcggcggcacatggggcaggccgcgggccgggagggggcgcgcccacgaggtacctgcgcgccagcgggcggcgtggcgtgggcgggagcccgcggttccccaaactttgtacgcgcgagggtgggcggaggggcgccgagatcggcgcggcctgggcgccacccccgctccgcgtgggctttgttagcccgcgtgggcagcctcggggcggggcccgcaacttccccgccgtggccctcggaggaggccgcagtcggcctcagccgcggcgtggagccgcctgcgcccggccgcttgctgggagtgtggcgcgggagggccagcccggctcggcgggagcggcgtccccgccgccatgttcctgcggggcgggctgcacgggggtgagggcgggggacatggcggcgactgcgcgccgccgccgattgttcccggcttaggcctcgggccgcgtgcgacgggcaccgcggccgcggagcccccgcccctctgggccgggctcgggggggctgggggacacaaaggaggggcggcgcgcccgcgtccccgccgcactcgggcctcggcgccgccggtcgccgcgcggctgccgccgggaaacgggttgggggggttgccgcgtccggcggggcctgactctgacccgccgccccctggcggctacgcggagaatcgcagggccgcgctcccccttgtgcgacatgtgctgccggcccgggctccatgagcgtggcgggcactttgcagtctcgggtgttcctgcccggtcttctgttcctaaactgcagcaaagggaaaaggaactgaaaaaggcaggctcgtcgttgcaatatcaccaaaagagaaaattaacggcatgccatcaggaccacagcagttggagaaacaactctttatcccggcttgcagccacgaggtcttgattgggggaggggtggtgaagaatagtctgtgggctgctttttttttttccttttactggagctgtacagtggagtcggtgattgctgctgatcataatcaagtattttaggagcttatttagacatgtatctgatagctaaggatttttcaactttattctcttacgtatttttcaactgtaaattattgggcttttaaatcctgctagtattgctcgactcttactctcacaaatggatggaattaattgctgttaggaggttggaaaatagcaaatatagatttggacggtggtagtaattttgagcaaataatgttttatcttttttttccttatttttccctattccagtcatacacgtggacctaactgcaccagtagcttttctgagaatacttgctgaaaaggaagttttctggaatgggtaagtgtattctgattttcttgaacttttcttaaaaacaaatttttcttgctattaaagttgaataaataggattggtttcttagagagtaaaagtaggtgtttctttctttagacaatgtaccttttctgaaaaactaactcattaagtacggatttgctaattttaaggtagtaaaattacagtgtaaatattcctgtacatttttggaaactggcttatgcagtttacgaaatataattttagaccctcttttaagttgggtgataaagtagatataacctgagatgatagatttaaacaggatatttacgttctgctacaattgactgataacacttgaagtgtagtctgaacagtaattttgttaatcatttcaacaagtatttgctaagtggaagccagaagaggaggaaaatgttttgccacgtggatgtgaagatttcctctaaaaggtacacatggactaaattgcctttaaatgttccaaaattagttctcatttatttgcagtctcattttgttttgttttttttctctatgtgtcaatccatttgggagaggccagccattggaagagccaccacttccagtgctagttggatggttggttatgattgccttctgtaaagaattcttaaggcataaatacgtgtctaaatggacctcatatctttgagataattaaactaattttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcaaagtgcttacagtgcaggtagtgatatgtgcatctactgcagtgaaggcacttgtagcattatggtgacagctgcctcgggaagccaagttgggctttaaagtgcagggcctgctgatgttgagtgctttttgttctaaggtgcatctagtgcagatagtgaagtagattagcatctactgccctaagtgctccttctggcataagaagttatgtattcatccaataattcaagccaagcaagtatataggtgttttaatagtttttgtttgcagtcctctgttagttttgcatagttgcactacaagaagaatgtagttgtgcaaatctatgcaaaactgatggtggcctgctatttccttcaaatgaatgatttttactaattttgtgtacttttattgtgtcgatgtagaatctgcctggtctatctgatgtgacagcttctgtagcactaaagtgcttatagtgcaggtagtgtttagttatctactgcattatgagcacttaaagtactgctagctgtagaactccagcttcggcctgtcgcccaatcaaactgtcctgttactgaacactgttctatggttagttttgcaggtttgcatccagctgtgtgatattctgctgtgcaaatccatgcaaaactgactgtggtagtgaaaagtctgtagaaaagtaagggaaactcaaacccctttctacacaggttgggatcggttgcaatgctgtgtttctgtatggtattgcacttgtcccggcctgttgagtttggtggggattgtgaccagaagattttgaaaattaaatattactgaagatttcgacttccactgttaaatgtacaagatacatgaaatattaaagaaaatgtgtaactttttgtgtaaatacatcttgtcttgttttcattcaaaaacatttcacttttggggttgcgtgtcagatttggcagtataaattctggctatattttttgttgttagatttatttggctgttaagtattgcgatatgactaaacatactgtatacctgatgatcatctgtaaagttagagtatatctttttgctttctttggagttagtgttattccaggatattttacttaatctaaaagttaatttatgttgctcatatattactcaagtatttaaatttagagagaatgccgctctgtttaaagcaatgtgtaaagatgagtttttttaagcatggaatttagggttggggtacaatttgtttctattaagcaagtaccagtttaccaatacatgagtaactgaagtgtaactgttaaatgcttgtatactagtttttctttctgattgtcagtgatttataagctataaatgaccaaggtcctcagactgcttttagcatctgcaacttaaaaaaatgggagttagaaaaagaacaaatgctaaatagagtaacagttaaatgtatgtgtacactcttcccaaatgccaagagtgcagcggtggggtgagattcagatattcatttatttctaagtctgtagttaacatttatgttccctactccctacgtaagccagactttggcaacagtgatagttgattccaggcttatttgacttaaagtcactgaagtggaaactaagaagtggcagttagtgttttacccagcatttctgccttctctcttttcttcatgtgtttttgtctctagcctatgtgtatttgtgtagaataatgtgggatacctgaataatagatttaaaaggaccaagtggtaaaattgggcccaagctgaagtacaggcaaacttgatgtttgaaagataagttttgagaaatgtcattgtattttggagtaaaagaggctatcttagtaataagaaataaacttccataacactaggttagaccacccaataaatctagaaatcagcttttaaaaatattgtctgaagtctaacaaaagttttcacctctaatgtgttctttaagaaatttaaggaacttagccttggattcctgaatagaaaggtaagaattctatcattctggagttgatgaaaacataaattttcaggatgtgaaatgaacagtgatttataaaatggaaatcaaattgtacattagcagagttcttaagctttttgaattgaaggagacctaataattgtgtctttttggttatttagtgacaaacgtggctttcaaactatgcttaaaaagttccggctggacacggtggctcacacctataatcctagcacttggggaggctgaggcagacggattacctgaggtcaggagttcgagaccaacctggccgacatggtgaaacgctgtctctactaaaaatataaaaaattagccgggtgcagtggcgtgcacctgtaatcccagctactctggaggctgaggcaggagaatcacctgaacctgggaggtggaggtttcagtgagctgagatcctgccactgcactccagcctgggcgcaagaccaagacttaaacgcaaaaaaaaaaaaaaaaaaaaaaaaaaagtttcataatacagcatggtctggtagtttgcaaaatggtgtgcttttggggagatacactagcaatttttttaaaaactggaacagtgtgataggaagcctgctggatgatttcttaaatattctaaaatgtaagtcaaatatgttttaataacaaagacttaaatggcttttctccctagagactgaaactagtattcattgtgttcagaacttaattgggcttgaactgagatttaaatctaataaacaagttaataaatgtgtatgttttgttgtgggtttggtagtgatctgtggttctatagggtttaataggaattgcttttgatttgtttctggctttagaatgtgaggcaaattttacattcttggttctattaagattttcttaggcatgctaacatgccaacaaaaagccatgtaagtattgtataaaaagattcacattgttaatttagccattttgaaattcagatgagtgagcaagttgataatggcctcatctctgacctgagaaaaaacaactttgacccttgttcttaaaatgctttaaccttgaagttgcttgagacttaagaggtcatgttgctttaggtttaataaatagccttaactatttggaggggaaaaaatgggtcaacttttttttttttttttggcgtttgcatgtacaactttctatttttagcctatatttggaaagaaagcacttaacattttaggaattctttttaaagctgcttgcaaagtgttggtgattttactgaaaacttttgagatcttcattttacaggcagacctgtctaactacaagccagacttgggttttctcctgtagtttgaagacacactgactcctgacaaaatgcagcctgcaacttcctggagaacaactcagtgtcacattaaagtttattatgtatttaatgatacactgtttaattgacagttttgcatagtttgtctaactttagagaattaagagcctctcaactgagcagtaaaggtaaggagagctcaatctgcacagagccagtttttagtgtttgatggaaataagatcatcatgcccacttgagacttcagattattctttagcttagtggttgtatgagttacatcttattaaagtcgaaattaatgtagttttctgccttgataacatttcatatgtggtattagttttaaagggtcattaggaaaatgcacatattccatgaattttaagacccatagaaaagttgaagaatgcttaattttcttatccagtaatgtaaacacagagacagaacattgagatgtgcctagttctgtatttacagtttggtctggctgtttgagttctagcgcatttaatgttaataaataaaatactgcattttaaagctgttaagaaattgtccagaacgagaatattgaaataaaaacttcaaggttatt

SEQ ID NO: 2 - DNA, 20 bp, synthetic construct
  source 1..20: mol_type = other DNA
agtggcgcgaaggcgcaggt

SEQ ID NO: 3 - DNA, 20 bp, synthetic construct
  source 1..20: mol_type = other DNA
gtggcgcgaaggcgcaggtc

SEQ ID NO: 4 - DNA, 20 bp, synthetic construct
  source 1..20: mol_type = other DNA
cctcgcccgagggcgcgaag

SEQ ID NO: 5 - DNA, 20 bp, synthetic construct
  source 1..20: mol_type = other DNA
gagggcgcgaagtggcgcga

SEQ ID NO: 6 - DNA, 14 bp, synthetic construct
  source 1..14: mol_type = other DNA
tacttgcttggctt

SEQ ID NO: 7 - DNA, 16 bp, synthetic construct
  source 1..16: mol_type = other DNA
caccgtccaaatctat

SEQ ID NO: 8 - DNA, 16 bp, synthetic construct
  source 1..16: mol_type = other DNA
agcactcaacatcagc

SEQ ID NO: 9 - DNA, 16 bp, synthetic construct
  source 1..16: mol_type = other DNA
caccgtccaaatctat

SEQ ID NO: 10 - DNA, 16 bp, synthetic construct
  source 1..16: mol_type = other DNA
gtatgactggaatagg

SEQ ID NO: 11 - DNA, 16 bp, synthetic construct
  source 1..16: mol_type = other DNA
tacagtggaaatcggc

SEQ ID NO: 12 - DNA, 16 bp, synthetic construct
  source 1..16: mol_type = other DNA
gcgagcaaacacgaaa

SEQ ID NO: 13 - DNA, 16 bp, synthetic construct
  source 1..16: mol_type = other DNA
acttggattggatgag

SEQ ID NO: 14 - DNA, 16 bp, synthetic construct
  source 1..16: mol_type = other DNA
gctcccttcaatccaa

SEQ ID NO: 15 - DNA, 22 bp, synthetic construct
  source 1..22: mol_type = other DNA
cccggccgcctccccccgtccc

SEQ ID NO: 16 - DNA, 20 bp, synthetic construct
  source 1..20: mol_type = other DNA
ccctcccaccctcccccgcc

SEQ ID NO: 17 - DNA, 21 bp, synthetic construct
  source 1..21: mol_type = other DNA
cccccccgaccccctgtgttt

SEQ ID NO: 18 - DNA, 21 bp, synthetic construct
  source 1..21: mol_type = other DNA
tcctccaaccttttatcgttt

SEQ ID NO: 19 - DNA, 19 bp, synthetic construct
  source 1..19: mol_type = other DNA
tcgtccttttttctcttgt

SEQ ID NO: 20 - DNA, 20 bp, synthetic construct
  source 1..20: mol_type = other DNA
ctccccttttttacccagtt

SEQ ID NO: 21 - DNA, 21 bp, synthetic construct
  source 1..21: mol_type = other DNA
ccctctccggtcggtaacctt

SEQ ID NO: 22 - DNA, 22 bp, synthetic construct
  source 1..22: mol_type = other DNA
tcgtccttttttctcttgtagt

SEQ ID NO: 23 - DNA, 22 bp, synthetic construct
  source 1..22: mol_type = other DNA
ccacccctaacactggtcttct

SEQ ID NO: 24 - DNA, 19 bp, synthetic construct
  source 1..19: mol_type = other DNA
cccctccgccctcgtcctc

SEQ ID NO: 25 - DNA, 18 bp, synthetic construct
  source 1..18: mol_type = other DNA
tctctcattttcatccac

SEQ ID NO: 26 - DNA, 19 bp, synthetic construct
  source 1..19: mol_type = other DNA
agagtaaaacaaaacaaaa

SEQ ID NO: 27 - DNA, 20 bp, synthetic construct
  source 1..20: mol_type = other DNA
acaaaatagaaaaaaaagga

SEQ ID NO: 28 - DNA, 20 bp, synthetic construct
  source 1..20: mol_type = other DNA
ataaaaagggataaggtcag

SEQ ID NO: 29 - DNA, 20 bp, synthetic construct
  source 1..20: mol_type = other DNA
tcgtccttttttctcttgta

SEQ ID NO: 30 - DNA, 19 bp, synthetic construct
  source 1..19: mol_type = other DNA
ccctatggacttattatct

SEQ ID NO: 31 - DNA, 20 bp, synthetic construct
  source 1..20: mol_type = other DNA
gccctcccaccctcccccgc

SEQ ID NO: 32 - DNA, 20 bp, synthetic construct
  source 1..20: mol_type = other DNA
cgccctcccaccctcccccg

SEQ ID NO: 33 - DNA, 20 bp, synthetic construct
  source 1..20: mol_type = other DNA
ccgccctcccaccctccccc

SEQ ID NO: 34 - DNA, 20 bp, synthetic construct
  source 1..20: mol_type = other DNA
cccgccctcccaccctcccc

SEQ ID NO: 35 - DNA, 20 bp, synthetic construct
  source 1..20: mol_type = other DNA
cctcccaccctcccccgccg

SEQ ID NO: 36 - DNA, 20 bp, synthetic construct
  source 1..20: mol_type = other DNA
ctcccaccctcccccgccgc

SEQ ID NO: 37 - DNA, 20 bp, synthetic construct
  source 1..20: mol_type = other DNA
tcccaccctcccccgccgca

SEQ ID NO: 38 - DNA, 20 bp, synthetic construct
  source 1..20: mol_type = other DNA
cccaccctcccccgccgcac

SEQ ID NO: 39 - DNA, 18 bp, synthetic construct
  source 1..18: mol_type = other DNA
ccctcccaccctcccccg

SEQ ID NO: 40 - DNA, 18 bp, synthetic construct
  source 1..18: mol_type = other DNA
cctcccaccctcccccgc

SEQ ID NO: 41 - DNA, 18 bp, synthetic construct
  source 1..18: mol_type = other DNA
ctcccaccctcccccgcc

SEQ ID NO: 42 - DNA, 18 bp, synthetic construct
  source 1..18: mol_type = other DNA
gccctcccaccctccccc

SEQ ID NO: 43 - DNA, 18 bp, synthetic construct
  source 1..18: mol_type = other DNA
cgccctcccaccctcccc

SEQ ID NO: 44 - DNA, 18 bp, synthetic construct
  source 1..18: mol_type = other DNA
tcccaccctcccccgccg

SEQ ID NO: 45 - DNA, 18 bp, synthetic construct
  source 1..18: mol_type = other DNA
cccaccctcccccgccgc

SEQ ID NO: 46 - DNA, 22 bp, synthetic construct
  source 1..22: mol_type = other DNA
accacccctaacactggtcttc

SEQ ID NO: 47 - DNA, 22 bp, synthetic construct
  source 1..22: mol_type = other DNA
aaccacccctaacactggtctt

SEQ ID NO: 48 - DNA, 22 bp, synthetic construct
  source 1..22: mol_type = other DNA
aaaccacccctaacactggtct

SEQ ID NO: 49 - DNA, 22 bp, synthetic construct
  source 1..22: mol_type = other DNA
caaaccacccctaacactggtc

SEQ ID NO: 50 - DNA, 21 bp, synthetic construct
  source 1..21: mol_type = other DNA
cacccctaacactggtcttca

SEQ ID NO: 51 - DNA, 21 bp, synthetic construct
  source 1..21: mol_type = other DNA
acccctaacactggtcttcaa

SEQ ID NO: 52 - DNA, 20 bp, synthetic construct
  source 1..20: mol_type = other DNA
ccctaacactggtcttcaaa

SEQ ID NO: 53 - DNA, 20 bp, synthetic construct
  source 1..20: mol_type = other DNA
cctaacactggtcttcaaaa

SEQ ID NO: 54 - DNA, 20 bp, synthetic construct
  source 1..20: mol_type = other DNA
ccacccctaacactggtctt

SEQ ID NO: 55 - DNA, 20 bp, synthetic construct
  source 1..20: mol_type = other DNA
cacccctaacactggtcttc

SEQ ID NO: 56 - DNA, 20 bp, synthetic construct
  source 1..20: mol_type = other DNA
acccctaacactggtcttct

SEQ ID NO: 57 - DNA, 20 bp, synthetic construct
  source 1..20: mol_type = other DNA
accacccctaacactggtct

SEQ ID NO: 58 - DNA, 20 bp, synthetic construct
  source 1..20: mol_type = other DNA
aaccacccctaacactggtc

SEQ ID NO: 59 - DNA, 19 bp, synthetic construct
  source 1..19: mol_type = other DNA
cccctaacactggtcttca

SEQ ID NO: 60 - DNA, 19 bp, synthetic construct
  source 1..19: mol_type = other DNA
ccctaacactggtcttcaa

SEQ ID NO: 61 - DNA, 18 bp, synthetic construct
  source 1..18: mol_type = other DNA
acccctaacactggtctt

SEQ ID NO: 62 - DNA, 18 bp, synthetic construct
  source 1..18: mol_type = other DNA
cacccctaacactggtct

SEQ ID NO: 63 - DNA, 18 bp, synthetic construct
  source 1..18: mol_type = other DNA
ccacccctaacactggtc

SEQ ID NO: 64 - DNA, 18 bp, synthetic construct
  source 1..18: mol_type = other DNA
cccctaacactggtcttc

SEQ ID NO: 65 - DNA, 18 bp, synthetic construct
  source 1..18: mol_type = other DNA
ccctaacactggtcttct

SEQ ID NO: 66 - DNA, 20 bp, synthetic construct
  source 1..20: mol_type = other DNA
ccggcgagatctccgtgctc

SEQ ID NO: 67 - DNA, 20 bp, synthetic construct
  source 1..20: mol_type = other DNA
gatgtggtcattcgtcatga

SEQ ID NO: 68 - DNA, 20 bp, synthetic construct
  source 1..20: mol_type = other DNA
cctgcaacttcctggagaac

SEQ ID NO: 69 - DNA, 20 bp, synthetic construct
  source 1..20: mol_type = other DNA
gtctcaagtgggcatgatga

SEQ ID NO: 70 - DNA, 21 bp, synthetic construct
  source 1..21: mol_type = other DNA
gaccctcttttaagttgggtg

SEQ ID NO: 71 - DNA, 20 bp, synthetic construct
  source 1..20: mol_type = other DNA
tggcaaaacattttcctcct

SEQ ID NO: 72 - DNA, 26 bp, synthetic construct
  source 1..26: mol_type = other DNA
tttctctcttgcagagtgaggtgtgg

SEQ ID NO: 73 - DNA, 24 bp, synthetic construct
  source 1..24: mol_type = other DNA
tacaaaggcacggagagagcaagt